(19) United States
(12) Patent Application Publication
(10) Pub. No.: US 2011/0262430 A1
(43) Pub. Date: Oct. 27, 2011

(54) NOVEL ANTIBODIES

(76) Inventors: Jonathan Henry Ellis, Hertfordshire (GB); Paul Andrew Hamblin, Hertfordshire (GB)

(21) Appl. No.: 12/671,607

(22) PCT Filed: Jul. 28, 2008
(86) PCT No.: PCT/EP2008/059900

§ 371 Date: Jul. 8, 2011

Related U.S. Application Data
(60) Provisional application No. 60/953,210, filed on Aug. 1, 2007.

(51) Int. Cl. A61K 39/395; A61P 29/00; C12P 21/00; A61P 35/00; C07K 16/28; C07K 16/40

(52) U.S. Cl. 424/133.1; 530/387.3; 435/69.6

(57) ABSTRACT

The present invention relates to antibodies or antigen binding fragments thereof which specifically binds to IGF-IR, specifically hIGF-1R. Also disclosed are antibody preparations comprising antibodies or antigen binding fragments of the invention. Methods of producing such antibodies or antigen binding fragments and uses thereof are also included within the scope of the present invention.

The present invention relates to antibodies and antigen binding fragments thereof that specifically bind human Insulin-like Growth Factor Receptor (hIGF-1R). The present invention also concerns methods of treating diseases or disorders with said antibodies and antigen binding fragments thereof, pharmaceutical compositions comprising said antibodies and antigen binding fragments thereof and methods of manufacture.

### BACKGROUND

The human insulin-like growth factor receptor (also known as IGF-1R, CD221 or EC 2.7.112) is a tyrosine kinase receptor with 70% homology to the insulin receptor. The receptor is activated by two ligands—IGF-I and IGF-II which bind the receptor with high affinity. The receptor is a disulphide linked aβ dimer, denoted (aβ)₂. The α-chain is entirely extracellular whilst the β-chain is membrane-spanning and has both an extracellular domain and an intracellular signalling domain. Ligand-mediated receptor activation triggers intracellular events including activation of MAPK and PI3K-protein kinase B pathways. Whilst IGF-1R is known to have an essential role in normal foetal and postnatal growth and development, it has assumed an important role in cancer biology and has been implicated in a number of biological pathways such as mitogenesis, transformation and protection from apoptosis (reviewed extensively in Baserga et al. (1997, 2003), Hasnain et al. (2000), Larsson et al. (2005), Romano (2003)). Furthermore the levels of receptor expression are known to be up-regulated on a variety of tumours types (reviewed by Khandwala et al. (2000)) and increased levels of the ligand IGF-I are associated with an increased risk of developing prostate cancer (Chan et al. (1998)). Antagonists of the IGF-1R signalling pathway are known for their anti-tumour effects in vitro and in vivo (reviewed in Hofmann et al. (2005) and Zhang et al. (2004)). Approaches include neutralising antibodies (see Kull et al. (1983) and Li et al, (1993), Xiong et al. (1992), Burtrum et al. (2003), Cohen et al. (2005), Maloney et al. (2003), Jackson-Booth et al. (2003)), anti-sense (see Resnicoff et al. (1994), Lee et al. (1996), Muller et al. (1998), Trojan et al. (1993), Lie et al. (1998), Shapiro et al. (1994)), dominant negative mutants (Prager et al. (1994)) and small molecule tyrosine kinase inhibitors (see Hopfner et al. (2006) and IGF-binding proteins (IGFBPs—see Nickerson et al. (1997)) Known monoclonal antibodies include those described in: WO99/60023, WO03/100008, WO02/053596, WO04/071529, EP0629240B, WO03/059951, WO03/106621, WO04/083248, WO04/087756, US2006452167A.

Antibody structures are well known in the art and in particular it is known that the heavy chain constant region has a glycosylated sugar chain, this may be an N-glycoside linked sugar chain for example N-acetylyglucosamine and it may or may not be fucosylated.

Methods for measuring levels of fucosylation are well known in the art for example, for a population of antibodies, acid hydrolysis can be used to remove the monosacchharides of the glycosylated sugar chain from the antibody and these can be labelled with a dye such as 2-aminobenzoic acid (2-AA). Reverse phase high performance liquid chromatography with fluorescence detection can then be carried out and a standard curve constructed for sample quantitation. The ratio of fucose to mannose per antibody population can then be calculated

Thus there is a need for antibodies with improved effector function, for example with improved ADCC and/or CDC function.

### BRIEF DESCRIPTION OF THE FIGURES

FIG. 1: Binding of purified murine monoclonal antibodies to human IGF-1R as determined by ELISA.

FIG. 2A-E: Binding of purified 6E11 chimeric and 6E11 humanised antibodies to human IGF-1R as determined by ELISA. In FIG. 2A, the binding curve for H0L0 was shifted to the right due to the fact that the antibody was at very low concentration and could not be accurately quantified. In FIG. 2D, whist the overall trend was similar, the signal was reduced compared to other assays.

FIG. 3: Down-regulation of IGF-1R receptor following incubation of 3T3/LISN c4 cells for 24 hours with purified murine monoclonal antibodies to human IGF-1R

FIG. 4: Inhibition of receptor phosphorylation mediated by purified murine monoclonal antibodies 6E11, 5G4 and 15D9,

FIG. 5: shows an example of the inhibition of receptor phosphorylation mediated by H0L0 and H0L0 IgG1m(AA) and H1L0 and H10L0 IgG1m(AA) in comparison to 6E11c.

FIG. 6: shows an example of the inhibition of receptor phosphorylation mediated by H0L0 and H0L0 IgG1m(AA) and H1L0 and H10L0 IgG1m(AA).

FIG. 7A: shows an example of the activity of various purified murine monoclonal antibodies in the competition ELISA.

FIG. 7B: shows an example of the activity of H1L0 in the competition ELISA in comparison to 6E11c.

FIG. 8A-C: Competition ELISA to demonstrate the ability of purified 6E11 murine monoclonal or 6E11 chimeric or 6E11 humanised antibodies to inhibit the binding of IGF-1R receptor to a second neutralising antibody. In FIG. 8A, H0L0 and H0L0 IgG1m(AA) showed an increased signal compared to the repeat assays shown in FIGS. 8B and 8C.

FIG. 9A: Binding of purified murine monoclonal antibodies to recombinant cynomolgus macaque IGF-1R as determined by ELISA.

FIG. 9B: Binding of purified humanised monoclonal antibodies to recombinant cynomolgus macaque IGF-1R in comparison to the 6E11 chimera (6E11c).

FIG. 10: Insulin receptor binding ELISA using purified murine monoclonal antibodies. In contrast to the positive control antibody (R&D Systems MAB15441), purified antibodies 6E11, 5G4 and 15D9 showed no binding to the insulin receptor at concentrations up to 10 μg/ml.

FIG. 11: FACS assay to demonstrate that the antibodies recognize the Colo205 tumour cell line expressed human IGF-1R

FIG. 12: Immunohistochemistry on frozen tissue samples of tumour and normal prostate samples using purified murine monoclonal antibody

FIG. 13: Immunohistochemistry on frozen tissue samples of tumour breast samples using purified murine monoclonal antibody

FIG. 14: Immunohistochemistry on frozen tissue samples of tumour breast samples using purified H1L0 humanised and 6E11 chimeric monoclonal antibodies.

FIG. 15 Inhibition of IGF-I mediated proliferation of 3T3/LISN c4 cells inhibited by purified murine monoclonal antibodies

FIG. 16: Inhibition of IGF-I mediated proliferation of 3T3/LISN c4 cells inhibited by purified H1L0 humanised or 6E11 chimeric monoclonal antibodies

FIG. 17A-E: Inhibition of IGF-I mediated proliferation of 3T3/LISN c4 cells inhibited by purified humanised or purified murine 6E11 monoclonal antibodies

FIG. 18: Inhibition of IGF-I mediated cell cycling by purified murine monoclonal antibodies as determined by propidium iodide staining and flow cytometry.

FIG. 19: shows that the presence of IGF-I affords NCI-H838 cells some protection from camptothecin induced apoptosis. The addition of 6E11 reversed the IGF-1 mediated protection from apoptosis

FIG. 20: Absence of agonistic activity of purified murine monoclonal antibody in the presence of cross-linking antibody. 3T3/LISN c4 cells were incubated with the antibody samples in the absence of ligand and in the presence of anti-mouse cross-linking antibody. Receptor phosphorylation levels were assessed by ELISA.

FIG. 21: Inhibition of 3T3/LISN c4 tumour growth in nude mice following treatment with 6E11 monoclonal antibody

FIG. 22: Inhibition of 3T3/LISN c4 tumour growth in nude mice following treatment with 6E11 monoclonal antibody

FIG. 23: Inhibition of Colo205 tumour growth in nude mice following treatment with 6E11 monoclonal antibody

FIG. 24: Oligosaccharide composition of A) Antibody IGF1R-E and B) Antibody IGF1R-F

FIG. 25: Binding ELISA to recombinant human IGF-1R

FIG. 26: ADCC assay with anti-CD20 antibodies FIG. 27: Kinetics of binding of anti-CD20 antibodies to FcγRIIIa—A) FcγRIIIa (Phe variant), B) FcγRIIIa (Val variant)

FIG. 28: Kinetics of binding of anti-IGF-1R antibodies to FcγRIIIa—A) FcγRIIIa (Phe variant), B) FcγRIIIa (Val variant)

### SUMMARY OF INVENTION

In one embodiment the invention provides an antibody preparation comprising antibodies which comprise an immunoglobulin heavy chain constant region, or antigen binding fragments thereof which are linked to an immunoglobulin heavy chain constant region wherein said immunoglobulin heavy chain constant region confers an effector function to the antibody or antigen binding fragment, and wherein said antibody or antigen binding fragment specifically binds to a growth factor receptor and wherein said immunoglobulin heavy chain constant region is mutated in at least 2 positions and has an altered glycosylation profile such that the ratio of fucose to mannose is 0.8:3 or less so that said antibody or antigen binding fragment has an enhanced effector function in comparison with an equivalent antibody or antigen-binding fragment with an immunoglobulin heavy chain constant region lacking said mutations and altered glycosylation profile.

Also provided is a method of producing an antibody as described herein comprising expressing in a cell line an antibody or antigen binding fragment thereof which has been adapted to regulate the presence or absence of binding of fucose to an N-glycoside linked sugar chain which binds to the immunologically functional molecule.

In another embodiment is provided a kit-of-parts comprising the composition described herein together with instructions for use.

Also provided is a method of treating a human patient afflicted with cancer which method comprises the step of administering a therapeutically effective amount of the antibody preparation described herein.

### DETAILED DESCRIPTION OF INVENTION

The present invention provides an antibody or antigen binding fragment thereof which specifically binds IGF-1R, for example which specifically binds hIGF-1R.

In one embodiment of the present invention there is provided an antibody or antigen binding fragment thereof which specifically binds hIGF-1R and neutralises the activity of hIGF-1R, which comprises a heavy chain variable domain which specifically binds IGF-1R comprising CDR H3 of SEQ. ID. NO: 1 or variants thereof in which one or two amino acid residues within CDR H3 differ from the amino acid residues in the corresponding position in SEQ. ID. NO: 1.

In one embodiment of the present invention these differences in amino acid residues are conservative substitutions.

In another embodiment of the invention there is provided an antibody or antigen binding fragment thereof which specifically binds IGF-1R and comprises a CDRH3 which is a variant of the sequence set forth in SEQ. I.D. NO:1 in which one or two residues within said CDRH3 of said variant differs from the residue in the corresponding position in SEQ. I.D. NO:1 in position 7 and/or position 9 (where the first residue is position 1, W, and where the last residue, V, is in position 14).

In a further embodiment of the invention there is provided an antibody or antigen binding fragment thereof which specifically binds IGF-1R and comprises a CDRH3 which is a variant of the sequence set forth in SEQ. I.D. NO:1 in which one or two residues within said CDRH3 of said variant differs from the residue in the corresponding position in SEQ. I.D. NO:1 by a substitution of R to S at position 7, or by a substitution of K to R at position 9, or by a substitution of R to S at position 7 and K to R at position 9.

In another embodiment of the invention there is provided an antibody or antigen binding fragment thereof further comprising one or more of the following sequences CDRH2 as set out in SEQ. ID. NO: 2, CDRH1 as set out in SEQ. ID. NO: 3, CDRL1 as set out in SEQ. ID. NO: 4, CDRL2 as set out in SEQ. ID. NO: 5, and CDRL3 as set out in SEQ. ID. NO: 6.

In one embodiment of the present invention one or more of the CDR's of the antibody or antigen binding fragment thereof may comprise variants of the CDR's set out in the sequences listed above. Each variant CDR will comprise one or two amino acid residues which differ from the amino acid residue in the corresponding position in the sequence listed. Such substitutions in amino acid residues may be conservative substitutions, for example, substituting one hydrophobic amino acid for an alternative hydrophobic amino acid, for example substituting Leucine with Valine, or Isoleucine.

In a further embodiment of the invention there is provided an antibody or antigen binding fragment thereof comprising CDRH3 and further comprises one or more of the following sequences CDRH2: SEQ. ID. NO: 2, CDRH1: SEQ. ID. NO: 3, CDRL1: SEQ. ID. NO: 4, CDRL2: SEQ. ID. NO: 7, and CDRL3: SEQ. ID. NO: 6.

In yet a further embodiment of the invention there is provided an antibody or antigen binding fragment thereof comprising CDRH3 and further comprises one or more of the following sequences CDRH2: SEQ. ID. NO: 2, CDRH1: SEQ. ID. NO: 3, CDRL1: SEQ. ID. NO: 4, CDRL2: SEQ. ID. NO: 7, and CDRL3: SEQ. ID. NO: 6 wherein one or more of the CDR's may be replaced by a variant thereof, each variant CDR containing 1 or 2 amino acid substitutions.

In one embodiment the antibody or antigen binding fragment thereof of the present invention comprises CDR H3 of SEQ. ID. NO: 1 and CDR H1 of SEQ. ID. NO: 3. In a further embodiment the antibody or antigen binding fragment thereof comprises CDRH3 of SEQ ID NO: 1 and CDR L2 of SEQ. ID. NO: 7. In yet a further embodiment the antibody or antigen binding fragment thereof of the present invention comprises CDR H3 of SEQ. ID. NO: 1 and CDR H1 of SEQ. ID. NO: 3. and CDR L2 of SEQ. ID. NO: 7.

In another embodiment of the present invention there is provided an antibody or antigen binding fragment thereof according to the invention described herein and further comprising the following CDR's:

### CDRH1: SEQ. ID. NO: 3

### CDRH2: SEQ. ID. NO: 2

### CDRH3: SEQ. ID. NO: 1

### CDRL1: SEQ. ID. NO: 4

### CDRL2: SEQ. ID. NO: 7

### CDRL3: SEQ. ID. NO: 6

In another embodiment of the invention there is provided an antibody or antigen binding fragment thereof which specifically binds IGF-1R and comprises CDR's which are variants of the sequences set forth above.

In another embodiment of the present invention there is provided an antibody or antigen binding fragment thereof which specifically binds IGF-1R and comprises a heavy chain variable domain of SEQ. ID. NO: 8 and a light chain variable domain of SEQ. ID. NO: 9, or a heavy chain variable domain of SEQ. ID. NO: 10 and a light chain variable domain of SEQ. ID. NO: 11, or a heavy chain variable domain of SEQ. ID. NO: 12 and a light chain variable domain of SEQ. ID. NO: 13, or a heavy chain variable domain of SEQ. ID. NO: 14 and a light chain variable domain of SEQ. ID. NO: 16, or a heavy chain variable domain of SEQ. ID. NO: 15 and a light chain variable domain of SEQ. ID. NO: 16.

In another embodiment of the invention there is provided an isolated heavy chain variable domain of an antibody comprising SEQ. I.D. NO: 12, SEQ. I.D. NO: 14 or SEQ. I.D. NO: 15, for example it comprises SEQ. I.D. NO: 12.

In another embodiment of the present invention there is provided an antibody or antigen binding fragment thereof comprising CDR's according to the invention described herein, or heavy or light chain variable domains according to the invention described herein, wherein the antibody or antigen binding fragment thereof is rat, mouse, primate (e.g. cynomolgus, Old World monkey or Great Ape) or human.

In another embodiment of the present invention the antibody or antigen binding fragment thereof additionally binds primate IGF-1R, for example cynomolgus macaque monkey IGF-1R.

In another embodiment of the present invention there is provided an antibody or antigen binding fragment thereof comprising one or more of the following CDR's: CDRH3 as set out in as set out in SEQ. ID. NO: 1, CDRH2 as set out in SEQ. ID. NO: 2, CDRH1 as set out in SEQ. ID. NO: 3, CDRL1 as set out in SEQ. ID. NO: 4, CDRL2 as set out in SEQ. ID. NO: 5 and CDRL3 as set out in SEQ. ID. NO: 6 in the context of a human framework, for example as a humanised or chimaeric antibody.

In one embodiment of the present invention the humanised heavy chain variable domain comprises the CDR's listed in SEQ ID NO: 1-3 within an acceptor antibody framework having greater than 80% identity in the framework regions, or greater than 85%, or greater than 90%, or greater than 95%, or greater than 98%, or greater than 99% identity in the framework regions to the human acceptor sequence in SEQ ID NO: 59

In one embodiment of the present invention the humanised light chain variable domain comprises the CDR's listed in SEQ ID NO: 4-6 within an acceptor antibody framework having greater than 80% identity in the framework regions, or greater than 85%, or greater than 90%, or greater than 95%, or greater than 98%, or greater than 99% identity in the framework regions to the human acceptor sequence in SEQ ID NO: 60

In SEQ ID NO: 59 and SEQ ID NO: 60 the position of the CDR sequences have been denoted by Xaa's.

In another embodiment of the present invention there is provided an antibody or antigen binding fragment thereof comprising CDR's according to the invention described herein, or heavy or light chain variable domains according to the invention described wherein the antibody further comprises a constant region, which may be of any isotype or subclass. In one embodiment the heavy chain constant region is of the IgG isotype, for example IgG1, IgG2, IgG3, IgG4 or variants thereof. In one embodiment the antibody is IgG1.

In one embodiment of the present invention there is provided an antibody according to the invention described herein and comprising a constant region such that the antibody has reduced ADCC and/or complement activation or effector functionality. In one such embodiment the heavy chain constant region may comprise a naturally disabled constant region of IgG2 or IgG4 isotype or a mutated IgG1 constant region. Examples of suitable modifications are described in EP0307434. One example comprises the substitutions of alanine residues at positions 235 and 237 (EU index numbering).

In another embodiment of the present invention there is provided an antibody according to the invention described herein wherein the antibody is capable of at least some effector function for example wherein it is capable of some ADCC and/or CDC function. In one embodiment of the present invention there is provided an antibody comprising a constant region or antigen binding fragment thereof which is linked to a constant region which specifically binds IGF-1R, for example human IGF-1R comprising CDR H3 of SEQ. ID. NO: 1 or variant thereof which contains 1 or 2 amino acid substitutions in the CDRH3, for example an antibody comprising a constant region or antigen binding fragment thereof which is linked to a constant region comprising CDR's selected from CDRH1: SEQ. ID. NO: 3, CDRH2: SEQ. ID. NO: 2, CDRH3: SEQ. ID. NO: 1, CDRL1: SEQ. ID. NO: 4, CDRL2: SEQ. ID. NO: 7 and CDRL3: SEQ. ID. NO: 6, and which further comprises a constant region of IgG1 wild type, IgG2 wild type, IgG3 wild type, IgG4 wild type or enhanced versions thereof.

In one embodiment of the present invention the antibody comprising a constant region or antigen binding fragment thereof which is linked to a constant region, specifically binds to a growth factor receptor selected from IGF-1R, EGFR, HER-2 or HER-3. For example which specifically binds to HER-2 or HER-3 or for example which specifically binds to IGF-1R or EGRF, for example human IGF-1R.

In one embodiment of the present invention there is provided an antibody or antigen binding fragment thereof according to the invention described herein which comprises one or more mutations in its heavy chain constant region such that the antibody or antigen binding fragment has enhanced effector function. For example, wherein it has enhanced ADCC or enhanced CDC or wherein it has both enhanced ADCC and CDC effector function. Examples of suitable modifications are described in Shields et al. J. Biol. Chem. (2001) 276:6591-6604, Lazar et al. PNAS (2006) 103:4005-4010 and U.S. Pat. No. 6,737,056, WO2004063351 and WO2004029207.

In one embodiment of the present invention there is provided an antibody comprising a heavy chain constant region or antigen binding fragment thereof which is linked to a heavy chain constant region which specifically binds IGF-1R, for example human IGF-1R. The antibody or antigen binding fragment thereof may comprise CDR H3 of SEQ. ID. NO: 1 or variants thereof in which one or two amino acid residues within CDR H3 differ from the amino acid residues in the corresponding position in SEQ. ID. NO: 1 and comprising a mutated heavy chain constant region such that the antibody or antigen binding fragment thereof has enhanced effector function compared to wild type. For example, an antibody or antigen binding fragment thereof which specifically binds IGF-1R comprising CDR H3 of SEQ. ID. NO: 1, for example an antibody or antigen binding fragment thereof comprising CDR's selected from CDRH1: SEQ. ID. NO: 3, CDRH2: SEQ. ID. NO: 2, CDRH3: SEQ. ID. NO: 1, CDRL1: SEQ. ID. NO: 4, CDRL2: SEQ. ID. NO: 7 and CDRL3: SEQ. ID. NO: 6 and comprising a mutated heavy chain constant region such that the antibody or antigen binding fragment thereof has enhanced effector function compared to wild type.

In one embodiment of the present invention, such mutations are in one or more of positions selected from 239, 332 and 330 (IgG1), or the equivalent positions in other IgG isotypes. Examples of suitable mutations are S239D and I332E and A330L. In one embodiment the antibody or antigen binding fragment is mutated at positions 239 and 332, for example S239D and I332E, for example it is mutated at three or more positions selected from 239 and 332 and 330, for example S239D and I332E and A330L.

In another embodiment of the present invention there is provided an antibody comprising a heavy chain constant region or antigen binding fragment thereof which is linked to a heavy chain constant region according to the invention described herein and comprising a constant region selected from those set out in SEQ ID NO: 64 and SEQ ID. NO: 66, for example an antibody or antigen binding fragment comprising the variable domains of SEQ ID NO: 14 and SEQ ID NO: 15 together with the heavy chain constant region as set out in SEQ ID NO: 64 or SEQ ID NO: 66, for example an antibody comprising a heavy chain constant region or antigen binding fragment thereof which is linked to a heavy chain constant region comprising SEQ ID NO: 14, SEQ ID NO: 15 and SEQ ID NO: 64. In a further embodiment of the present invention there is provided an antibody comprising a heavy chain constant region or antigen binding fragment thereof which is linked to a heavy chain constant region according to the invention described herein and comprising a heavy chain constant region selected from those set out in SEQ ID NO: 64 and SEQ ID. NO: 66, for example antibody or antigen binding fragment thereof comprising the variable domains of SEQ ID NO: 14 and SEQ ID NO: 16 together with the heavy chain constant region as set out in SEQ ID NO: 64 or SEQ ID NO: 66, for example an antibody or antigen binding fragment thereof comprising SEQ ID NO: 14, SEQ ID NO: 16 and SEQ ID NO: 64.

In one embodiment of the present invention there is provided an antibody comprising a heavy chain constant region or antigen binding fragment thereof which is linked to a heavy chain constant region according to the invention described herein which comprises a heavy chain constant region with an altered glycosylation profile such that the antibody or antigen binding fragment thereof has enhanced effector function. For example, wherein it has enhanced ADCC or enhanced CDC or wherein it has both enhanced ADCC and CDC effector function. Examples of suitable methodologies to produce antibodies with an altered glycosylation profile are described in WO2003011878, WO2006014679 and EP1229125.

In one embodiment of the present invention there is provided an antibody comprising a heavy chain constant region or antigen binding fragment thereof which is linked to a heavy chain constant region according to the invention described herein which specifically binds IGF-1R, for example human IGF-1R. The antibody or antigen binding fragment thereof may comprise CDR H3 of SEQ. ID. NO: 1 or variants thereof in which one or two amino acid residues within CDR H3 differ from the amino acid residues in the corresponding position in SEQ. ID. NO: 1 and comprising a heavy chain constant region with an altered glycosylation profile such that the antibody or antigen binding fragment has enhanced effector function when compared to wild type.

For example, an antibody or antigen binding fragment thereof which specifically binds IGF-1R, for example human IGF-1R comprising CDR H3 of SEQ. ID. NO: 1, for example an antibody or antigen binding fragment thereof comprising CDR's selected from CDRH1: SEQ. ID. NO: 3, CDRH2: SEQ. ID. NO: 2, CDRH3: SEQ. ID. NO: 1, CDRL1: SEQ. ID. NO: 4, CDRL2: SEQ. ID. NO: 7 and CDRL3: SEQ. ID. NO: 6 and comprising a heavy chain constant region with an altered glycosylation profile such that the antibody or antigen binding fragment has enhanced effector function when compared to wild type.

In one embodiment the invention provides an antibody preparation wherein the ratio of fucose to mannose in said antibody preparation is 0.8:3 or less, for example is 0.7:3 or less, or is 0.6:3 or less or is 0.5:3 or less or is 0.4:3 or less or is 0.3:3 or less, or is 0.2:3 or less or is 0.1:3 or less. In one embodiment the antibody preparation contains negligible or no bound fucose.

In another embodiment of the present invention there is provided an antibody preparation comprising an antibody or antigen binding fragment thereof comprising the variable domains of SEQ ID NO: 14 and SEQ ID NO: 15 or SEQ ID NO: 14 and SEQ ID NO: 16 and wherein the ratio of fucose to mannose in said antibody preparation is 0.8:3 or less, for example is 0.7:3 or less, or is 0.6:3 or less or is 0.5:3 or less or is 0.4:3 or less or is 0.3:3 or less, or is 0.2:3 or less or is 0.1:3 or less. In one embodiment the antibody preparation contains negligible or no bound fucose.

In one embodiment of the present invention there is provided an antibody comprising a heavy chain constant region or antigen binding fragment thereof which is linked to a heavy chain constant region according to the invention described herein which comprises a mutated heavy chain constant region and an altered glycosylation profile such that the antibody or antigen binding fragment has enhanced effector function, for example wherein it has one or more of the following functions, enhanced ADCC or enhanced CDC, for example wherein it has enhanced ADCC function.

In one embodiment of the invention antibody preparation comprising antibodies which comprise an immunoglobulin heavy chain constant region, or antigen binding fragments thereof which are linked to an immunoglobulin heavy chain constant region wherein said immunoglobulin heavy chain constant region confers an effector function to the antibody or antigen binding fragment, and wherein said antibody or antigen binding fragment specifically binds to a growth factor receptor and wherein said immunoglobulin heavy chain constant region is mutated in at least 2 positions and has an altered glycosylation profile such that the ratio of fucose to mannose is 0.8:3 or less so that said antibody or antigen binding fragment has an enhanced effector function in comparison with an equivalent antibody or antigen-binding fragment with an immunoglobulin heavy chain constant region lacking said mutations and altered glycosylation profile. The altered glycosylation profile of said antibody preparation is not a consequence of said immunoglobulin heavy chain mutations.

For example, such antibodies or antigen binding fragments specifically bind IGF-1R, for example human IGF-1R and comprise CDR H3 of SEQ. ID. NO: 1, for example an antibody or antigen binding fragment comprising CDR's selected from CDRH1: SEQ. ID. NO: 3, CDRH2: SEQ. ID. NO: 2, CDRH3: SEQ. ID. NO: 1, CDRL1: SEQ. ID. NO: 4, CDRL2: SEQ. ID. NO: 7 and CDRL3: SEQ. ID. NO: 6 and comprise a mutated heavy chain constant region and have an altered glycosylation profile such that the antibody or antigen binding fragment has enhanced effector function. For example such antibodies or antigen binding fragments may comprise the variable domains of SEQ ID NO: 14 and SEQ ID NO: 15 or SEQ ID NO: 14 and SEQ ID NO: 16.

In one such embodiment, the mutations are in one or more of positions selected from 239, 332 and 330 (IgG1), or the equivalent positions in other IgG isotypes. Examples of suitable mutations are S239D and I332E and A330L. In one embodiment the antibody comprising a constant region or antigen binding fragment thereof which is linked to a constant region has a mutation at 239 and 332, for example S239D and I332E or further may comprise mutations at three or more positions selected from 239 and 332 and 330, for example S239D and I332E and A330L.

In one embodiment the ratio of fucose to mannose in said antibody preparation is 0.8:3 or less, for example is 0.7:3 or less, or is 0.6:3 or less or is 0.5:3 or less or is 0.4:3 or less or is 0.3:3 or less, or is 0.2:3 or less or is 0.1:3 or less. In one embodiment the antibody preparation contains negligible or no bound fucose.

In one embodiment of the present invention there is provided an antibody comprising a heavy chain constant region or antigen binding fragment thereof which is linked to a heavy chain constant region according to the invention described herein which comprises a chimaeric heavy chain constant region for example wherein it comprises at least one CH2 domain from IgG3 such that the antibody or antigen binding fragment has enhanced effector function, for example wherein it has one or more of the following functions, enhanced ADCC or enhanced CDC, for example wherein it has enhanced CDCC. For example the antibody or antigen binding fragment may comprise one CH2 domain from IgG3 or both CH2 domains may be from IgG3.

In a further embodiment of the present invention there is provided an antibody comprising a heavy chain constant region or antigen binding fragment thereof which is linked to a heavy chain constant region according to the invention described herein which comprises a mutated and chimaeric heavy chain constant region for example wherein it comprises at least one CH2 domain from IgG3 and one CH2 domain from IgG1 wherein the IgG1 CH2 domain has one or more mutations at positions selected from 239 and 332 and 330, for example the mutations are selected from S239D and I332E and A330L such that the antibody has enhanced effector function, for example wherein it has one or more of the following functions, enhanced ADCC or enhanced CDC, for example wherein it has enhanced ADCC and enhanced CDCC. In one embodiment the IgG1 CH2 domain has the mutations S239D and I332E.

In one embodiment of the present invention there is provided an antibody comprising a heavy chain constant region or antigen binding fragment thereof which is linked to a heavy chain constant region according to the invention described herein which comprises a chimaeric heavy chain constant region and an altered glycosylation profile such that the heavy chain constant region comprises at least one CH2 domain from IgG3 and one CH2 domain from IgG1 and which has an altered glycosylation profile such that the ratio of fucose to mannose is 0.8:3 or less so that said antibody or antigen binding fragment has an enhanced effector function in comparison with an equivalent antibody or antigen-binding fragment with an immunoglobulin heavy chain constant region lacking said mutations and altered glycosylation profile, such that the antibody or antigen binding fragment has enhanced effector function, for example wherein it has one or more of the following functions, enhanced ADCC or enhanced CDC, for example wherein it has enhanced ADCC and enhanced CDCC.

In an alternative embodiment the antibody or antigen binding fragment has at least one IgG3 CH2 domain and at least one heavy chain constant domain from IgG1 wherein both IgG CH2 domains are mutated in accordance with the limitations described herein.

In one embodiment of the present invention there is provided an antibody preparation comprising an antibody comprising a heavy chain constant region or antigen binding fragment thereof which is linked to a heavy chain constant region which comprises a mutated and chimaeric heavy chain constant region wherein said antibody preparation has an altered glycosylation profile such that the antibody or antigen binding fragment has enhanced effector function, for example wherein it has one or more of the following functions, enhanced ADCC or enhanced CDC. In one embodiment the mutations are selected from positions 239 and 332 and 330, for example the mutations are selected from S239D and I332E and A330L. In a further embodiment the heavy chain constant region comprises at least one CH2 domain from IgG3 and one Ch2 domain from IgG1. In one embodiment the heavy chain constant region has an altered glycosylation profile such that the ratio of fucose to mannose is 0.8:3 or less so that said antibody or antigen binding fragment has an enhanced effector function in comparison with an equivalent non-chimaeric antibody or antigen-binding fragment thereof with an immunoglobulin heavy chain constant region lacking said mutations and altered glycosylation profile.

In one embodiment of the present invention there is provided a recombinant transformed, transfected or transduced host cell comprising at least one expression cassette, for example where the expression cassette comprises a polynucleotide encoding a heavy chain of an antibody or antigen binding fragment thereof according to the invention described herein and further comprises a polynucleotide encoding a light chain of a antibody or antigen binding fragment thereof according to the invention described herein or where there are two expression cassettes and the 1^{st} encodes the light chain and the second encodes the heavy chain. For example in one embodiment the first expression cassette comprises a polynucleotide encoding a heavy chain of an antibody comprising a constant region or antigen binding fragment thereof which is linked to a constant region according to the invention described herein and further comprises a second cassette comprising a polynucleotide encoding a light chain of an antibody comprising a constant region or antigen binding fragment thereof which is linked to a constant region according to the invention described herein for example the first expression cassette comprises a polynucleotide encoding a heavy chain selected from SEQ. ID. NO: 40, SEQ. ID. NO: 41 or SEQ. ID. NO: 67 or SEQ. ID. NO: 70 and a second expression cassette comprising a polynucleotide encoding a light chain selected from SEQ. ID. NO: 42 or SEQ. ID. NO: 69.

In another embodiment of the invention there is provided a stably transformed host cell comprising a vector comprising one or more expression cassettes encoding a heavy chain and/or a light chain of the antibody comprising a constant region or antigen binding fragment thereof which is linked to a constant region as described herein. For example such host cells may comprise a first vector encoding the light chain and a second vector encoding the heavy chain, for example the first vector encodes a heavy chain selected from SEQ. ID. NO: 37, SEQ. ID. NO: 38 or SEQ. ID. NO: 68 and a second vector encoding a light chain for example the light chain of SEQ ID NO: 39.

In another embodiment of the present invention there is provided a host cell according to the invention described herein wherein the cell is eukaryotic, for example where the cell is mammalian. Examples of such cell lines include CHO or NS0.

In another embodiment of the present invention there is provided a method for the production of an antibody comprising a constant region or antigen binding fragment thereof which is linked to a constant region according to the invention described herein which method comprises the step of culturing a host cell in a culture media, for example serum-free culture media.

In another embodiment of the present invention there is provided a method according to the invention described herein wherein said antibody is further purified to at least 95% or greater (e.g. 98% or greater) with respect to said antibody containing serum-free culture media.

In another embodiment of the present invention there is provided a pharmaceutical composition comprising an antibody comprising a constant region or antigen binding fragment thereof which is linked to a constant region according to the invention described herein and a pharmaceutically acceptable carrier.

In another embodiment of the present invention there is provided a kit-of-parts comprising the composition according to the invention described herein described together with instructions for use.

In another embodiment of the present invention there is provided a method of treating a human patient afflicted with rheumatoid arthritis which method comprises the step of administering a therapeutically effective amount of the antibody comprising a constant region or antigen binding fragment thereof which is linked to a constant region according to the invention described herein. The antibody comprising a constant region or antigen binding fragment thereof which is linked to a constant region may be in combination with a pharmaceutically acceptable carrier.

In another embodiment of the present invention there is provided a method of treating a human patient afflicted with cancer which method comprises the step of administering a therapeutically effective amount of antibody comprising a constant region or antigen binding fragment thereof which is linked to a constant region according to the invention described herein. The antibody comprising a constant region or antigen binding fragment thereof which is linked to a constant region may be in combination with a pharmaceutically acceptable carrier.

In a further embodiment of the present invention there is provided a method of treating a human patient afflicted with cancer which method comprises the step of administering a therapeutically effective amount of the pharmaceutical composition comprising an antibody comprising a constant region or antigen binding fragment thereof which is linked to a constant region according to the invention described herein and a pharmaceutically acceptable carrier.

In another embodiment of the present invention there is provided use of an antibody comprising a constant region or antigen binding fragment thereof which is linked to a constant region according to the invention described herein in the manufacture of a medicament for the treatment of a disease or disorder selected from the group consisting of; Rheumatoid arthritis, Psoriasis or Cancers for example: Acute Lymphoblastic Leukemia, Adrenocortical Carcinoma, AIDS-Related Cancers, AIDS Related Lymphoma, Anal Cancer, Childhood Cerebellar Astrocytoma, Childhood Cerebral Astrocytoma, Colorectal Cancer, Basal Cell Carcinoma, Extrahepatic Bile Duct Cancer, Bladder Cancer, Osteosarcorna/Malignant Fibrous Histiocytoma Bone Cancer, Brain Tumors (e.g., Brain Stem Glioma, Cerebellar Astrocytoma, Cerebral Astrocytoma/Malignant Glioma, Ependymoma, Medulloblastoma, Supratentorial Primitive Neuroectodermal Tumors, Visual Pathway and Hypothalamic Glioma), Breast Cancer, Bronchial Adenomas/Carcinoids, Burkitt's Lymphoma, Carcinoid Tumor, Gastrointestinal Carcinoid Tumor, Carcinoma of Unknown Primary, Primary Central Nervous System, Cerebellar Astrocytoma, Cerebral Astrocytoma/Malignant Glioma, Cervical Cancer, Childhood Cancers, Chronic Lymphocytic Leukemia, Chronic Myelogenous Leukemia, Chronic Myeloproliferative Disorders, Colon Cancer, Colorectal Cancer, Cutaneous T-Cell Lymphoma, Endometrial Cancer, Ependymoma, Esophageal Cancer, Ewing's Family of Tumors, Extracranial Germ Cell Tumor, Extragonadal Germ Cell Tumor, Extrahepatic Bile Duct Cancer, Intraocular Melanoma Eye Cancer, Retinoblastoma Eye Cancer, Gallbladder Cancer, Gastric (Stomach) Cancer, Gastrointestinal Carcinoid Tumor, Germ Cell Tumors (e.g., Extracranial, Extragonadal, and Ovarian), Gestational Trophoblastic Tumor, Glioma (e.g., Adult, Childhood Brain Stem, Childhood Cerebral Astrocytoma, Childhood Visual Pathway and Hypothalamic), Hairy Cell Leukemia, Head and Neck Cancer, Hepatocellular (Liver) Cancer, Hodgkin's Lymphoma, Hypopharyngeal Cancer, Hypothalamic and Visual Pathway Glioma, Intraocular Melanoma, Islet Cell Carcinoma (Endocrine Pancreas), Kaposi's Sarcoma, Kidney (Renal Cell) Cancer, Laryngeal Cancer, Leukemia (e.g., Acute Lymphoblastic, Acute Myeloid, Chronic Lymphocyhc, Chronic Myelogenous, and Hairy Cell), Lip and Oral Cavity Cancer, Liver Cancer, Non-Small Cell Lung Cancer, Small Cell Lung Cancer, Lymphoma (e.g., AIDS-Related, Burkitt's, Cutaneous T-cell, Hodgkin's, Non-Hodgkin's, and Primary Central Nervous System), Waldenstrom's Macroglobulinemia, Malignant Fibrous Histiocytoma of Bone/Osteosarcoma, Medulloblastoma, Melanoma, Intraocular (Eye) Melanoma, Merkel Cell Carcinoma, Mesothelioma, Metastatic Squamous Neck Cancer with Occult Primary, Multiple Endocrine Neoplasia Syndrome, Multiple Myeloma/Plasma Cell Neoplasm, Mycosis Fungoides, Myelodysplastic Syndromes, Myelodysplastic/Myeloproliferative Diseases, Myelogenous Leukemia, Chronic Myeloid Leukemia, Multiple Myeloma, Chronic Myeloproliferative Disorders, Nasal Cavity and Paranasal Sinus Cancer, Nasopharyngeal Cancer, Neuroblastoma, Oral Cancer, Oropharyngeal Cancer, Osteosarcoma/Malignant Fibrous Histiocytoma of Bone, Ovarian Cancer, Ovarian Epithelial Cancer, Ovarian Germ Cell Tumor, Ovarian Low Malignant Potential Tumor, Pancreatic Cancer, Islet Cell Pancreatic Cancer, Paranasal Sinus and Nasal Cavity Cancer, Parathyroid Cancer, Penile Cancer, Pheochromocytoma, Pineoblastoma, Pituitary Tumor, Plasma Cell Neoplasm/Multiple Myeloma, Pleuropulmonary Blastoma, Primary Central Nervous System Lymphoma, Prostate Cancer, Rectal Cancer, Renal Cell (Kidney) Cancer, Renal Pelvis and Ureter Transitional Cell Cancer, Retinoblastoma, Rhabdomyosarcoma, Salivary Gland Cancer, Soft Tissue Sarcoma, Uterine Sarcoma, Sezary Syndrome, non-Melanoma Skin Cancer, Merkel Cell Skin Carcinoma, Small Intestine Cancer, Soft Tissue Sarcoma, Squamous Cell Carcinoma, Cutaneous T-cell Lymphoma, Testicular Cancer, Thyrnoma, Thymic Carcinoma, Thyroid Cancer, Gestational Trophoblastic Tumor, Carcinoma of Unknown Primary Site, Cancer of Unknown Primary Site, Urethral Cancer, Endometrial Uterine Cancer, Uterine Sarcoma, Vaginal Cancer, Visual Pathway and Hypothalamic Glioma, Vulvar Cancer, Waldenstrom's Macroglobulinemia, and Wilms' Tumor.

In another embodiment of the present invention there is provided a method according to the invention described herein wherein the patient is afflicted with one or more of: Rheumatoid Arthritis, Psoriasis, Colorectal Cancer, Breast Cancer, Prostate Cancer, Lung Cancer or Myeloma

### DEFINITIONS

The term “antibody” is used herein in the broadest sense and specifically covers monoclonal antibodies (including full length monoclonal antibodies), polyclonal antibodies, multispecific antibodies (e.g. bispecific antibodies), and antibody fragments so long as they exhibit the desired biological activity. These are explained later in further detail.

The term “monoclonal antibody” as used herein refers to an antibody obtained from a population of substantially homogenous antibodies i.e. the individual antibodies comprising the population are identical except for possible naturally occurring mutations that may be present in minor amounts. Monoclonal antibodies are highly specific being directed against a single antigenic binding site. Furthermore, in contrast to polyclonal antibody preparations which typically include different antibodies directed against different determinants (epitopes), each monoclonal antibody is directed against a single determinant on the antigen.

“Identity,” means, for polynucleotides and polypeptides, as the case may be, the comparison calculated using an algorithm provided in (1) and (2) below:
(1) Identity for polynucleotides is calculated by multiplying the total number of nucleotides in a given sequence by the integer defining the percent identity divided by 100 and then subtracting that product from said total number of nucleotides in said sequence, or:

nn≦xn−(xn·y),

wherein nn is the number of nucleotide alterations, xn is the total number of nucleotides in a given sequence, y is 0.95 for 95%, 0.97 for 97% or 1.00 for 100%, and · is the symbol for the multiplication operator, and wherein any non-integer product of xn and y is rounded down to the nearest integer prior to subtracting it from xn. Alterations of a polynucleotide sequence encoding a polypeptide may create nonsense, missense or frameshift mutations in this coding sequence and thereby alter the polypeptide encoded by the polynucleotide following such alterations.
(2) Identity for polypeptides is calculated by multiplying the total number of amino acids by the integer defining the percent identity divided by 100 and then subtracting that product from said total number of amino acids, or:

na≦xa−(xa·y),

wherein na is the number of amino acid alterations, xa is the total number of amino acids in the sequence, y is 0.95 for 95%, 0.97 for 97% or 1.00 for 100%, and · is the symbol for the multiplication operator, and wherein any non-integer product of xa and y is rounded down to the nearest integer prior to subtracting it from xa.

The term “Variant(s)” as used herein, refers to a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusion proteins and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. It is well recognised in the art that certain amino acid substitutions are regarded as being “conservative”. Amino acids are divided into groups based on common side-chain properties and substitutions within groups that maintain all or substantially all of the binding affinity of the antibody of the invention or antigen binding fragment thereof are regarded as conservative substitutions, see table below:

| Side chain | Members |
|---|---|
| Hydrophobic | met, ala, val, leu, ile |
| neutral hydrophilic | cys, ser, thr |
| Acidic | asp, glu |
| Basic | asn, gln, his, lys, arg |
| residues that influence chain orientation | gly, pro |
| Aromatic | trp, tyr, phe |

In some aspects of the invention variants in which several, for example 5-10, 1-5, 1-3, 1-2 amino acid residues or 1 amino acid residue are substituted, deleted, or added in any combination may be included. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

“Isolated” means altered “by the hand of man” from its natural state, has been changed or removed from its original environment, or both. For example, a polynucleotide or a polypeptide naturally present in a living organism is not “isolated,” but the same polynucleotide or polypeptide separated from the coexisting materials of its natural state is “isolated”, including but not limited to when such polynucleotide or polypeptide is introduced back into a cell, even if the cell is of the same species or type as that from which the polynucleotide or polypeptide was separated.

Throughout the present specification and the accompanying claims the term “comprising” and “comprises” incorporates “consisting of” and “consists of”. That is, these words are intended to convey the possible inclusion of other elements or integers not specifically recited, where the context allows.

The term “glycosylation profile” as used herein refers to the levels of glycosylation in an antibody population.

The term “specifically binds” as used throughout the present specification in relation to antibodies and antigen binding fragments thereof of the invention means that the antibody binds human IGF-1R (hIGF-1R) with no or insignificant binding to other human proteins. The term however does not exclude the fact that antibodies of the invention may also be cross-reactive with other forms of IGF-1R, for example primate IGF-1R.

The term “neutralises” as used throughout the present specification in relation to antibodies and antigen binding fragments thereof of the invention means that the biological activity of IGF-1R is reduced in the presence of the antibodies and antigen binding fragments thereof of the present invention in comparison to the activity of IGF-1R in the absence of such antibodies and antigen binding fragments thereof. Neutralisation may be due to but not limited to one or more of blocking ligand binding, preventing the ligand activating the receptor, down regulating the IGF-1R or affecting effector functionality. Levels of neutralisation can be measured in several ways, for example by use of the assays as set out in the examples below, for example in a LISN cell proliferation assay which may be carried out for example as described in Example 23. The neutralisation of IGF-1R in this assay is measured by assessing the decreased tumour cell proliferation in the presence of neutralising antibody.

Levels of neutralisation can also be measured, for example in a receptor phosphorylation assay which may be carried out for example as described in Example 13. The neutralisation of IGF-1R in this assay is measured by assessing the inhibition of receptor phosphorylation in the presence of neutralising antibody.

If an antibody or antigen binding fragment thereof is capable of neutralisation then this is indicative of inhibition of the interaction between human IGF-1R binding proteins for example hIGF-I or hIGF-II and its receptor. Antibodies which are considered to have neutralising activity against human IGF-1R would have an IC₅₀ of less than 10 micrograms/ml, or less than 5 micrograms/ml, or less than 2 micrograms/ml, or less than 1 microgram/ml in the LISN cell proliferation assay or receptor phosphorylation assay as set out in Examples 23 and Example 13 respectively.

In an alternative aspect of the present invention there is provided antibodies or antigen binding fragments thereof which have equivalent neutralising activity to the antibodies exemplified herein, for example antibodies which retain the neutralising activity of H0L0 and H0L0 IgG1m(AA) and H1L0 and H10L0 IgG1m(AA) in the LISN cell proliferation assay or receptor phosphorylation assay as set out in Examples 23 and 13 respectively.

Throughout this specification, amino acid residues in antibody sequences are numbered according to the Kabat scheme. Similarly, the terms “CDR”, “CDRL1”, “CDRL2”, “CDRL3”, “CDRH1”, “CDRH2”, “CDRH3” follow the Kabat numbering system as set forth in Kabat et al; Sequences of proteins of Immunological Interest NIH, 1987. It will be apparent to those skilled in the art that there are alternative definitions of CDR sequences such as for example those set out in Chothia et al. (1989).

It will be apparent to those skilled in the art that the term “derived” is intended to define not only the source in the sense of it being the physical origin for the material but also to define material which is structurally identical (in terms of primary amino acid sequence) to the material but which does not originate from the reference source. Thus “residues found in the donor antibody from which CDRH3 is derived” need not necessarily have been purified from the donor antibody.

A “chimeric antibody” refers to a type of engineered antibody which contains a naturally-occurring variable domain (light chain and heavy chains) derived from a donor antibody in association with light and heavy chain constant regions derived from an acceptor antibody.

A “humanised antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., Queen et al., Proc. Natl. Acad Sci USA, 86:10029-10032 (1989), Hodgson et al., Bio/Technology, 9:421 (1991)). A suitable human acceptor antibody may be one selected from a conventional database, e.g., the KABAT® database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanised antibodies—see for example EP-A-0239400 and EP-A-054951.

The term “donor antibody” refers to an antibody (monoclonal, and/or recombinant) which contributes the amino acid sequences of its variable domains, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but preferably all) of the amino acid sequences encoding its heavy and/or light chain framework regions and/or its heavy and/or light chain constant regions to the first immunoglobulin partner. The human antibody is the acceptor antibody.

“CDRs” are defined as the complementarity determining region amino acid sequences of an antibody which are the hypervariable domains of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable domains; Nature 342, p 877-883.

CDRs provide the majority of contact residues for the binding of the antibody to the antigen or epitope. CDRs of interest in this invention are derived from donor antibody variable heavy and light chain sequences, and include analogs of the naturally occurring CDRs, which analogs also share or retain the same antigen binding specificity and/or neutralizing ability as the donor antibody from which they were derived.

The terms “V_{H}” and “V₆” are used herein to refer to the heavy chain variable domain and light chain variable domain respectively of an antibody.

The term “Effector Function” as used herein is meant to refer to one or more of Antibody dependant cell mediated cytotoxic activity (ADCC) and complement-dependant cytotoxic activity (CDC) mediated responses, Fc-mediated phagocytosis and antibody recycling via the FcRn receptor. The interaction between the constant region of an antibody and various Fc receptors (FcR) is believed to mediate the effector functions of the antibody. Significant biological effects can be a consequence of effector functionality, in particular, antibody-dependent cellular cytotoxicity (ADCC), fixation of complement (complement dependent cytotoxicity or CDC), phagocytosis (antibody-dependent cell-mediated phagocytosis or ADCP) and half-life/clearance of the antibody. Usually, the ability to mediate effector function requires binding of the antibody to an antigen and not all antibodies will mediate every effector function.

Effector function can be measured in a number of ways including for example via binding of the FcγRIII to Natural Killer cells or via FcγRI to monocytes/macrophages to measure for ADCC effector function. For example the antibody or antigen binding fragment of the present invention has an increased ADCC effector function when measured against the equivalent wild type antibody or antigen binding fragment thereof in a Natural Killer cell assay. Examples of such assays can be found in Shields et al, 2001 The Journal of Biological Chemistry, Vol. 276, p 6591-6604; Chappel et al, 1993 The Journal of Biological Chemistry, Vol 268, p 25124-25131; Lazar et al, 2006 PNAS, 103; 4005-4010.

Examples of assays to determine CDC function include that described in 1995 J Imm Meth 184:29-38.

Various modifications to the heavy chain constant region of antibodies may be carried out depending on the desired effector property. Human constant regions which essentially lack the functions of a) activation of complement by the classical pathway; and b) mediating antibody-dependent cellular cytotoxicity include the IgG4 constant region and the IgG2 constant region. IgG1 constant regions containing specific mutations have separately been described to reduce binding to Fc receptors and therefore reduce ADCC and CDC (Duncan et al. Nature 1988, 332; 563-564; Lund et al. J. Immunol. 1991, 147; 2657-2662; Chappel et al. PNAS 1991, 88; 9036-9040; Burton and Woof, Adv. Immunol. 1992, 51; 1-84; Morgan et al., Immunology 1995, 86; 319-324; Hezareh et al., J. Virol. 2001, 75 (24); 12161-12168). Human IgG1 constant regions containing specific mutations or altered glycosylation on residue Asn297 have also been described to enhance binding to Fc receptors. These have also been shown to enhance ADCC and CDC, in some cases (Lazar et al. PNAS 2006, 103; 4005-4010; Shields et al. J Biol Chem 2001, 276; 6591-6604; Nechansky et al. Mol Immunol, 2007, 44; 1815-1817).

For IgG antibodies, effector functionalities including ADCC and ADCP are mediated by the interaction of the heavy chain constant region with a family of Fcγ receptors present on the surface of immune cells. In humans these include FcγRI (CD64), FcγRII (CD32) and FcγRIII (CD16). Interaction between the antibody bound to antigen and the formation of the Fc/Fcγ complex induces a range of effects including cytotoxicity, immune cell activation, phagocytosis and release of inflammatory cytokines. Specific substitutions in the constant region (including S239D/I332E) are know to increase the affinity of the heavy chain constant region for certain Fc receptors, thus enhancing the effector functionality of the antibody (Lazar et al. PNAS 2006).

### 1. Antibody Structures

### 1.1 Intact Antibodies

Intact antibodies include heteromultimeric glycoproteins comprising at least two heavy and two light chains. Aside from IgM, intact antibodies are usually heterotetrameric glycoproteins of approximately 150 Kda, composed of two identical light (L) chains and two identical heavy (H) chains. Typically, each light chain is linked to a heavy chain by one covalent disulfide bond while the number of disulfide linkages between the heavy chains of different immunoglobulin isotypes varies. Each heavy and light chain also has intrachain disulfide bridges. Each heavy chain has at one end a variable domain (V_{H}) followed by a number of constant regions (CH₁, CH₂, CH₃). Each light chain has a variable domain (V_{L}) and a constant region at its other end; the heavy chain constant region of the light chain is aligned with the first constant region of the heavy chain and the light chain variable domain is aligned with the variable domain of the heavy chain. The light chains of antibodies from most vertebrate species can be assigned to one of two types called Kappa and Lambda based on the amino acid sequence of the constant region. Depending on the amino acid sequence of the heavy chain constant region of their heavy chains, human antibodies can be assigned to five different classes, IgA, IgD, IgE, IgG and IgM. IgG and IgA can be further subdivided into subclasses, IgG1, IgG2, IgG3 and IgG4; and IgA1 and IgA2. Species variants exist with mouse and rat having at least IgG2a, IgG2b. The variable domain of the antibody confers binding specificity upon the antibody with certain regions displaying particular variability called complementarity determining regions (CDRs). The more conserved portions of the variable domain are called Framework regions (FR). The variable domains of intact heavy and light chains each comprise four FR connected by three CDRs. The CDRs in each chain are held together in close proximity by the FR regions and with the CDRs from the other chain contribute to the formation of the antigen binding site of antibodies. The constant regions are not directly involved in the binding of the antibody to the antigen but exhibit various effector functions such as participation in antibody dependent cell-mediated cytotoxicity (ADCC), phagocytosis via binding to Fcγ receptor, half-life/clearance rate via neonatal Fc receptor (FcRn) and complement dependent cytotoxicity via the C1q component of the complement cascade.

### 1.1.2 Human Antibodies

Human antibodies may be produced by a number of methods known to those of skill in the art. Human antibodies can be made by the hybridoma method using human myeloma or mouse-human heteromyeloma cells lines see Kozbor J. Immunol 133, 3001, (1984) and Brodeur, Monoclonal Antibody Production Techniques and Applications, pp 51-63 (Marcel Dekker Inc, 1987). Alternative methods include the use of phage libraries or transgenic mice both of which utilize human variable domain repertories (see Winter G, (1994), Annu. Rev. Immunol 12, 433-455, Green L L (1999), J. Immunol. methods 231, 11-23).

Several strains of transgenic mice are now available wherein their mouse immunoglobulin loci has been replaced with human immunoglobulin gene segments (see Tomizuka K, (2000) PNAS 97, 722-727; Fishwild D. M (1996) Nature Biotechnol. 14, 845-851, Mendez M J, 1997, Nature Genetics, 15, 146-156). Upon antigen challenge such mice are capable of producing a repertoire of human antibodies from which antibodies of interest can be selected. Of particular note is the Trimera™ system (see Eren R et al, (1998) Immunology 93:154-161) where human lymphocytes are transplanted into irradiated mice, the Selected Lymphocyte Antibody System (SLAM, see Babcook et al, PNAS (1996) 93:7843-7848) where human (or other species) lymphocytes are effectively put through a massive pooled in vitro antibody generation procedure followed by deconvulated, limiting dilution and selection procedure and the Xenomouse II™ (Abgenix Inc). An alternative approach is available from Morphotek Inc using the Morphodoma™ technology.

Phage display technology can be used to produce human antibodies (and fragments thereof), see McCafferty; Nature, 348, 552-553 (1990) and Griffiths A D et al (1994) EMBO 13:3245-3260. According to this technique antibody variable domain genes are cloned in frame into either a major or minor coat of protein gene of a filamentous bacteriophage such as M13 or fd and displayed (usually with the aid of a helper phage) as functional antigen binding fragments thereof on the surface of the phage particle. Selections based on the functional properties of the antibody result in selection of the gene encoding the antibody exhibiting those properties. The phage display technique can be used to select antigen specific antibodies from libraries made from human B cells taken from individuals afflicted with a disease or disorder described above or alternatively from unimmunized human donors (see Marks; J. Mol. Bio. 222, 581-597, 1991). Where an intact human antibody is desired comprising a constant domain it is necessary to reclone the phage displayed derived fragment into a mammalian expression vectors comprising the desired constant regions and establishing stable expressing cell lines.

The technique of affinity maturation (Marks; Bio/technol 10, 779-783 (1992)) may be used to improve binding affinity wherein the affinity of the primary human antibody is improved by sequentially replacing the H and L chain variable domains with naturally occurring variants and selecting on the basis of improved binding affinities. Variants of this technique such as “epitope imprinting” are now also available see WO 93/06213. See also Waterhouse; Nucl. Acids Res 21, 2265-2266 (1993).

### 1.2 Chimaeric and Humanised Antibodies

The use of intact non-human antibodies in the treatment of human diseases or disorders carries with it the potential for the now well established problems of immunogenicity, that is the immune system of the patient may recognise the non-human intact antibody as non-self and mount a neutralising response. This is particularly evident upon multiple administration of the non-human antibody to a human patient. Various techniques have been developed over the years to overcome these problems and generally involve reducing the composition of non-human amino acid sequences in the intact antibody whilst retaining the relative ease in obtaining non-human antibodies from an immunised animal e.g. mouse, rat or rabbit. Broadly two approaches have been used to achieve this. The first are chimaeric antibodies, which generally comprise a non-human (e.g. rodent such as mouse) variable domain fused to a human constant region. Because the antigen-binding site of an antibody is localised within the variable domains the chimaeric antibody retains its binding affinity for the antigen but acquires the effector functions of the human constant region and are therefore able to perform effector functions such as described supra. Chimaeric antibodies are typically produced using recombinant DNA methods. DNA encoding the antibodies (e.g. cDNA) is isolated and sequenced using conventional procedures (e.g. by using oligonucleotide probes that are capable of binding specifically to genes encoding the H and L chains of the antibody of the invention. Hybridoma cells serve as a typical source of such DNA. Once isolated, the DNA is placed into expression vectors which are then transfected into host cells such as E. Coli, COS cells, CHO cells or myeloma cells that do not otherwise produce immunoglobulin protein to obtain synthesis of the antibody. The DNA may be modified by substituting the coding sequence for human L and H chains for the corresponding non-human (e.g. murine) H and L constant regions see e.g. Morrison; PNAS 81, 6851 (1984).

The second approach involves the generation of humanised antibodies wherein the non-human content of the antibody is reduced by humanizing the variable domains. Two techniques for humanisation have gained popularity. The first is humanisation by CDR grafting. CDRs build loops close to the antibody's N-terminus where they form a surface mounted in a scaffold provided by the framework regions. Antigen-binding specificity of the antibody is mainly defined by the topography and by the chemical characteristics of its CDR surface. These features are in turn determined by the conformation of the individual CDRs, by the relative disposition of the CDRs, and by the nature and disposition of the side chains of the residues comprising the CDRs. A large decrease in immunogenicity can be achieved by grafting only the CDRs of a non-human (e.g. murine) antibodies (“donor” antibodies) onto human framework (“acceptor framework”) and constant regions (see Jones et al (1986) Nature 321, 522-525 and Verhoeyen M et al (1988) Science 239, 1534-1536). However, CDR grafting per se may not result in the complete retention of antigen-binding properties and it is frequently found that some framework residues (sometimes referred to as “back mutations”) of the donor antibody need to be preserved in the humanised molecule if significant antigen-binding affinity is to be recovered (see Queen C et al (1989) PNAS 86, 10,029-10,033, Co, M et al (1991) Nature 351, 501-502). In this case, human variable domains showing the greatest sequence homology to the non-human donor antibody are chosen from a database in order to provide the human framework (FR). The selection of human FRs can be made either from human consensus or individual human antibodies. Where necessary key residues from the donor antibody are substituted into the human acceptor framework to preserve CDR conformations. Computer modelling of the antibody maybe used to help identify such structurally important residues, see WO99/48523.

Alternatively, humanisation maybe achieved by a process of “veneering”. A statistical analysis of unique human and murine immunoglobulin heavy and light chain variable domains revealed that the precise patterns of exposed residues are different in human and murine antibodies, and most individual surface positions have a strong preference for a small number of different residues (see Padlan E. A. et al; (1991) Mol. Immunol. 28, 489-498 and Pedersen J. T. et al (1994) J. Mol. Biol. 235; 959-973). Therefore it is possible to reduce the immunogenicity of a non-human Fv by replacing exposed residues in its framework regions that differ from those usually found in human antibodies. Because protein antigenicity may be correlated with surface accessibility, replacement of the surface residues may be sufficient to render the mouse variable domain “invisible” to the human immune system (see also Mark G. E. et al (1994) in Handbook of Experimental Pharmacology vol. 113: The pharmacology of monoclonal Antibodies, Springer-Verlag, pp 105-134). This procedure of humanisation is referred to as “veneering” because only the surface of the antibody is altered, the supporting residues remain undisturbed.

### 1.3 Bispecific Antibodies

A bispecific antibody is an antibody having binding specificities for at least two different epitopes. Methods of making such antibodies are known in the art. Traditionally, the recombinant production of bispecific antibodies is based on the co-expression of two immunoglobulin H chain-L chain pairs, where the two H chains have different binding specificities see Millstein et al, Nature 305 537-539 (1983), WO93/08829 and Traunecker et al EMBO, 10, 1991, 3655-3659. Because of the random assortment of H and L chains, a potential mixture of ten different antibody structures are produced of which only one has the desired binding specificity. An alternative approach involves fusing the variable domains with the desired binding specificities to heavy chain constant region comprising at least part of the hinge region, CH2 and CH3 regions. In one embodiment the CH1 region containing the site necessary for light chain binding is present in at least one of the fusions. DNA encoding these fusions, and if desired the L chain are inserted into separate expression vectors and are then co-transfected into a suitable host organism. It is possible though to insert the coding sequences for two or all three chains into one expression vector. In one approach, the bispecific antibody is composed of a H chain with a first binding specificity in one arm and a H-L chain pair, providing a second binding specificity in the other arm, see WO94/04690. Also see Suresh et al Methods in Enzymology 121, 210, 1986.

In one embodiment of the invention there is provided a bispecific antibody wherein at least one binding specificity of said antibody is for hIGF-1R, and said antibody neutralises the activity of hIGF-1R. Such antibodies may further comprise a human constant region of the IgG isotype, e.g. IgG1, IgG2, IgG3 or IgG4. Antibodies of the present invention may also be multispecific, for example multispecific antibodies formed by assembly of a number of antigen-binding fragments.

### 1.4 Antigen Binding Fragments

Such antigen binding fragments comprise a partial heavy or light chain variable sequence (e.g., minor deletions at the amino or carboxy terminus of the immunoglobulin variable domain) which retains the same antigen binding specificity and the same or similar neutralizing ability as the antibody from which the fragment was derived.

In certain embodiments of the invention there is provided antigen binding fragments which neutralise the activity of hIGF-1R. Such fragments may be functional antigen binding fragments of intact and/or humanised and/or chimaeric antibodies such as Fab, Fab′, F(ab′)₂, Fv, ScFv fragments of the antibodies described supra. Traditionally such fragments are produced by the proteolytic digestion of intact antibodies by e.g. papain digestion (see for example, WO 94/29348) but may be produced directly from recombinantly transformed host cells. For the production of ScFv, see Bird et al; (1988) Science, 242, 423-426. In addition, antigen binding fragments may be produced using a variety of engineering techniques as described below.

Fv fragments appear to have lower interaction energy of their two chains than Fab fragments. To stabilise the association of the V_{H} and V_{L} domains, they have been linked with peptides (Bird et al, (1988) Science 242, 423-426, Huston et al, PNAS, 85, 5879-5883), disulphide bridges (Glockshuber et al, (1990) Biochemistry, 29, 1362-1367) and “knob in hole” mutations (Zhu et al (1997), Protein Sci., 6, 781-788). ScFv fragments can be produced by methods well known to those skilled in the art see Whitlow et al (1991) Methods companion Methods Enzymol, 2, 97-105 and Huston et al (1993) Int. Rev. Immunol 10, 195-217. ScFv may be produced in bacterial cells such as E. Coli but are more preferably produced in eukaryotic cells. One disadvantage of ScFv is the monovalency of the product, which precludes an increased avidity due to polyvalent binding, and their short half-life. Attempts to overcome these problems include bivalent (ScFv′)₂ produced from ScFV containing an additional C terminal cysteine by chemical coupling (Adams et al (1993) Can. Res 53, 4026-4034 and McCartney et al (1995) Protein Eng. 8, 301-314) or by spontaneous site-specific dimerization of ScFv containing an unpaired C terminal cysteine residue (see Kipriyanov et al (1995) Cell. Biophys 26, 187-204). Alternatively, ScFv can be forced to form multimers by shortening the peptide linker to 3 to 12 residues to form “diabodies”, see Holliger et al PNAS (1993), 90, 6444-6448. Reducing the linker still further can result in ScFV trimers (“triabodies”, see Kortt et al (1997) Protein Eng, 10, 423-433) and tetramers (“tetrabodies”, see Le Gall et al (1999) FEBS Lett, 453, 164-168). Construction of bivalent ScFV molecules can also be achieved by genetic fusion with protein dimerizing motifs to form “miniantibodies” (see Pack et al (1992) Biochemistry 31, 1579-1584) and “minibodies” (see Hu et al (1996), Cancer Res. 56, 3055-3061). ScFv-Sc-Fv tandems ((ScFV)2) may also be produced by linking two ScFv units by a third peptide linker, see Kurucz et al (1995) J. Immol. 154, 4576-4582. Bispecific diabodies can be produced through the noncovalent association of two single chain fusion products consisting of V_{H} domain from one antibody connected by a short linker to the V_{L} domain of another antibody, see Kipriyanov et al (1998), Int. J. Can 77, 763-772. The stability of such bispecific diabodies can be enhanced by the introduction of disulphide bridges or “knob in hole” mutations as described supra or by the formation of single chain diabodies (ScDb) wherein two hybrid ScFv fragments are connected through a peptide linker see Kontermann et al (1999) J. Immunol. Methods 226 179-188. Tetravalent bispecific molecules are available by e.g. fusing a ScFv fragment to the CH3 domain of an IgG molecule or to a Fab fragment through the hinge region see Coloma et al (1997) Nature Biotechnol. 15, 159-163. Alternatively, tetravalent bispecific molecules have been created by the fusion of bispecific single chain diabodies (see Alt et al, (1999) FEBS Lett 454, 90-94. Smaller tetravalent bispecific molecules can also be formed by the dimerization of either ScFv-ScFv tandems with a linker containing a helix-loop-helix motif (DiBi miniantibodies, see Muller et al (1998) FEBS Lett 432, 45-49) or a single chain molecule comprising four antibody variable domains (V_{H} and V_{L}) in an orientation preventing intramolecular pairing (tandem diabody, see Kipriyanov et al, (1999) J. Mol. Biol. 293, 41-56). Bispecific F(ab′)₂ fragments can be created by chemical coupling of Fab′ fragments or by heterodimerization through leucine zippers (see Shalaby et al, (1992) J. Exp. Med. 175, 217-225 and Kostelny et al (1992), J. Immunol. 148, 1547-1553). Also available are isolated V_{H} and V_{L} domains (Domantis plc), see U.S. Pat. No. 6,248,516; U.S. Pat. No. 6,291,158; U.S. Pat. No. 6,172,197.

In one embodiment there is provided an antigen binding fragment (e.g. ScFv, Fab, Fab′, F(ab′)₂) or an engineered antigen binding fragment as described supra that specifically binds hIGF-1R neutralises the activity of hIGF-1R. The antigen binding fragment may comprise one or more of the following sequences CDRH3 as set out in SEQ. ID. NO: 1, CDRH2 as set out in SEQ. ID. NO: 2, CDRH1 as set out in SEQ. ID. NO: 3, CDRL1 as set out in SEQ. ID. NO: 4, CDRL2 as set out in SEQ. ID. NO: 5, and CDRL3 as set out in SEQ. ID. NO: 6.

### 1.5 Heteroconjugate Antibodies

Heteroconjugate antibodies also form an embodiment of the present invention. Heteroconjugate antibodies are composed of two covalently joined antibodies formed using any convenient cross-linking methods. See, for example, U.S. Pat. No. 4,676,980.

### 1.6 Other Modifications.

The interaction between the constant region of an antibody and various Fc receptors (FcγR) is believed to mediate the effector functions of the antibody which include antibody-dependent cellular cytotoxicity (ADCC), fixation of complement, phagocytosis and half-life/clearance of the antibody. Various modifications to the constant region of antibodies of the invention may be carried out depending on the desired property. For example, specific mutations in the constant region to render an otherwise lytic antibody, non-lytic is detailed in EP 0629 240B1 and EP 0307 434B2 or one may incorporate a salvage receptor binding epitope into the antibody to increase serum half life see U.S. Pat. No. 5,739,277. There are five currently recognised human Fcγ receptors, FcγR (I), FcγRIIa, FcγRIIb, FcγRIIIa and neonatal FcRn. Shields et al, (2001) J. Biol. Chem. 276, 6591-6604 demonstrated that a common set of IgG1 residues is involved in binding all FcγRs, while FcγRII and FcγRIII utilize distinct sites outside of this common set. One group of IgG1 residues reduced binding to all FcγRs when altered to alanine: Pro-238, Asp-265, Asp-270, Asn-297 and Pro-239. All are in the IgG CH2 domain and clustered near the hinge joining CH1 and CH2. While FcγRI utilizes only the common set of IgG1 residues for binding, FcγRII and FcγRIII interact with distinct residues in addition to the common set. Alteration of some residues reduced binding only to FcγRII (e.g. Arg-292) or FcγRIII (e.g. Glu-293). Some variants showed improved binding to FcγRII or FcγRIII but did not affect binding to the other receptor (e.g. Ser-267Ala improved binding to FcγRII but binding to FcγRIII was unaffected). Other variants exhibited improved binding to FcγRII or FcγRIII with reduction in binding to the other receptor (e.g. Ser-298Ala improved binding to FcγRIII and reduced binding to FcγRII). For FcγRIIIa, the best binding IgG1 variants had combined alanine substitutions at Ser-298, Glu-333 and Lys-334. The neonatal FcRn receptor is believed to be involved in both antibody clearance and the transcytosis across tissues (see Junghans R. P (1997) Immunol. Res 16. 29-57 and Ghetie et al (2000) Annu. Rev. Immunol. 18, 739-766). Human IgG1 residues determined to interact directly with human FcRn includes Ile253, Ser254, Lys288, Thr307, Gln311, Asn434 and His435. Switches at any of these positions described in this section may enable increased serum half-life and/or altered effector properties of antibodies of the invention.

Other modifications include glycosylation variants of the antibodies of the invention. Glycosylation of antibodies at conserved positions in their constant regions is known to have a profound effect on antibody function, particularly effector functioning such as those described above, see for example, Boyd et al (1996), Mol. Immunol. 32, 1311-1318. Glycosylation variants of the antibodies or antigen binding fragments thereof of the present invention wherein one or more carbohydrate moiety is added, substituted, deleted or modified are contemplated. Introduction of an asparagine-X-serine or asparagine-X-threonine motif creates a potential site for enzymatic attachment of carbohydrate moieties and may therefore be used to manipulate the glycosylation of an antibody. In Raju et al (2001) Biochemistry 40, 8868-8876 the terminal sialyation of a TNFR-IgG immunoadhesin was increased through a process of regalactosylation and/or resialylation using beta-1,4-galactosyltransferace and/or alpha, 2,3 sialyltransferase. Increasing the terminal sialylation is believed to increase the half-life of the immunoglobulin. Antibodies, in common with most glycoproteins, are typically produced as a mixture of glycoforms. This mixture is particularly apparent when antibodies are produced in eukaryotic, particularly mammalian cells. A variety of methods have been developed to manufacture defined glycoforms, see Zhang et al Science (2004), 303, 371, Sears et al, Science, (2001) 291, 2344, Wacker et al (2002) Science, 298 1790, Davis et al (2002) Chem. Rev. 102, 579, Hang et al (2001) Acc. Chem. Res 34, 727. Thus the invention contemplates a plurality of (monoclonal) antibodies (which maybe of the IgG isotype, e.g. IgG1) as herein described comprising a defined number (e.g. 7 or less, for example 5 or less such as two or a single) glycoform(s) of said antibodies or antigen binding fragments thereof.

Further embodiments of the invention include antibodies of the invention or antigen binding fragments thereof coupled to a non-proteinaeous polymer such as polyethylene glycol (PEG), polypropylene glycol or polyoxyalkylene. Conjugation of proteins to PEG is an established technique for increasing half-life of proteins, as well as reducing antigenicity and immunogenicity of proteins. The use of PEGylation with different molecular weights and styles (linear or branched) has been investigated with intact antibodies as well as Fab′ fragments, see Koumenis I. L. et al (2000) Int. J. Pharmaceut. 198:83-95.

### 2. Production Methods

Antibodies of the invention maybe produced as a polyclonal population but are more preferably produced as a monoclonal population (that is as a substantially homogenous population of identical antibodies directed against a specific antigenic binding site). It will of course be apparent to those skilled in the art that a population implies more than one antibody entity. Antibodies of the present invention may be produced in transgenic organisms such as goats (see Pollock et al (1999), J. Immunol. Methods 231:147-157), chickens (see Morrow K J J (2000) Genet. Eng. News 20:1-55, mice (see Pollock et al) or plants (see Doran P M, (2000) Curr. Opinion Biotechnol. 11, 199-204, Ma J K-C (1998), Nat. Med. 4; 601-606, Baez J et al, BioPharm (2000) 13: 50-54, Stoger E et al; (2000) Plant Mol. Biol. 42:583-590). Antibodies may also be produced by chemical synthesis. However, antibodies of the invention are typically produced using recombinant cell culturing technology well known to those skilled in the art. A polynucleotide encoding the antibody is isolated and inserted into a replicable vector such as a plasmid for further cloning (amplification) or expression. One useful expression system is a glutamate synthetase system (such as sold by Lonza Biologics), particularly where the host cell is CHO or NS0 (see below). Polynucleotide encoding the antibody is readily isolated and sequenced using conventional procedures (e.g. oligonucleotide probes). Vectors that may be used include plasmid, virus, phage, transposons, minichromsomes of which plasmids are a typical embodiment. Generally such vectors further include a signal sequence, origin of replication, one or more marker genes, an enhancer element, a promoter and transcription termination sequences operably linked to the light and/or heavy chain polynucleotide so as to facilitate expression. Polynucleotide encoding the light and heavy chains may be inserted into separate vectors and transfected into the same host cell or, if desired both the heavy chain and light chain can be inserted into the same vector for transfection into the host cell. Thus according to one aspect of the present invention there is provided a process of constructing a vector encoding the light and/or heavy chains of an antibody or antigen binding fragment thereof of the invention, which method comprises inserting into a vector, a polynucleotide encoding either a light chain and/or heavy chain of an antibody of the invention.

It is known to those skilled in the art that synthetic genes, which encode the same protein as a naturally occurring or wild type gene, may be designed by changing the codons that are used in the gene.

These design techniques involve replacing those codons in a gene that are rarely used in mammalian genes with codons that are more frequently used for that amino acid in mammalian gene. This process, called codon optimisation, is used with the intent that the total level of protein produced by the host cell is greater when transfected with the codon-optimised gene in comparison with the level when transfected with the wild-type sequence. Several method have been published (Nakamura et. al., Nucleic Acids Research 1996,24: 214-215; WO98/34640; WO97/11086).

Codon frequencies can be derived from literature sources for the highly expressed genes of many species (see e.g. Nakamura et al. Nucleic Acids Research 1996,24 : 214-215). Codon usage tables for human (have also been published (WO2005025614).

It will be immediately apparent to those skilled in the art that due to the redundancy of the genetic code, alternative polynucleotides to those disclosed herein (particularly those codon optimised for expression in a given host cell) are also available that will encode the polypeptides of the invention.

### 3.1 Signal Sequences

Antibodies of the present invention may be produced as a fusion protein with a heterologous signal sequence having a specific cleavage site at the N terminus of the mature protein. The signal sequence should be recognised and processed by the host cell. For prokaryotic host cells, the signal sequence may be for example an alkaline phosphatase, penicillinase, or heat stable enterotoxin II leaders. For yeast secretion the signal sequences may be for example a yeast invertase leader, α factor leader or acid phosphatase leaders see e.g. WO90/13646. In mammalian cell systems, viral secretory leaders such as herpes simplex gD signal and a native immunoglobulin signal sequence may be suitable. Typically the signal sequence is ligated in reading frame to DNA encoding the antibody of the invention.

### 3.2 Origin of Replication

Origin of replications are well known in the art with pBR322 suitable for most gram-negative bacteria, 2μ plasmid for most yeast and various viral origins such as SV40, polyoma, adenovirus, VSV or BPV for most mammalian cells. Generally the origin of replication component is not needed for mammalian expression vectors but the SV40 may be used since it contains the early promoter.

### 3.3 Selection Marker

Typical selection genes encode proteins that (a) confer resistance to antibiotics or other toxins e.g. ampicillin, neomycin, methotrexate or tetracycline or (b) complement auxiotrophic deficiencies or supply nutrients not available in the complex media. The selection scheme may involve arresting growth of the host cell. Cells, which have been successfully transformed with the genes encoding the antibody of the present invention, survive due to e.g. drug resistance conferred by the selection marker. Another example is the so-called DHFR selection marker wherein transformants are cultured in the presence of methotrexate. In typical embodiments, cells are cultured in the presence of increasing amounts of methotrexate to amplify the copy number of the exogenous gene of interest. CHO cells are a particularly useful cell line for the DHFR selection. A further example is the glutamate synthetase expression system (Lonza Biologics). A suitable selection gene for use in yeast is the trp1 gene, see Stinchcomb et al Nature 282, 38, 1979.

### 3.4 Promoters

Suitable promoters for expressing antibodies of the invention are operably linked to DNA/polynucleotide encoding the antibody. Promoters for prokaryotic hosts include phoA promoter, Beta-lactamase and lactose promoter systems, alkaline phosphatase, tryptophan and hybrid promoters such as Tac. Promoters suitable for expression in yeast cells include 3-phosphoglycerate kinase or other glycolytic enzymes e.g. enolase, glyceralderhyde 3 phosphate dehydrogenase, hexokinase, pyruvate decarboxylase, phosphofructokinase, glucose 6 phosphate isomerase, 3-phosphoglycerate mutase and glucokinase. Inducible yeast promoters include alcohol dehydrogenase 2, isocytochrome C, acid phosphatase, metallothionein and enzymes responsible for nitrogen metabolism or maltose/galactose utilization. Promoters for expression in mammalian cell systems include viral promoters such as polyoma, fowlpox and adenoviruses (e.g. adenovirus 2), bovine papilloma virus, avian sarcoma virus, cytomegalovirus (in particular the immediate early gene promoter), retrovirus, hepatitis B virus, actin, rous sarcoma virus (RSV) promoter and the early or late Simian virus 40. Of course the choice of promoter is based upon suitable compatibility with the host cell used for expression. In one embodiment therefore there is provided a first plasmid comprising a RSV and/or SV40 and/or CMV promoter, DNA encoding light chain variable domain (V_{L}) of the invention, κC region together with neomycin and ampicillin resistance selection markers and a second plasmid comprising a RSV or SV40 promoter, DNA encoding the heavy chain variable domain (V_{H}) of the invention, DNA encoding the γ1 constant region, DHFR and ampicillin resistance markers

### 3.5 Enhancer Element

Where appropriate, e.g. for expression in higher eukaroytics, an enhancer element operably linked to the promoter element in a vector may be used. Suitable mammalian enhancer sequences include enhancer elements from globin, elastase, albumin, fetoprotein and insulin. Alternatively, one may use an enhancer element from a eukaroytic cell virus such as SV40 enhancer (at bp 100-270), cytomegalovirus early promoter enhancer, polyma enhancer, baculoviral enhancer or murine IgG2a locus (see WO04/009823). The enhancer may be located on the vector at a site upstream to the promoter.

### 3.5.5—Polyadenylation Signals

In eukaryotic systems, polyadenylation signals are operably linked to DNA/polynucleotide encoding the antibody of this invention. Such signals are typically placed 3′ of the open reading frame. In mammalian systems, non-limiting example include signals derived from growth hormones, elongation factor-1 alpha and viral (eg SV40) genes or retroviral long terminal repeats. In yeast systems non-limiting examples of polydenylation/termination signals include those derived from the phosphoglycerate kinase (PGK) and the alcohol dehydrogenase 1 (ADH) genes. In prokaryotic system polyadenylation signals are typically not required and it is instead usual to employ shorter and more defined terminator sequences. Of course the choice of polyadenylation/termination sequences is based upon suitable compatibility with the host cell used for expression.

### 3.6 Host Cells

Suitable host cells for cloning or expressing vectors encoding antibodies of the invention are prokaroytic, yeast or higher eukaryotic cells. Suitable prokaryotic cells include eubacteria e.g. enterobacteriaceae such as Escherichia e.g. E. Coli (for example ATCC 31,446; 31,537; 27,325), Enterobacter, Erwinia, Klebsiella Proteus, Salmonella e.g. Salmonella typhimurium, Serratia e.g. Serratia marcescans and Shigella as well as Bacilli such as B. subtilis and B. licheniformis (see DD 266 710), Pseudomonas such as P. aeruginosa and Streptomyces. Of the yeast host cells, Saccharomyces cerevisiae, schizosaccharomyces pombe, Kluyveromyces (e.g. ATCC 16,045; 12,424; 24178; 56,500), yarrowia (EP402, 226), Pichia Pastoris (EP183, 070, see also Peng et al J. Biotechnol. 108 (2004) 185-192), Candida, Trichoderma reesia (EP244, 234), Penicillin, Tolypocladium and Aspergillus hosts such as A. nidulans and A. niger are also contemplated.

Although Prokaryotic and yeast host cells are specifically contemplated by the invention, host cells of the present invention are higher eukaryotic cells. Suitable higher eukaryotic host cells include mammalian cells such as COS-1 (ATCC NO: CRL 1650) COS-7 (ATCC CRL 1651), human embryonic kidney line 293, baby hamster kidney cells (BHK) (ATCC CRL. 1632), BHK570 (ATCC NO: CRL 10314), 293 (ATCC NO: CRL 1573), Chinese hamster ovary cells CHO (e.g. CHO-K1, ATCC NO: CCL 61, DHFR-CHO cell line such as DG44 (see Urlaub et al, (1986) Somatic Cell Mol. Genet. 12, 555-556)), particularly those CHO cell lines adapted for suspension culture, mouse sertoli cells, monkey kidney cells, African green monkey kidney cells (ATCC CRL-1587), HELA cells, canine kidney cells (ATCC CCL 34), human lung cells (ATCC CCL 75), Hep G2 and myeloma or lymphoma cells e.g. NS0 (see U.S. Pat. No. 5,807,715), Sp2/0, Y0.

Thus in one embodiment of the invention there is provided a stably transformed host cell comprising a vector encoding a heavy chain and/or light chain of the antibody or antigen binding fragment thereof as herein described. Such host cells comprise a first vector encoding the light chain and a second vector encoding said heavy chain.

### Bacterial Fermentation

Bacterial systems are particularly suited for the expression of antigen binding fragments. Such fragments are localised intracellularly or within the periplasma. INSOluble periplasmic proteins can be extracted and refolded to form active proteins according to methods known to those skilled in the art, see Sanchez et al (1999) J. Biotechnol. 72, 13-20 and Cupit P M et al (1999) Lett Appl Microbiol, 29, 273-277.

### 3.7 Cell Culturing Methods.

Host cells transformed with vectors encoding the antibodies of the invention or antigen binding fragments thereof may be cultured by any method known to those skilled in the art. Host cells may be cultured in spinner flasks, roller bottles or hollow fibre systems but for large scale production that stirred tank reactors are used particularly for suspension cultures. Preferably the stirred tankers are adapted for aeration using e.g. spargers, baffles or low shear impellers. For bubble columns and airlift reactors direct aeration with air or oxygen bubbles maybe used. Where the host cells are cultured in a serum free culture media, the media is supplemented with a cell protective agent such as pluronic F-68 to help prevent cell damage as a result of the aeration process. Depending on the host cell characteristics, either microcarriers maybe used as growth substrates for anchorage dependent cell lines or the cells maybe adapted to suspension culture (which is typical). The culturing of host cells, particularly invertebrate host cells may utilise a variety of operational modes such as fed-batch, repeated batch processing (see Drapeau et al (1994) cytotechnology 15: 103-109), extended batch process or perfusion culture. Although recombinantly transformed mammalian host cells may be cultured in serum-containing media such as fetal calf serum (FCS), for example such host cells are cultured in synthetic serum-free media such as disclosed in Keen et al (1995) Cytotechnology 17:153-163, or commercially available media such as ProCHO-CDM or UltraCHO™ (Cambrex N.J., USA), supplemented where necessary with an energy source such as glucose and synthetic growth factors such as recombinant insulin. The serum-free culturing of host cells may require that those cells are adapted to grow in serum free conditions. One adaptation approach is to culture such host cells in serum containing media and repeatedly exchange 80% of the culture medium for the serum-free media so that the host cells learn to adapt in serum free conditions (see e.g. Scharfenberg K et al (1995) in Animal Cell technology: Developments towards the 21st century (Beuvery E. C. et al eds), pp 619-623, Kluwer Academic publishers).

Antibodies of the invention secreted into the media may be recovered and purified using a variety of techniques to provide a degree of purification suitable for the intended use. For example the use of antibodies of the invention for the treatment of human patients typically mandates at least 95% purity, more typically 98% or 99% or greater purity (compared to the crude culture medium). In the first instance, cell debris from the culture media is typically removed using centrifugation followed by a clarification step of the supernatant using e.g. microfiltration, ultrafiltration and/or depth filtration. A variety of other techniques such as dialysis and gel electrophoresis and chromatographic techniques such as hydroxyapatite (HA), affinity chromatography (optionally involving an affinity tagging system such as polyhistidine) and/or hydrophobic interaction chromatography (HIC, see U.S. Pat. No. 5,429,746) are available. In one embodiment, the antibodies of the invention, following various clarification steps, are captured using Protein A or G affinity chromatography followed by further chromatography steps such as ion exchange and/or HA chromatography, anion or cation exchange, size exclusion chromatography and ammonium sulphate precipitation. Typically, various virus removal steps are also employed (e.g. nanofiltration using e.g. a DV-20 filter). Following these various steps, a purified (preferably monoclonal) preparation comprising at least 75 mg/ml or greater e.g. 100 mg/ml or greater of the antibody of the invention or antigen binding fragment thereof is provided and therefore forms an embodiment of the invention. Suitably such preparations are substantially free of aggregated forms of antibodies of the invention.

### 4. Pharmaceutical Compositions

Purified preparations of antibodies of the invention (particularly monoclonal preparations) as described supra, may be incorporated into pharmaceutical compositions for use in the treatment of human diseases and disorders such as Rheumatoid Arthritis, Psoriasis or Cancers e.g; Acute Lymphoblastic Leukemia, Adrenocortical Carcinoma, AIDS-Related Cancers, AIDS Related Lymphoma, Anal Cancer, Childhood Cerebellar Astrocytoma, Childhood Cerebral Astrocytoma, Colorectal Cancer, Basal Cell Carcinoma, Extrahepatic Bile Duct Cancer, Bladder Cancer, Osteosarcorna/Malignant Fibrous Histiocytoma Bone Cancer, Brain Tumors (e.g., Brain Stem Glioma, Cerebellar Astrocytoma, Cerebral Astrocytoma/Malignant Glioma, Ependymoma, Medulloblastoma, Supratentorial Primitive Neuroectodermal Tumors, Visual Pathway and Hypothalamic Glioma), Breast Cancer, Bronchial Adenomas/Carcinoids, Burkitt's Lymphoma, Carcinoid Tumor, Gastrointestinal Carcinoid Tumor, Carcinoma of Unknown Primary, Primary Central Nervous System, Cerebellar Astrocytoma, Cerebral Astrocytoma/Malignant Glioma, Cervical Cancer, Childhood Cancers, Chronic Lymphocytic Leukemia, Chronic Myelogenous Leukemia, Chronic Myeloproliferative Disorders, Colon Cancer, Colorectal Cancer, Cutaneous T-Cell Lymphoma, Endometrial Cancer, Ependymoma, Esophageal Cancer, Ewing's Family of Tumors, Extracranial Germ Cell Tumor, Extragonadal Germ Cell Tumor, Extrahepatic Bile Duct Cancer, Intraocular Melanoma Eye Cancer, Retinoblastoma Eye Cancer, Gallbladder Cancer, Gastric (Stomach) Cancer, Gastrointestinal Carcinoid Tumor, Germ Cell Tumors (e.g., Extracranial, Extragonadal, and Ovarian), Gestational Trophoblastic Tumor, Glioma (e.g., Adult, Childhood Brain Stem, Childhood Cerebral Astrocytoma, Childhood Visual Pathway and Hypothalamic), Hairy Cell Leukemia, Head and Neck Cancer, Hepatocellular (Liver) Cancer, Hodgkin's Lymphoma, Hypopharyngeal Cancer, Hypothalamic and Visual Pathway Glioma, Intraocular Melanoma, Islet Cell Carcinoma (Endocrine Pancreas), Kaposi's Sarcoma, Kidney (Renal Cell) Cancer, Laryngeal Cancer, Leukemia (e.g., Acute Lymphoblastic, Acute Myeloid, Chronic Lymphocyhc, Chronic Myelogenous, and Hairy Cell), Lip and Oral Cavity Cancer, Liver Cancer, Non-Small Cell Lung Cancer, Small Cell Lung Cancer, Lymphoma (e.g., AIDS-Related, Burkitt's, Cutaneous T-cell, Hodgkin's, Non-Hodgkin's, and Primary Central Nervous System), Waldenstrom's Macroglobulinemia, Malignant Fibrous Histiocytoma of Bone/Osteosarcoma, Medulloblastoma, Melanoma, Intraocular (Eye) Melanoma, Merkel Cell Carcinoma, Mesothelioma, Metastatic Squamous Neck Cancer with Occult Primary, Multiple Endocrine Neoplasia Syndrome, Multiple Myeloma/Plasma Cell Neoplasm, Mycosis Fungoides, Myelodysplastic Syndromes, Myelodysplastic/Myeloproliferative Diseases, Myelogenous Leukemia, Chronic Myeloid Leukemia, Multiple Myeloma, Chronic Myeloproliferative Disorders, Nasal Cavity and Paranasal Sinus Cancer, Nasopharyngeal Cancer, Neuroblastoma, Oral Cancer, Oropharyngeal Cancer, Osteosarcoma/Malignant Fibrous Histiocytoma of Bone, Ovarian Cancer, Ovarian Epithelial Cancer, Ovarian Germ Cell Tumor, Ovarian Low Malignant Potential Tumor, Pancreatic Cancer, Islet Cell Pancreatic Cancer, Paranasal Sinus and Nasal Cavity Cancer, Parathyroid Cancer, Penile Cancer, Pheochromocytoma, Pineoblastoma, Pituitary Tumor, Plasma Cell Neoplasm/Multiple Myeloma, Pleuropulmonary Blastoma, Primary Central Nervous System Lymphoma, Prostate Cancer, Rectal Cancer, Renal Cell (Kidney) Cancer, Renal Pelvis and Ureter Transitional Cell Cancer, Retinoblastoma, Rhabdomyosarcoma, Salivary Gland Cancer, Soft Tissue Sarcoma, Uterine Sarcoma, Sezary Syndrome, non-Melanoma Skin Cancer, Merkel Cell Skin Carcinoma, Small Intestine Cancer, Soft Tissue Sarcoma, Squamous Cell Carcinoma, Cutaneous T-cell Lymphoma, Testicular Cancer, Thyrnoma, Thymic Carcinoma, Thyroid Cancer, Gestational Trophoblastic Tumor, Carcinoma of Unknown Primary Site, Cancer of Unknown Primary Site, Urethral Cancer, Endometrial Uterine Cancer, Uterine Sarcoma, Vaginal Cancer, Visual Pathway and Hypothalamic Glioma, Vulvar Cancer, Waldenstrom's Macroglobulinemia, and Wilms'Tumor.

Typically such compositions comprise a pharmaceutically acceptable carrier as known and called for by acceptable pharmaceutical practice, see e.g. Remingtons Pharmaceutical Sciences, 16th edition, (1980), Mack Publishing Co. Examples of such carriers include sterilised carrier such as saline, Ringers solution or dextrose solution, buffered with suitable buffers to a pH within a range of 5 to 8. Pharmaceutical compositions for injection (e.g. by intravenous, intraperitoneal, intradermal, subcutaneous, intramuscular or intraportal) or continuous infusion are suitably free of visible particulate matter and may comprise between 0.1 ng to 100 mg of antibody, for example between 5 mg and 25 mg of antibody. Methods for the preparation of such pharmaceutical compositions are well known to those skilled in the art. In one embodiment, pharmaceutical compositions comprise between 0.1 ng to 100 mg of antibodies of the invention in unit dosage form, optionally together with instructions for use. Pharmaceutical compositions of the invention may be lyophilised (freeze dried) for reconstitution prior to administration according to methods well known or apparent to those skilled in the art. Where embodiments of the invention comprise antibodies of the invention with an IgG1 isotype, a chelator of copper such as citrate (e.g. sodium citrate) or EDTA or histidine may be added to pharmaceutical composition to reduce the degree of copper-mediated degradation of antibodies of this isotype, see EP0612251.

Effective doses and treatment regimes for administering the antibody or antigen binding fragment thereof of the invention are generally determined empirically and are dependent on factors such as the age, weight and health status of the patient and disease or disorder to be treated. Such factors are within the purview of the attending physician. Guidance in selecting appropriate doses may be found in e.g. Smith et al (1977) Antibodies in human diagnosis and therapy, Raven Press, New York but will in general be between 1 mg and 1000 mg.

Conveniently, a pharmaceutical composition comprising a kit of parts of the antibody of the invention or antigen binding fragment thereof together with other medicaments with instructions for use is also contemplated by the present invention.

The invention furthermore contemplates a pharmaceutical composition comprising a therapeutically effective amount of an antibody or antigen binding fragment thereof as herein described for use in the treatment of diseases responsive to neutralisation of the interaction between IGF-I and IGF-1R or IGF-II and IGF-IR.

In accordance with the present invention there is provided a pharmaceutical composition comprising a therapeutically effective amount of a monoclonal humanised antibody which antibody comprises a V_{H} domain selected from the group consisting of: SEQ. I.D. NO:14 and a V_{L} domain selected from the group consisting of: SEQ. I.D. NO:16

In accordance with the present invention there is provided a pharmaceutical composition comprising a therapeutically effective amount of a monoclonal humanised antibody which antibody comprises a V_{H} domain selected from the group consisting of: SEQ. I.D. NO:15 and a V_{L} domain selected from the group consisting of: SEQ. I.D. NO:16

Conveniently, a pharmaceutical composition comprising a kit of parts of the antibody of the invention or antigen binding fragment thereof together with such another medicaments optionally together with instructions for use is also contemplated by the present invention.

The invention furthermore contemplates a pharmaceutical composition comprising a therapeutically effective amount of monoclonal antibody or antigen binding fragment thereof as herein described for use in the treatment of diseases responsive to neutralisation of the activity of IGF-1R.

In another embodiment of the invention a pharmaceutical composition comprising the antibody in combination with other therapeutic agents or radiation therapy, for example in combination with other classes of drug including mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones and anti-angiogenesis agents, including anti-growth factor receptor antagonists including trastuzumab (Herceptin), Erbitux (cetuximab), anti-growth factor antibodies such as bevacizumab (Avastin), antagonists of platelet-derived growth factor receptor (PDGFR), nerve growth factor (NGFR), fibroblast growth factor receptor (FGFR), small molecular tyrosine kinase inhibitors for example lapatinib, gefitinib, etc, chemotherapeutic agents including gemcitabine, irinotecan, paclitaxel, cisplatin, doxorubicin, topotecan, cyclophosphamide, melphalan, dacarbazine, daunorubicin, aminocamptothecin, etoposide, teniposide, adriamycin, 5-Fluorouracil, cytosine arabinoside (Ara-C), Thiotepa, Taxotere, Buslfan, Cytoxin, Taxol, Methotrexate, Vinblastine, Bleomycin, Ifosfamide, Mitomycin C, Mitoxantrone, Vincristine, Vinorelbine, Carboplatin, Caminomycin, Aminopterin, Dactinomycin, used in the treatment of human diseases and disorders such as Rheumatoid Arthritis, Psoriasis or Cancers such as: Acute Lymphoblastic Leukemia, Adrenocortical Carcinoma, AIDS-Related Cancers, AIDS Related Lymphoma, Anal Cancer, Childhood Cerebellar Astrocytoma, Childhood Cerebral Astrocytoma, Colorectal Cancer, Basal Cell Carcinoma, Extrahepatic Bile Duct Cancer, Bladder Cancer, Osteosarcorna/Malignant Fibrous Histiocytoma Bone Cancer, Brain Tumors (e.g., Brain Stem Glioma, Cerebellar Astrocytoma, Cerebral Astrocytoma/Malignant Glioma, Ependymoma, Medulloblastoma, Supratentorial Primitive Neuroectodermal Tumors, Visual Pathway and Hypothalamic Glioma), Breast Cancer, Bronchial Adenomas/Carcinoids, Burkitt's Lymphoma, Carcinoid Tumor, Gastrointestinal Carcinoid Tumor, Carcinoma of Unknown Primary, Primary Central Nervous System, Cerebellar Astrocytoma, Cerebral Astrocytoma/Malignant Glioma, Cervical Cancer, Childhood Cancers, Chronic Lymphocytic Leukemia, Chronic Myelogenous Leukemia, Chronic Myeloproliferative Disorders, Colon Cancer, Colorectal Cancer, Cutaneous T-Cell Lymphoma, Endometrial Cancer, Ependymoma, Esophageal Cancer, Ewing's Family of Tumors, Extracranial Germ Cell Tumor, Extragonadal Germ Cell Tumor, Extrahepatic Bile Duct Cancer, Intraocular Melanoma Eye Cancer, Retinoblastoma Eye Cancer, Gallbladder Cancer, Gastric (Stomach) Cancer, Gastrointestinal Carcinoid Tumor, Germ Cell Tumors (e.g., Extracranial, Extragonadal, and Ovarian), Gestational Trophoblastic Tumor, Glioma (e.g., Adult, Childhood Brain Stem, Childhood Cerebral Astrocytoma, Childhood Visual Pathway and Hypothalamic), Hairy Cell Leukemia, Head and Neck Cancer, Hepatocellular (Liver) Cancer, Hodgkin's Lymphoma, Hypopharyngeal Cancer, Hypothalamic and Visual Pathway Glioma, Intraocular Melanoma, Islet Cell Carcinoma (Endocrine Pancreas), Kaposi's Sarcoma, Kidney (Renal Cell) Cancer, Laryngeal Cancer, Leukemia (e.g., Acute Lymphoblastic, Acute Myeloid, Chronic Lymphocyhc, Chronic Myelogenous, and Hairy Cell), Lip and Oral Cavity Cancer, Liver Cancer, Non-Small Cell Lung Cancer, Small Cell Lung Cancer, Lymphoma (e.g., AIDS-Related, Burkitt's, Cutaneous T-cell, Hodgkin's, Non-Hodgkin's, and Primary Central Nervous System), Waldenstrom's Macroglobulinemia, Malignant Fibrous Histiocytoma of Bone/Osteosarcoma, Medulloblastoma, Melanoma, Intraocular (Eye) Melanoma, Merkel Cell Carcinoma, Mesothelioma, Metastatic Squamous Neck Cancer with Occult Primary, Multiple Endocrine Neoplasia Syndrome, Multiple Myeloma/Plasma Cell Neoplasm, Mycosis Fungoides, Myelodysplastic Syndromes, Myelodysplastic/Myeloproliferative Diseases, Myelogenous Leukemia, Chronic Myeloid Leukemia, Multiple Myeloma, Chronic Myeloproliferative Disorders, Nasal Cavity and Paranasal Sinus Cancer, Nasopharyngeal Cancer, Neuroblastoma, Oral Cancer, Oropharyngeal Cancer, Osteosarcoma/Malignant Fibrous Histiocytoma of Bone, Ovarian Cancer, Ovarian Epithelial Cancer, Ovarian Germ Cell Tumor, Ovarian Low Malignant Potential Tumor, Pancreatic Cancer, Islet Cell Pancreatic Cancer, Paranasal Sinus and Nasal Cavity Cancer, Parathyroid Cancer, Penile Cancer, Pheochromocytoma, Pineoblastoma, Pituitary Tumor, Plasma Cell Neoplasm/Multiple Myeloma, Pleuropulmonary Blastoma, Primary Central Nervous System Lymphoma, Prostate Cancer, Rectal Cancer, Renal Cell (Kidney) Cancer, Renal Pelvis and Ureter Transitional Cell Cancer, Retinoblastoma, Rhabdomyosarcoma, Salivary Gland Cancer, Soft Tissue Sarcoma, Uterine Sarcoma, Sezary Syndrome, non-Melanoma Skin Cancer, Merkel Cell Skin Carcinoma, Small Intestine Cancer, Soft Tissue Sarcoma, Squamous Cell Carcinoma, Cutaneous T-cell Lymphoma, Testicular Cancer, Thyrnoma, Thymic Carcinoma, Thyroid Cancer, Gestational Trophoblastic Tumor, Carcinoma of Unknown Primary Site, Cancer of Unknown Primary Site, Urethral Cancer, Endometrial Uterine Cancer, Uterine Sarcoma, Vaginal Cancer, Visual Pathway and Hypothalamic Glioma, Vulvar Cancer, Waldenstrom's Macroglobulinemia, and Wilms' Tumor.

The antibody or antigen binding fragments thereof of the present invention may be used in combination with one or more other therapeutically active agents or radiation for example in combination with other classes of drug including mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones and anti-angiogenesis agents, including anti-growth factor receptor antagonists including trastuzumab (Herceptin), Erbitux (cetuximab), anti-growth factor antibodies such as bevacizumab (Avastin), antagonists of platelet-derived growth factor receptor (PDGFR), nerve growth factor (NGFR), fibroblast growth factor receptor (FGFR), small molecule anti-IGF-1R agents, small molecular tyrosine kinase inhibitors including lapatinib, gefitinib, etc, chemotherapeutic agents including gemcitabine, irinotecan, paclitaxel, cisplatin, doxorubicin, topotecan, cyclophosphamide, melphalan, dacarbazine, daunorubicin, aminocamptothecin, etoposide, teniposide, adriamycin, 5-Fluorouracil, cytosine arabinoside (Ara-C), Thiotepa, Taxotere, Buslfan, Cytoxin, Taxol, Methotrexate, Vinblastine, Bleomycin, Ifosfamide, Mitomycin C, Mitoxantrone, Vincristine, Vinorelbine, Carboplatin, Caminomycin, Aminopterin, Dactinomycin

The invention thus provides, in a further embodiment, the use of such a combination in the treatment of diseases where IGF-1 receptor signalling contributes to the disease or where neutralising the activity of the receptor will be beneficial and the use of the antibody or antigen binding fragment thereof in the manufacture of a medicament for the combination therapy of disorders such as Rheumatoid Arthritis, Psoriasis or Cancers such as: Acute Lymphoblastic Leukemia, Adrenocortical Carcinoma, AIDS-Related Cancers, AIDS Related Lymphoma, Anal Cancer, Childhood Cerebellar Astrocytoma, Childhood Cerebral Astrocytoma, Colorectal Cancer, Basal Cell Carcinoma, Extrahepatic Bile Duct Cancer, Bladder Cancer, Osteosarcorna/Malignant Fibrous Histiocytoma Bone Cancer, Brain Tumors (e.g., Brain Stem Glioma, Cerebellar Astrocytoma, Cerebral Astrocytoma/Malignant Glioma, Ependymoma, Medulloblastoma, Supratentorial Primitive Neuroectodermal Tumors, Visual Pathway and Hypothalamic Glioma), Breast Cancer, Bronchial Adenomas/Carcinoids, Burkitt's Lymphoma, Carcinoid Tumor, Gastrointestinal Carcinoid Tumor, Carcinoma of Unknown Primary, Primary Central Nervous System, Cerebellar Astrocytoma, Cerebral Astrocytoma/Malignant Glioma, Cervical Cancer, Childhood Cancers, Chronic Lymphocytic Leukemia, Chronic Myelogenous Leukemia, Chronic Myeloproliferative Disorders, Colon Cancer, Colorectal Cancer, Cutaneous T-Cell Lymphoma, Endometrial Cancer, Ependymoma, Esophageal Cancer, Ewing's Family of Tumors, Extracranial Germ Cell Tumor, Extragonadal Germ Cell Tumor, Extrahepatic Bile Duct Cancer, Intraocular Melanoma Eye Cancer, Retinoblastoma Eye Cancer, Gallbladder Cancer, Gastric (Stomach) Cancer, Gastrointestinal Carcinoid Tumor, Germ Cell Tumors (e.g., Extracranial, Extragonadal, and Ovarian), Gestational Trophoblastic Tumor, Glioma (e.g., Adult, Childhood Brain Stem, Childhood Cerebral Astrocytoma, Childhood Visual Pathway and Hypothalamic), Hairy Cell Leukemia, Head and Neck Cancer, Hepatocellular (Liver) Cancer, Hodgkin's Lymphoma, Hypopharyngeal Cancer, Hypothalamic and Visual Pathway Glioma, Intraocular Melanoma, Islet Cell Carcinoma (Endocrine Pancreas), Kaposi's Sarcoma, Kidney (Renal Cell) Cancer, Laryngeal Cancer, Leukemia (e.g., Acute Lymphoblastic, Acute Myeloid, Chronic Lymphocyhc, Chronic Myelogenous, and Hairy Cell), Lip and Oral Cavity Cancer, Liver Cancer, Non-Small Cell Lung Cancer, Small Cell Lung Cancer, Lymphoma (e.g., AIDS-Related, Burkitt's, Cutaneous T-cell, Hodgkin's, Non-Hodgkin's, and Primary Central Nervous System), Waldenstrom's Macroglobulinemia, Malignant Fibrous Histiocytoma of Bone/Osteosarcoma, Medulloblastoma, Melanoma, Intraocular (Eye) Melanoma, Merkel Cell Carcinoma, Mesothelioma, Metastatic Squamous Neck Cancer with Occult Primary, Multiple Endocrine Neoplasia Syndrome, Multiple Myeloma/Plasma Cell Neoplasm, Mycosis Fungoides, Myelodysplastic Syndromes, Myelodysplastic/Myeloproliferative Diseases, Myelogenous Leukemia, Chronic Myeloid Leukemia, Multiple Myeloma, Chronic Myeloproliferative Disorders, Nasal Cavity and Paranasal Sinus Cancer, Nasopharyngeal Cancer, Neuroblastoma, Oral Cancer, Oropharyngeal Cancer, Osteosarcoma/Malignant Fibrous Histiocytoma of Bone, Ovarian Cancer, Ovarian Epithelial Cancer, Ovarian Germ Cell Tumor, Ovarian Low Malignant Potential Tumor, Pancreatic Cancer, Islet Cell Pancreatic Cancer, Paranasal Sinus and Nasal Cavity Cancer, Parathyroid Cancer, Penile Cancer, Pheochromocytoma, Pineoblastoma, Pituitary Tumor, Plasma Cell Neoplasm/Multiple Myeloma, Pleuropulmonary Blastoma, Primary Central Nervous System Lymphoma, Prostate Cancer, Rectal Cancer, Renal Cell (Kidney) Cancer, Renal Pelvis and Ureter Transitional Cell Cancer, Retinoblastoma, Rhabdomyosarcoma, Salivary Gland Cancer, Soft Tissue Sarcoma, Uterine Sarcoma, Sezary Syndrome, non-Melanoma Skin Cancer, Merkel Cell Skin Carcinoma, Small Intestine Cancer, Soft Tissue Sarcoma, Squamous Cell Carcinoma, Cutaneous T-cell Lymphoma, Testicular Cancer, Thyrnoma, Thymic Carcinoma, Thyroid Cancer, Gestational Trophoblastic Tumor, Carcinoma of Unknown Primary Site, Cancer of Unknown Primary Site, Urethral Cancer, Endometrial Uterine Cancer, Uterine Sarcoma, Vaginal Cancer, Visual Pathway and Hypothalamic Glioma, Vulvar Cancer, Waldenstrom's Macroglobulinemia, and Wilms' Tumor.

When the antibody or antigen binding fragments thereof of the present invention are used in combination with other therapeutically active agents, the components may be administered either together or separately, sequentially or simultaneously by any convenient route.

The combinations referred to above may conveniently be presented for use in the form of a pharmaceutical formulation and thus pharmaceutical formulations comprising a combination as defined above optimally together with a pharmaceutically acceptable carrier or excipient comprise a further embodiment of the invention. The individual components of such combinations may be administered either together or separately, sequentially or simultaneously in separate or combined pharmaceutical formulations.

When combined in the same formulation it will be appreciated that the two components must be stable and compatible with each other and the other components of the formulation and may be formulated for administration. When formulated separately they may be provided in any convenient formulation, conveniently in such a manner as are known for antibodies and antigen binding fragments thereof in the art.

When in combination with a second therapeutic agent active against the same disease, the dose of each component may differ from that when the antibody or antigen binding fragment thereof is used alone. Appropriate doses will be readily appreciated by those skilled in the art.

The invention thus provides, in a further embodiment, a combination comprising an antibody or antigen binding fragment thereof of the present invention together with another therapeutically active agent.

The combination referred to above may conveniently be presented for use in the form of a pharmaceutical formulation and thus pharmaceutical formulations comprising a combination as defined above together with a pharmaceutically acceptable carrier thereof represent a further embodiment of the invention.

The following examples illustrate various aspects of the invention. These examples do not limit the scope of this invention which is defined by the appended claims.

### EXAMPLES

### Example 1

### Generation of Monoclonal Antibodies

Monoclonal antibodies (mAbs) were produced by hybridoma cells generally in accordance with the method set forth in E Harlow and D Lane, Antibodies a Laboratory Manual, Cold Spring Harbor Laboratory, 1988. SJL mice were primed and boosted by intraperitoneal injection with recombinant human IGF-1R(R&D Systems, #305-GR) in RIBI adjuvant. Spleens from responder animals were harvested and fused to X63Ag8653GFP1L5 myeloma cells to generate hybridomas. The hybridoma supernatant material was screened for binding to IGF-1R using the FMAT (AB18200) and BIAcore A100. The AB18200 was used to confirm binding to recombinant IGF-1R (R&D Systems-305-GR-050 and 391-GR-050) and HEK293T-expressed human IGF-1R, HEK293T expressed cynomolgus macaque IGF-1R and absence of binding to HEK293T-expressed human insulin receptor. The BIAcore A100 was used to estimate the kinetics of binding of hybridoma produced antibodies to recombinant IGF-1R(R&D Systems, #305-GR). Antibodies were captured onto the chip using a rabbit anti-mouse IgG (BR-1005-14, Biacore AB). Hybridomas of interest were monocloned using semi-solid media (methyl cellulose solution), Omnitrays and the ClonePix FL system.

### Example 2

### Scale-Up and Purification of Hybridoma Material and Monoclonal Antibodies

Hybridomas to be scaled up were grown in tissue culture to the scale of 4 confluent 225 cm² flasks. At this point the cells were harvested by centrifugation at 400g for 5 minutes. The pellet was resuspended with 100 ml serum free media (JRH610) to wash the cells. The cells were then centrifuged at 400g for 5 minutes. The supernatant was aspirated and discarded. 150 ml of fresh serum free media was used to resuspend the cell pellet. The cell suspension was then transferred into a fresh 225 cm2 flask and placed in an incubator for a period of 5 days. The supernatant was then harvested and centrifuged at 400g for 20 minutes. The supernatant was harvested and sterile filtered with a 0.2 μM filter in preparation for purification. The antibody was purified using protein A resin columns. The purified antibody was dialysed against PBS pH7.4.

### Example 3

### Construction of IGF-1R Expression Vectors

### Generation of Expression Cassette for Full Length Human IGF-1R

The human IGF-1R cDNA expression cassette was identical to Genbank X04434 except for a change at nucleotide 3510. This results in the silent change of the codon for glycine 1170 from “GGC” to “GGG”. Human IGF-1R cDNA was expressed from the pcDNA3.1(−) vector (Invitrogen). The sequence of human IGF-1R is set out in SEQ ID NO 44.

### Generation of Expression Cassette for Full Length Murine IGF-1R

The murine IGF-1R cDNA expression cassette was identical to Genbank AF056187 except for a change at nucleotide 3522. This results in the silent change of the codon for glycine 1174 from “GGT” to “GGG”. The murine IGF-1R cDNAs was expressed from pcDNA3.1D-V5-His TOPO vectors (Invitrogen). The sequence of murine IGF-1R is set out in SEQ ID NO 46.

Generation of Expression Cassette for Full Length Cynomolgus Macaque Monkey (Macaca fascicularis) IGF-1R

The novel sequence for cynomolgus macaque monkey IGF-1R was cloned by PCR from a cynomolgus macaque monkey kidney cDNA library. Primers were based on the human IGF-1R database entry, NM_{—}000875. PCR primers were designed with a Kozak motif at the 5′ end and with flanking BamHI and NotI restriction sites. The BamHI-NotI PCR product was cloned into pcDNA3.1D with the vector T7 sequences proximal to the 5′ end of the IGF-1R coding sequence. The cDNA obtained is 99.6% identical to the human sequence at the protein level (4aa differences from human). The sequence of cynomolgus macaque IGF-1R is set out in SEQ ID NO 45.

### Generation of Expression Cassette for Full Length Human Insulin Receptor (Type B)

A DNA cassette encoding human insulin receptor type B (SEQ ID NO 53) was cloned into pcDNA3.1 (Invitrogen). The coding sequence of SEQ ID NO 53 is identical to the sequence given in Genbank entry:M10051, with the exception of the following changes:

The nucleotide numbering is based on the “A” of the initiation methionine being nucleotide 1 (which corresponds to position 139 of the nucleotide sequence in M10051).

| Nucleotide** | Amino Acid | SEQ ID No 53 | M10051 |
|---|---|---|---|
| 511 | 171 | TAC (Tyr) | CAC (His) |
| 783 | 261 | GAT (Asp) | GAC (Asp) |
| 909 | 303 | CAG (Gln) | CAA (Gln) |
| 1343 | 448 | ATC (Ile) | ACC (Thr) |
| 1474 | 492 | CAG (Gln) | AAG (Lys) |
| 1638 | 546 | GAC (Asp) | GAT (Asp) |
| 1650 | 550 | GCA (Ala) | GCG (Ala) |
| 3834 | 1278 | AAC (Asn) | AAG (Lys) |

Vectors for human, murine and cynomolgus macaque monkey IGF-1Rs and human insulin receptor type B were expressed transiently in 293 HEK-T cells using standard protocols and Lipofectamine reagent (Invitrogen).

### Example 4

### Generation of and Expression of Recombinant Proteins Using BacMam

Construction of pFastBacMam Vector Backbone

pFastBac 1 (Invitrogen) was digested with SnaBI and Hpa1 to remove the polyhedrin promoter. This was ligated with a 3.1 kb NruI-Bst11071 fragment from pcDNA3 (Invitrogen) which contains the cytomegalovirus immediate early (CMV IE) promoter with a polylinker and BGH poly A site and the SV40 promoter driving expression of the G418 resistance gene. This vector will allow production of a baculovirus which expresses a gene under the control of the CMV promoter in mammalian cells. It is also possible to select for stable derivatives by putting cells under G418 selection.

### Human IGF-1R-Fc Fusion Protein

A plasmid designed to express human IGF-1R extracellular domain sequences fused to a factor Xa cleavage site and human Fc sequences from IgG1 was constructed. Sequences encoding the extracellular domain (amino acids 1-935) of the human IGF-1R cDNA were amplified by PCR and fused to a Factor Xa cleavage site and Fc sequences from human IgG1. The entire insert was then sub-cloned as a HindIII-BamHI fragment into the pFastBacMam expression vector. The sequence of human IGF-1R-Fc fusion protein is set out in SEQ ID NO 47.

Cynomolgus Macaque Monkey (Macaca fascicularis) IGF-1R-Fc Fusion Protein

A plasmid designed to express cynomolgus macaque monkey IGF-1R extracellular domain sequences fused to a factor Xa cleavage site and human Fc sequences from IgG1 was constructed. The human IGF-1R expression plasmid was modified by the removal of a 82 bp XbaI fragment of vector backbone by cutting with XbaI and re-ligating. This removes a second NotI site. The coding sequence for the extracellular domain of cynomolgus macaque monkey IGF-1R (amino acids 1-935) was amplified by PCR as a HindIII-NotI fragment and ligated into the modified human IGF-1R expression plasmid which had been cut with HindIII and Not Ito remove the human sequences. The sequence of cynomolgus macaque IGF-1R-Fc fusion protein is set out in SEQ ID NO 48.

### Expression of Recombinant Proteins Using BacMam

Plasmid vectors encoding human and cynomolgus macaque monkey IGF-1R extracellular domain sequences fused to a Factor Xa cleavage site and Fc sequences from human IgG1 were used to direct protein expression using the BacMam system. Baculoviruses were generated using the Invitrogen Bac-to-Bac system. The initial P0 stock was scaled to a one litre P1 stock using standard procedures. Protein production was initiated by the infection of 1-5 litres of HEK293-F cells in suspension culture with the required BacMam virus (typically at a MOI of 10 to 100 to 1 although this was usually optimized to maximize protein production). After 2-3 days culture the cell culture supernatant was harvested, cells were removed by centrifugation and the expressed protein was then purified from the cleared supernatant.

### Example 5

### Construction of IGF-1R Ligand Expression Plasmids

Gene sequences for the processed forms of IGF-I (amino acids 49-118, Swiss-prot P01343) and IGF-II (amino acids 25-91, Swiss-prot P01344) were codon optimised for E. coli expression. The genes prepared de novo by build up of overlapping oligonucleotides and cloned into the NdeI-BamHI sites of pET-21b (Novagen). For the production of biotinylated IGF-1R ligands, a C-terminal 15 amino acid biotinylation tag sequence (GLNDIFEAQKIEWHE, ref: Schatz 1993) SEQ ID NO:17 was included in the gene build up.

The sequences of human IGF-I ligand and IGF-II ligand are set out in SEQ ID NO 49. and SEQ ID NO 51 respectively.

### Example 6

### Expression and Purification of IGF-1R Ligands

Plasmids were transformed in E. coli BL21(DE3) cells then expression carried out using LB medium with 100 μg/ml ampicillin following induction with 1 mM IPTG at 37° C. for 16 hours, The cell pellets were harvested by centrifugation. IGF-1R ligands were isolated as insoluble inclusion bodies by resuspending cell pellets in 50 mM Tris pH8.0, 200 mM NaCl, 1 mM EDTA, 5 mM DTT, lysed by sonication and recovered in the inclusion body fraction by centrifugation. Soluble IGF-1R ligands were produced by solubilising the inclusion bodies in 50 mM Tris pH8.0, 6M Guanidine Hydrochloride, then rapidly diluting into a 100 fold excess volume of 50 mM Tris pH8.0, 1 mM oxidised glutathione, 1 mM reduced glutathione followed by mixing for 16 hours at 4° C. Soluble protein was concentrated and centrifuged to remove insoluble material then biologically active IGF-1R ligands purified by reverse-phase HPLC using a Spherisorb C6 column (Waters) with an acetonitrile gradient.

For IGF-1R ligands with biotinylation tags, biotinylation was carried out by adding 5 mM ATP, 5 mM MgCl₂, 1 mM d-biotin and 1 μM biotin ligase to the purified proteins. The mixture was incubated at room temperature for 3 hours. The biotinylated IGF-1R ligands were purified by size exclusion chromatography using a Superdex 75 column (GE Healthcare). Purified IGF-1R ligands were dialysed against PBS, quantified using BSA standards and a BioRad coomassie based protein assay then stored in aliquots at −80 C. Molecular weights of purified proteins were verified by mass spectroscopy. The sequences of human tagged IGF-I ligand and tagged IGF-II ligand are set out in SEQ ID NO 50. and SEQ ID NO 52 respectively.

### Example 7

### Sequencing of Variable Domains of Hybridomas

Total RNA was extracted from pellets of approximately 10⁶ cells for each hybridoma clone using the RNeasy kit from Qiagen (#74106). AccessQuick RT-PCR System (A1702) was used to produce cDNA of the variable heavy and light regions using degenerate primers specific for the murine leader sequences and murine IgG1/_{K} or IgG2b/_{K} constant regions. The purified RT-PCR fragments were cloned and a consensus sequence obtained for each hybridoma by sequence alignment, database searching and alignment with known immunoglobulin variable sequences listed in KABAT (Sequences of Proteins of Immunological Interest, 4th Ed., U.S. Department of Health and Human Services, National Institutes of Health (1987).

The sequences listing numbers of the variable domains of hybridomas 6E11, 9C7, 2B9, 15D9 and 5G4 and shown in the Table 1 below:

TABLE 1
SEQ I.D. NO: of the variable heavy and light regions of the hybridomas

| | SEQ I.D. NO: of | SEQ I.D. NO: |
|---|---|---|
| Hybridoma | variable heavy region | of variable light region |
| 6E11 | 8 | 9 |
| 9C7 | 18 | 19 |
| 2B9 | 10 | 11 |
| 5G4 | 20 | 21 |
| 15D9 | 22 | 23 |

### Example 8

### Construction of Chimaeric Antibodies

Chimaeric antibodies, comprising parent murine variable domains grafted onto human IgG1/κ wild type constant regions were constructed by PCR cloning. Based on the consensus sequence, primers to amplify the murine variable domains were designed, incorporating restriction sites required to facilitate cloning into mammalian expression vectors. The full length heavy and light chains of the 6E11 chimeric antibody (6E11c) are given in SEQ I.D. NO: 24 and SEQ I.D. NO: 25.

### Example 9

### Humanisation Strategy

Humanised antibodies were generated by a process of grafting CDRH1, CDRH2, CDRH3, CDRL1 and CDRL3 from the murine 6E11 antibody and CDRL2 from the murine 9C7 antibody onto a suitable human framework sequence.

The sequence of the humanised variable light domain of L0 is given below (SEQ I.D. NO: 16)

The sequences of the humanised variable heavy domains of H0 and H1 are given below (SEQ I.D. NO: 14 and SEQ I.D. NO: 15 respectively).

### Construction of Humanised Antibody Vectors

DNA fragments encoding the humanised variable heavy and variable light regions were constructed de novo using a PCR-based strategy and overlapping oligonucleotides. The PCR product was cloned into mammalian expression vectors containing the human gamma 1 constant region and the human kappa constant region respectively. This is the wild type Fc region.

Using a similar strategy, the variable heavy regions were also cloned onto a variant of the human gamma 1 constant region which contained two alanine substitutions L235A and G237A (EU index numbering). These constructs are referred to herein as IgG1m(AA). The two humanised constructs which comprised the IgG1m(AA) variant are set out as H0L0 IgG1m(AA) (SEQ ID NO 54 and SEQ ID NO 39) and H1L0 IgG1m(AA) (SEQ ID NO 56 and SEQ ID NO 39).

Unless otherwise stated all humanised constructs used in the examples herein comprise wild type human gamma 1 constant regions.

### Example 10

### Recombinant Antibody Expression in CHO Cells

Expression plasmids encoding the heavy and light chains respectively of chimeric or humanised antibodies were transiently co-transfected into CHO-K1 cells. In some instances the supernatant material was used as the test article in binding and activity assays. In other instances, the supernatant material was filter sterilised and the antibody recovered by affinity chromatography using a Protein A. Antibodies were also expressed in a stable polyclonal CHO cell system. DNA vectors encoding the heavy and light chains were co-electroporated into suspension CHO cells. Cells were passaged in shake flasks in MR1 basal selective medium at 37° C., 5% CO₂, 130-150 rpm until cell viability and cell counts improved. CHO cells were then inoculated into MR1 basal ×2 selective medium and incubated for 10 to 14 days at 34° C., 5% CO₂, 130-150 rpm. The cells were pelleted by centrifugation and the supernatant sterile filtered. Antibody was recovered by Protein A purification.

### Comparative Data Between Hybridomas and/or Chimaeric mAbs and/or Humanised Mabs

### Example 11

### Receptor Binding ELISA

0.4 μg/mL Histidine tagged recombinant human IGF-1R(R&D Systems, #305-GR-050) was captured onto an ELISA plate coated with 0.5-1 μg/mL of rabbit polyclonal antibody to 6×His (Abcam, #ab9108). Anti-IGF-1R antibodies from the test supernatants or purified material were titrated across the plate. The levels of receptor-bound was detected by treatment with an HRP-conjugated goat-anti-mouse IgG antibody (Dako, P0260) or goat anti-human Kappa Light Chains peroxidase conjugate (Sigma, A7164). The ELISA was developed using OPD peroxidase substrate (Sigma, P9187).

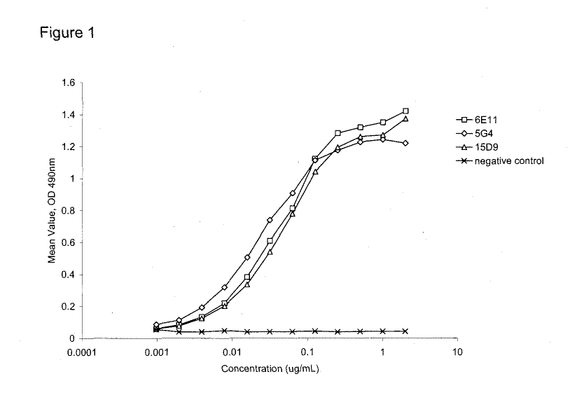

FIG. 1. shows the binding curves for murine antibodies 6E11, 5G4 and 15D9.

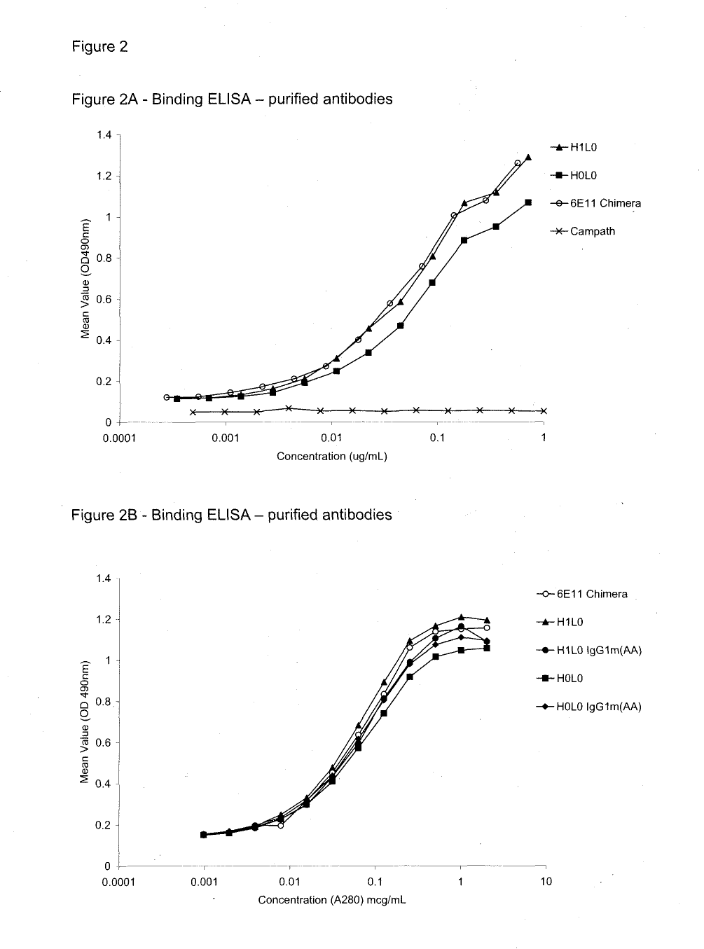

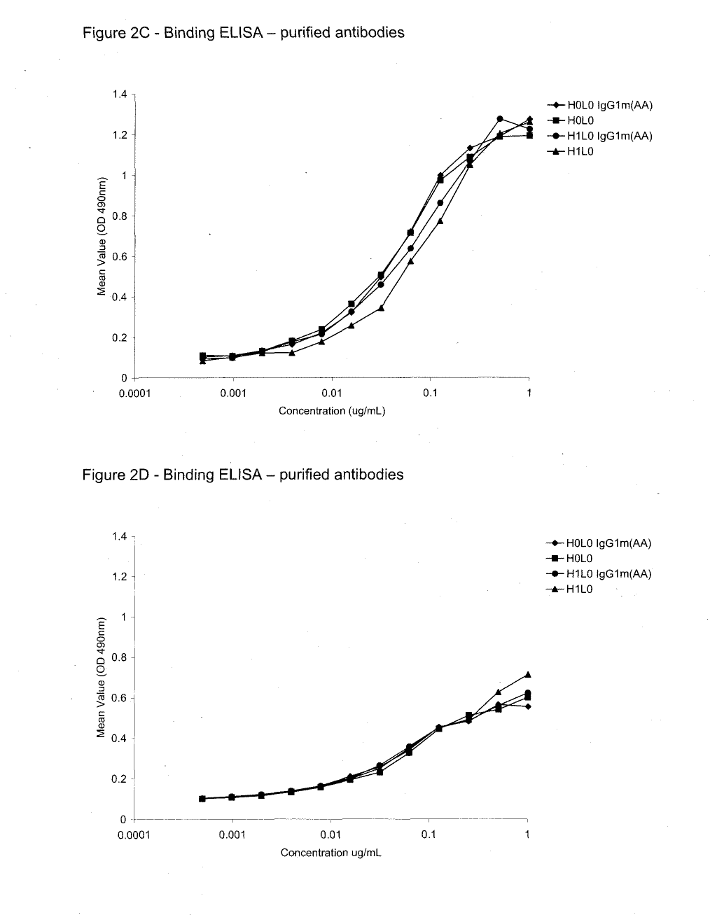

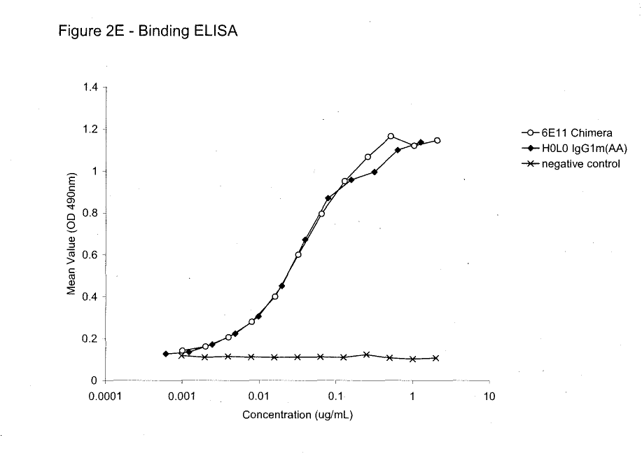

FIG. 2. shows the binding curves for H0L0 and H1L0 and H0L0 IgG1m(AA) and H1L0 IgG1m(AA) confirming they have similar binding activity when compared to the 6E11 chimaera.

### Example 12

### Receptor Down Regulation

3T3/LISN c4 cells (murine NIH 3T3 cell line expressing human IGF-1R, see Kaleko et al. (1990) Molecular and Cellular Biology, 10 (2): 464-473) were incubated with 5 μg/mL antibody at 37° C. for 24 hours then stimulated with IGF-I (100 ng/ml) for 10 mins before the cells were harvested. Lysates of these cell pellets were run on an SDS PAGE gel and transferred to PVDF membrane. IGF-1R was detected by treatment with a rabbit anti IGF-1R_{β} C-20 antibody (Santa Cruz Biotechnology, sc-713) followed by treatment with anti rabbit HRP-conjugated secondary antibody (P0217) and detected using enhanced chemiluminesence reagent (GE Healthcare).

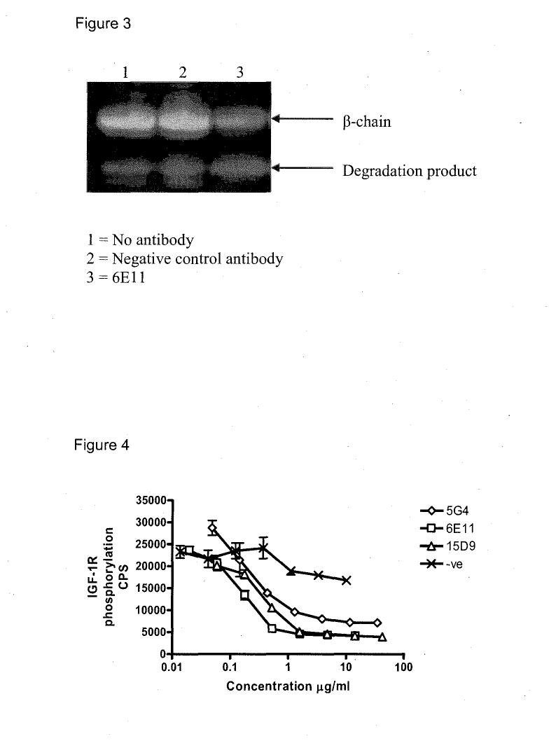

FIG. 3 shows that incubation of 3T3/LISN c4 cells with monoclonal antibody 6E11 results in down-regulation of the IGF-1R_{β} chain.

### Example 13

### Inhibition of IGF-1 Stimulated Receptor Phosphorylation

3T3/LISN c4 cells were plated at a density of 10 000 cells/well into 96 well plates and allowed to grow for 1-2 days in complete DMEM (DMEM-Hepes modification+10% FCS). Anti hIGF-1R antibodies (hybridoma supernatants or purified antibodies) were added to the cells and incubated for 1 hour. Either 30-50 ng rhIGF-1 (R&D Systems 291-G1 or 50 ng/ml rhIGF-I (see Example 5 and 6) or 100 ng/ml rhIGF-2 (R&D Systems 292-G2 or 100 ng/ml rhIGF-2 (see Example 5 and 6) was added to the treated cells and incubated for a further 20 mins to stimulate receptor phosphorylation. Cells were washed once in PBS and then lysed by the addition of RIPA lysis buffer (150 mM NaCl, 50 mM TrisHCl, 6 mM Na Deoxycholate, 1% Tween 20) plus protease inhibitor cocktail (Roche 11 697 498 001). The plate was frozen for 30 minutes or overnight. After thawing, lysate from each well was transferred to a 96 well ELISA plate pre-coated with an anti IGF-1R capture antibody (R&D Systems MAB391) at 2 μg/ml and blocked with 4% BSA/TBS. In some experiments an alternative capture antibody was used (2B9 SEQ ID NO: 10 and 11 coated at 1 μg/ml). The plate was incubated overnight at 4° C. The plate was washed with TBST (TBS+0.1% Tween 20) and a Europium labelled anti Phosphotyrosine antibody (Perkin Elmer DELFIA Eu-N1 PT66) diluted 1/2500 in 4% BSA/TBS was added to each well. After 1 hour incubation the plate was washed and DELFIA Enhancement (PerkinElmer 1244-105) solution added. After 10 min incubation the level of receptor phosphorylation was determined using a plate reader set up to measure Europium time resolved fluorescence (TRF).

FIG. 4 shows an example of the inhibition of receptor phosphorylation mediated by purified murine monoclonal antibodies 6E11, 5G4 and 15D9,

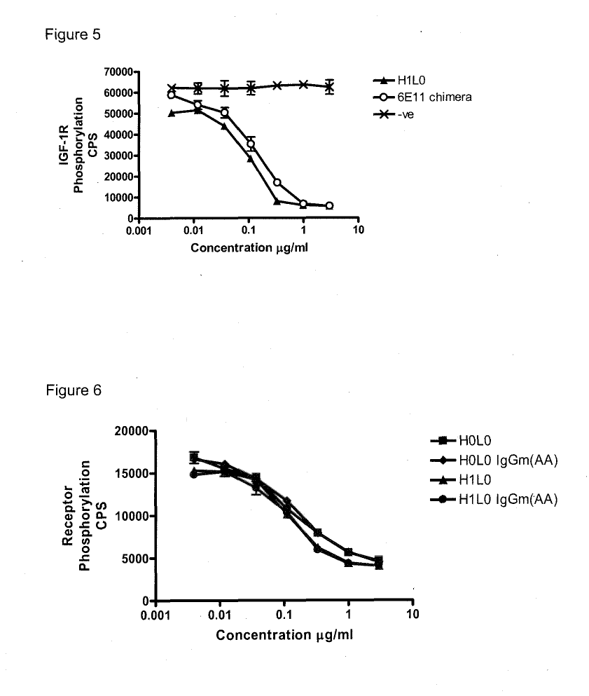

FIG. 5 shows an example of the inhibition of receptor phosphorylation mediated by H1L0 in comparison to the chimeric 6E11 antibody (6E11c).

FIG. 6 shows an example of the inhibition of receptor phosphorylation mediated by H0L0 and H1L0 in the context of a wild-type IgG1 Fc region and a substituted IgG1 Fc region (IgG1m(AA)).

### Example 14

### Competition ELISA

ELISA plates were coated with an anti human IGF-1R antagonistic antibody (MAB391, R&D Systems) at 2 μg/ml and blocked with 4% BSA/PBS. Poly-His tagged recombinant human IGF-1R(R&D Systems #305-GR) was added at 400 ng/ml in the presence of purified monoclonal antibodies (murine (6E11), chimeric or humanised) and incubated for 1 hour at room temperature. The plate was washed in TBST (TBS+0.1% Tween 20) before the addition of HRP labelled anti poly-his antibody (Sigma A7058-1VC) at 12-30 μg/ml. The plate was incubated for 1 hour before further washing and development with OPD substrate (Sigma P9187). The reaction was stopped by the addition of 2M Sulphuric acid and absorbance was measured at 490 nm.

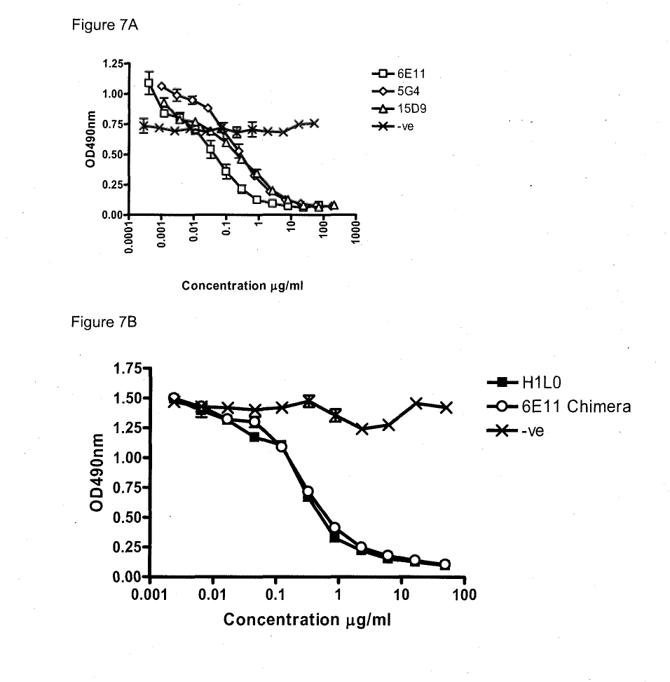

FIG. 7A shows an example of the activity of various purified murine monoclonal antibodies in the competition ELISA.

FIG. 7B shows an example of the activity of H1L0 in the competition ELISA in comparison to the 6E11 chimera (6E11c).

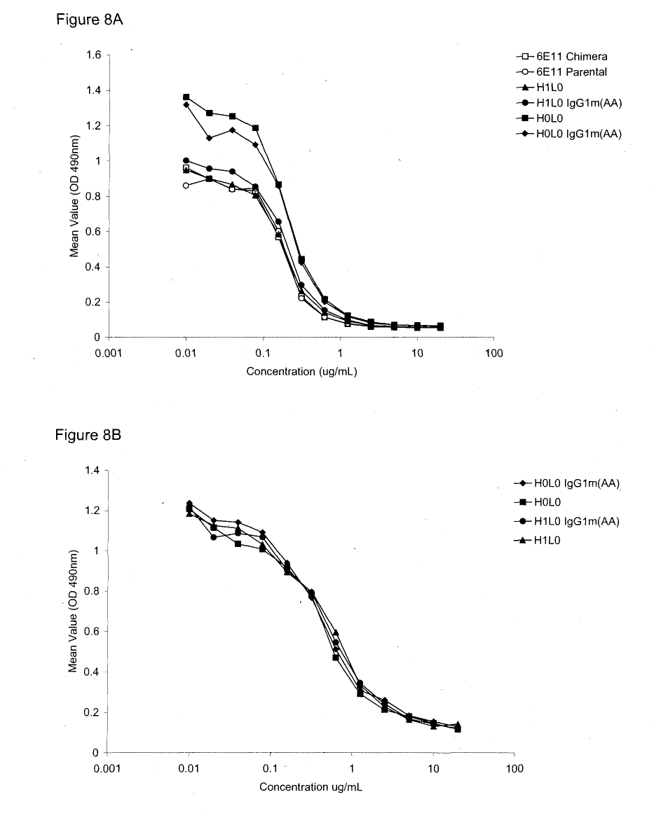

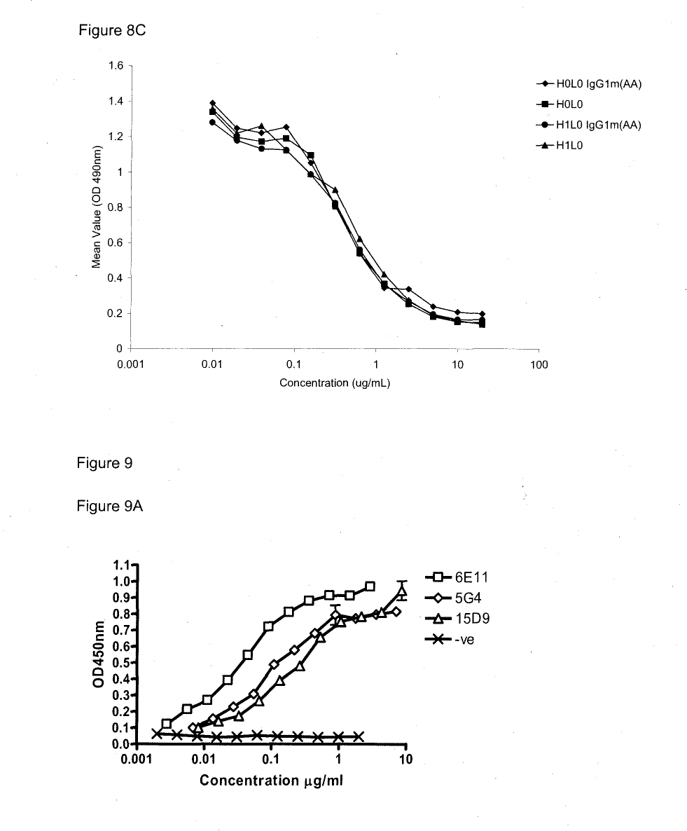

FIG. 8A shows an example of the activity of various purified humanised antibodies in the competition ELISA in comparison to the murine parental antibody (6E11) and chimera (6E11c).

FIG. 8B-C show examples of the activity of various purified humanised antibodies in the competition ELISA.

### Example 15

### Cynomolgus Macaque IGF-1R Binding ELISA

96 well ELISA plates were coated overnight with recombinant Cynomolgus macaque IGF-1R (see Example 4) at 1-2 μg/ml and blocked with 4% BSA/PBS. Purified anti-hIGF-1R antibodies were added and incubated for 1 hour at room temperature. The plates were washed in TBST and HRP conjugated anti mouse Ig (DAKO #P0260) was added to each well at 0.6-1.0 μg/ml. Plates were incubated for 1 hour at room temperature, washed with TBST and developed with OPD substrate (Sigma P9187) or TMB substrate (Sigma T8665). The reaction was stopped with 2M Sulphuric acid and the level of binding determined by measuring the absorbance at 490 nm (for OPD) and 450 nM (for TMB). For antibodies containing a human IgG1/Cκ constant region, the HRP conjugated anti mouse Ig detection antibody was substituted with a goat anti-human Kappa Light Chains peroxidase conjugate (Sigma, A7164)

FIG. 9A shows an example of purified murine monoclonal antibodies binding to recombinant cynomolgus macaque IGF-1R.

FIG. 9B shows an example of purified humanised monoclonal antibodies binding to recombinant cynomolgus macaque IGF-1R in comparison to the 6E11 chimera (6E11c).

### Example 16

### Insulin Receptor Binding ELISA

96 well ELISA plates were coated overnight with recombinant human Insulin Receptor (R&D Systems 305-GR) at 0.5 μg/ml and blocked with 4% BSA/PBS. Purified anti-hIGF-1R antibodies or mouse anti-human Insulin Receptor antibody (R&D Systems MAB15441) were added to the plates and incubated for 1 hour at room temperature before washing with TBST. HRP conjugated anti mouse Ig (DAKO #P0260) was added to each well at 1/5000 (650 ng/ml) in 4% BSA/PBS and the plates incubated for 1 hour. Plates were washed and developed by the addition of TMB substrate (Sigma T8665). The reaction was stopped with 2M Sulphuric acid and binding detected by measuring absorbance at 450 nm. For the detection of antibodies containing a human IgG1/Cκ constant region, the detection antibody listed above (HRP conjugated anti mouse Ig) was substituted with a goat anti-human Kappa Light Chains peroxidase conjugate (Sigma, A7164).

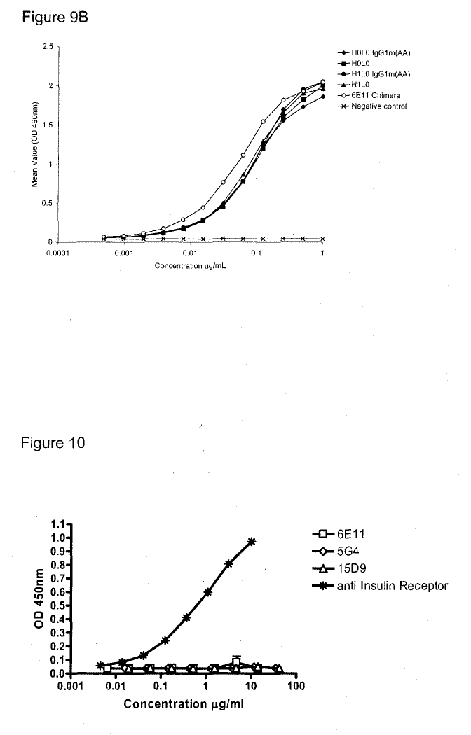

FIG. 10 shows an example of the insulin receptor binding ELISA using purified murine monoclonal antibodies. In contrast to the positive control antibody (R&D Systems MAB15441), purified antibodies 6E11, 5G4 and 15D9 showed no binding to the insulin receptor at concentrations up to 10 μg/ml.

### Example 17

### Determination of Kinetics of Binding

The binding kinetics of anti-IGF-1R antibodies for human IGF-1R were assessed using the Biacore™ system. The kinetic analysis was carried out using an antibody capture method. Briefly, an anti-mouse IgG antibody (Biacore, catalogue number BR-1005-14) was used for analysis of mouse parental antibodies and Protein A, for humanised antibodies. Either the anti mouse antibody or the Protein A was immobilised on a CM5 Biosensor chip by primary amine coupling in accordance with Biacore™ standard protocols, utilising the immobilisation Wizard facility, inherent in the machines software, (levels of 3000-4000 resonance units (RU's) where typically immobilised). Anti-IGF-1R antibodies were then captured either directly from hybridoma supernatants or from purified material. The capture levels for supernatants depended upon the starting concentration of the hybridoma and these varied between around 20 RU's to 650 RU's. For the purified material, the level captured for the antibodies tested were generally between 250 and 500 RU's. After capture, the baseline was allowed to stabilise before recombinant IGF-1R, histidine tagged material from R&D Systems (catalogue number 305-GR) was then passed over the surface at defined concentrations (usually in the range of 0-256 nM). Due to the high affinity of the interaction, dissociation times of up to one hour were used. Regeneration was by acid elution using either 100 mM phosphoric acid or 10 mM Glycine, pH 1.5, the regeneration did not significantly affect the surfaces ability to capture antibody for another analysis step. The runs were carried out at both 25° C. and 37° C. The experiments were carried out on the T100 Biacore™ system, using the T100 control and analysis software. The experimental data was fitted to the 1:1 model of binding inherent in the machines analysis software.

Tables 2-6 show a series of experiments conducted with supernatant and purified material.

TABLE 2
Kinetic data for a selection of the purified murine IGF-1R monoclonals at
25° C. and 37° C.

| | | | Affinity |
|---|---|---|---|
| | | Affinity | (nM) 25° C. |
| | Affinity (nM) 25° C. | (nM) 37° C. | (Run |
| Antibody | (Run 1-T0011 R6) | (Run 2-T0011 R4) | 3-T0022 R5) |
| 6E11 | 0.09 | 0.164 | 0.14 |
| 5G4 | 3.0 | 5.9 | Not tested |
| 15D9 | 0.233 | 0.558 | Not tested |

TABLE 3
Kinetic data for supernatant material of a H1L0 and H0L0 in comparison
with 6E11c. The run (T0037 R3) was carried out at 37° C.

| Antibodies | Ka | Kd | KD (nM) |
|---|---|---|---|
| H1L0 | 7.56e4 | 3.52e−5 | 0.47 |
| 6E11c Supernatant | 8.14e4 | 3.13e−5 | 0.38 |
| 6E11c Purified | 8.52e4 | 3.32e−5 | 0.39 |

TABLE 4
Kinetic data for supernatant material H0L0 and H0L0 IgG1m (AA) and
H1L0 and H10L0 IgG1m (AA) in comparison with 6E11c. The run
(T0040 R2) was carried out at 37° C.

| | | | |
|---|---|---|---|
| Antibodies | Ka | Kd | KD (nM) |

Run 1

| | | | |
|---|---|---|---|
| H1L0 | 7.56e4 | 3.52e−5 | 0.47 |
| ((supernatant) | | | |
| 6E11c | 8.14e4 | 3.13e−5 | 0.38 |
| (supernatant) | | | |
| 6E11c (purified) | 8.52e4 | 3.32e−5 | 0.39 |

Run 2

| | | | |
|---|---|---|---|
| H1L0 | 6.82e4 | 4.28e−5 | 0.63 |
| (supernatant) | | | |
| 6E11c (purified) | 7.59e4 | 3.25e−5 | 0.43 |

(H1L0 supernatants are the same for runs 1 and 2, however the 6E11c purified are different batches.)

TABLE 5
Kinetic data for purified H0L0 and H1L0 in comparison with the 6E11
chimera (6E11c). The run (T0041 R1) was carried out at 37° C.

| Antibodies | Ka | Kd | KD (nM) |
|---|---|---|---|
| H0L0 | 6.24e4 | 3.93e−5 | 0.63 |
| H1L0 | 6.54e4 | 2.95e−5 | 0.45 |
| 6E11c | 6.60e4 | 2.45e−5 | 0.37 |

TABLE 6
Kinetic data for purified H0L0 and H0L0 IgG1m(AA) and H1L0 and H10L0 IgG1m(AA) in
comparison with the 6E11 chimera (6E11c). Three independent runs
were carried out at 37° C.

| | Run 1-T0044 R3 | | | Run 2-T0044 R4 | | | Run 3-T0044 R6 | | |
|---|---|---|---|---|---|---|---|---|---|
| | | | KD | | | KD | | | KD |
| Antibody | Ka | Kd | (nM) | Ka | Kd | (nM) | Ka | Kd | (nM) |
| H0L0 | 5.13e4 | 2.68e5 | 0.52 | 6.62e4 | 3.97e5 | 0.59 | 6.17e4 | 5.56e5 | 0.90 |
| H0L0 | 5.40e4 | 2.67e5 | 0.49 | 7.68e4 | 4.00e5 | 0.52 | 7.38e4 | 5.17e5 | 0.77 |
| IgG1m(AA) | | | | | | | | | |
| H1L0 | 4.97e4 | 2.09e5 | 0.42 | 6.67e4 | 3.47e5 | 0.52 | 7.04e4 | 4.18e5 | 0.59 |
| H1L0 | 5.10e4 | 2.17e5 | 0.43 | 6.61e4 | 3.22e5 | 0.49 | 6.48e4 | 4.44e5 | 0.69 |
| IgG1m(AA) | | | | | | | | | |
| 6E11c | 3.99e4 | 8.71e6 | 0.22 | 6.78e4 | 2.29e5 | 0.34 | 6.75e4 | 4.02e5 | 0.59 |

### Example 18

### Inhibition of Ligand Binding Determined Using Biacore

The experiment was carried out using two different densities of captured biotinylated IGF-I. Briefly either 200 or 4000 RU's was stably captured on a streptavidin sensor chip. To test the neutralisation capacity of anti-IGF-1R antibodies, different concentrations of antibodies were pre-mixed with a fixed concentration of recombinant IGF-1R. As a control non biotinylated IGF-1 was also mixed with the same concentration of IGF-1R. This mixture was then passed over the IGF-I surface and the point of maximal association measured. This reading was then compared to a sample with the same concentration of his-tagged IGF-1R in the absence of anti-IGF-1R antibodies. The presence of a neutralising antibody blocked binding of IGF-1R to IGF-I and reduced the maximal observed association. Percentage inhibition was calculated by comparing the values. Regeneration was carried out using two pulses of 4M magnesium chloride. The experiments were carried out on a Biacore 3000 system.

Tables 7 and 8—below show the percentage inhibition obtained and also detail the concentrations of antibodies, IGF-1 and IGF-1R used to obtain these results.

TABLE 7
Inhibition Values for the 200 RU's IGF-1 Surface

| Antibody + IGF-1R complex | % Inhibition |
|---|---|
| IGF1 (125 nM) + His IGF-1R (25 nM) | 69 |
| IGF1 (500 nM) + His IGF-1R (25 nM) | 89 |
| 6E11 (125 nM) + His IGF-1R (25 nM) | 48 |
| 6E11 (500 nM) + His IGF-1R (25 nM) | 50 |

TABLE 8
Inhibition Values for the 4000 RU's IGF-1 Surface

| Antibody + IGF-1R complex | % Inhibition |
|---|---|
| IGF1 (5 μM) + His IGF-1R (50 nM) | 93 |
| IGF1 (500 nM) + His IGF-1R (50 nM) | 86 |
| 6E11 (500 nM) + His IGF-1R (50 nM) | 48 |

### Example 19

### Fluorescence Activated Cell Sorting (FACS) Analysis

Colo205 cells were stained with anti hIGF-1R purified antibodies at 10 μg/ml for 1 hour in FACs buffer (4% FCS in PBS). Cells were also stained in a suitable negative control mouse antibody (Sigma #15154). Cells were washed in FACS buffer and then stained with an anti-mouse IgG PE secondary antibody (Sigma P8547). After washing in FACS buffer and fixing in Cell Fix (Bekton Dickinson) cells were analysed by flow cytometry.

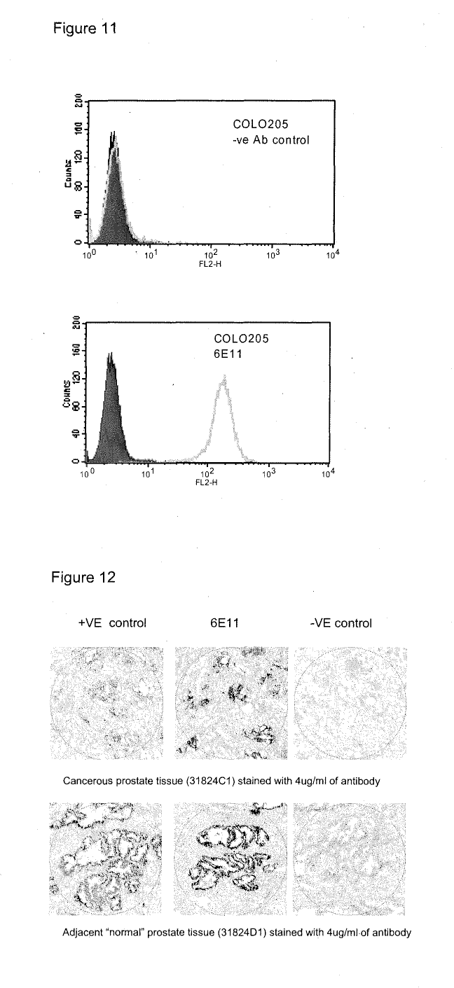

FIG. 11 demonstrates that antibody 6E11 is capable of recognising natively expressed IGF-1R on the surface of a human tumour cell line.

### Example 20

### Immunohistochemistry on Frozen Tissue Sections

Tissues were sectioned onto glass slides, fixed with acetone for 2 minutes and then loaded into an automated slide stainer (DakoCytomation S3400). Slides were then blocked and stained with murine antibodies (primary antibody) and an anti-mouse Ig-HRP secondary antibody (DakoCytomation Envision Kit) using standard immunochemical staining methods. Following this secondary incubation, the slides were washed and developed using the DakoCytomation Envision DAB solution, rinsed, dehydrated and cover-slipped for viewing. An irrelevant control antibody (mouse IgG1 purified from a MOPC21 hybridoma) was used as a negative control.

The humanised and chimeric antibodies were analysed in a similar manner except that these antibodies were biotinylated to facilitate detection. However, the presence of the biotin-tag was found to decrease the activity of these' antibodies as determined by ELISA (data not shown), therefore the concentration of primary antibody used was increased to up to 100 ug/ml. The secondary antibody listed above (DakoCytomation Envision Kit—Anti-mouse Ig-HRP conjugate) was substituted with streptavidin-HRP, (DakoCytomation Cat# 1016). An alternative irrelevant antibody was also biotinylated and used as a negative control (Sigma #15154).

The samples were analysed as follows. After calibrating the instrument using the calibration carrier (#69935000, 05041103097), the slides were loaded into the ChromaVison automated cellular imaging system and scanned at 10×. Data analysis was performed to calculate the % tissue staining (defined as brown/brown+blue*100).

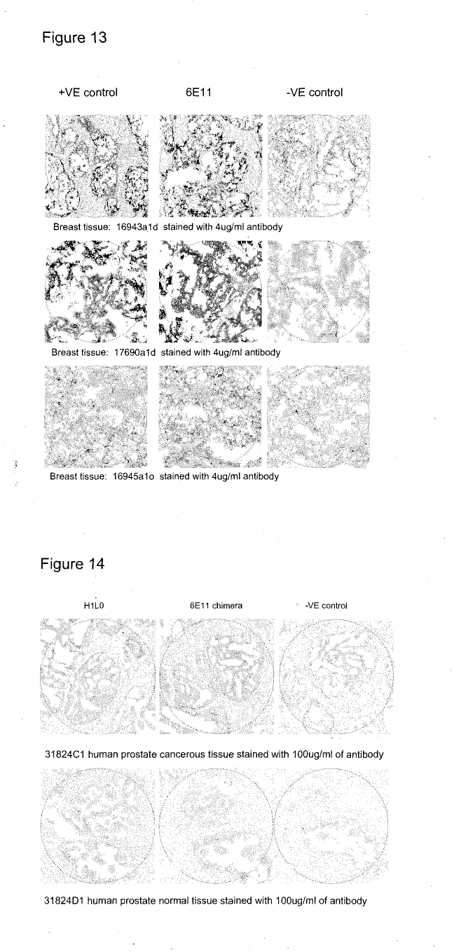

FIGS. 12 and 13 show that 6E11 stains human tumour tissue samples. A positive control antibody was included as a reference (Abcam, #4065).

FIG. 14 shows that 6E11 chimera (6E11c) and H1L0 stain human tumour tissue samples.

### Example 21

### Inhibition of AKT Signalling

Costar 96-well plates (#3598) were coated with 50 μl of 2% Gelatin in PBS and incubated in a 37° C. incubator for at least one hour. Prior to use, the plates were rinsed once with PBS. Primary human pre-adipocytes were trypsinized, centrifuge and the medium siphoned off. The cells were resuspended with 10 mL of warmed PreAdipocyte growth medium (ZenBio, #PM-1). Cell density was adjusted to 150,000 cells per mL in PreAdipocyte growth media (ZenBio). Two T225 Costar Flasks containing 50 ml of media were each seeded with 1 million cells. The remaining cells were used to seed the Gelatin-coated 96-well plates (100 μl=15,000 cells per well) using a Multidrop384 or similar instrument. The cells were incubated overnight at 37° C. in a 5% CO₂ atmosphere, 90% humidity. The following day, the medium was removed, 200 μl of Induction Medium) added and the plates covered with Breath-Easy gas-permeable film (Sigma#Z380059). The plates were incubated for 6 days at 37° C., in a 5% CO2 atmosphere, 90% humidity. After 6 days, the medium was aspirated and 200 μl of Differentiation Medium added. The plates were covered with Breath-Easy gas-permeable film and incubated for 7 days at 37° C. in a 5% CO2 atmosphere, 90% humidity. Following differentiation of the cells, the medium was aspirated and the cells rinsed once with 200 μL of PBS. 75 μl of Adipocyte Starve Medium was added and the plates covered and incubated overnight at 37° C. in a 5% CO2 atmosphere, 90% humidity. Test samples were diluted in Adipocyte Starve media at 4× the final concentration. 25 μL of diluted test compound was added to each well and incubated at 37° C. for 1 hour. IGF-I ligand (R&D Systems, #291-G1) was diluted to 30 nM in Adipocyte Starve Medium and 20 μL of 30 nM IGF-I was added to each well (final conc. 5 nM). The plates were incubated at 37° C. for precisely 5 min after which time the supernatant was removed by flicking the media into a sink. The plates were dried on paper towels.

65 μl of Complete Lysis buffer (MSD Lysis buffer containing phosphatase and protease inhibitors) was added to each well and the plate sealed with heated plate sealer. The plates were either stored at −80° C. (for later analysis) or placed on a shaker (approx. 500 rpm) for 15 mins at room temperature before performing the MSD Assay.

Levels of phosphorylated AKT (pSer473) were assessed using the MSD phosphorylation assay kit (#K111CAD). Briefly, 150 μL per well of Blocking solution (MSD Blocker A dissolved in MSD Tris Wash buffer) was added to each well of an MSD Assay plate. The plate was sealed and placed on a shaker at 300 rpm using a bench top plate shaker for 1 hour at room temperature. The Blocking solution was removed from the MSD plate(s) and the plates washed four times with 200 μL/well of 1×MSD Tris wash buffer. 50 μL/well of cell lysate from the cell plate(s) was transferred to the corresponding well of the MSD plate(s) and sealed. The plates were shaken at 300 rpm using a benchtop plate shaker for 1 hour at room temperature. The MSD plates were washed four times with 200 μL per well using 1×MSD Tris wash buffer (ELx405).

25 μL of diluted detection antibody mixture (10 nM final concentration) was added to each well of the MSD plate(s). The plates were shaken at 300 rpm using a bench top plate shaker for 1 hour at room temperature and then washed four times with 200 μL per well using 1×MSD Tris wash buffer (ELx405). 150 μL of Read Buffer T with surfactant was added to each well and the plates read with MSD 6000 SECTOR reader. Although signal intensity decreased with time in Read Buffer, the signal window typically remained steady for approximately 20-30 minutes.

Table 9 below shows a summary of the data from three independent plates and indicates that purified murine parental, chimeric and humanised Mabs inhibit IGF-I mediated induction of AKT phosphorylation. Plates 1 and 2 were run in parallel. Plate 3 was run on a separate day. The values are represented as pIC50 (=−log 10 (IC50) in g/ml)

TABLE 9
Activity of various purified antibodies in the AKT phosphorylation assay

| Antibody | Plate 1 | Plate 2 | Plate 3 |
|---|---|---|---|
| 6E11 parental | 7.75 | 7.79 | 7.67 |
| H0L0 | 7.65 | 7.76 | 7.34 |
| H1L0 | 7.62 | 7.68 | 7.30 |
| 6E11c | 7.59 | 7.32 | 7.34 |
| Negative control | 6.05 | 6.25 | <5.82 |

### Example 22

### Proliferation Assay with MCF7 Cells

MCF-7 cells (ATCC HBT-22) were seeded into 96 well plates at a density of 10000 cells/well and grown for 2 days in complete media (MEM+Earles salts+10% FCS+0.1 mg/ml bovine insulin (Sigma 10516)). Cells were washed and incubated in serum free MEM (no serum, no insulin) for 4 hours. Media was removed and replaced with a range of concentrations of purified antibodies (0.014-10 μg/ml) diluted in serum free media (100 μl/well). Cells were incubated for 1 hour before the further addition of IGF-1 (R&D Systems #291-G1) to a final concentration of 50 ng/ml. All treatments were carried out in triplicate. Cells were incubated for 5 days at 37 deg C., 5% CO2. After incubation, 15 μl of MTT dye solution (Promega #G402A) was added to each well and the plates incubated for a further 4 hours. 100 μl of Stop/Solublisation solution (Promega #G401A) was added to each well and the plate shaken gently overnight at room temperature. The following day the level of proliferation was determined by measuring the absorbance at 570 nm using a plate reader.

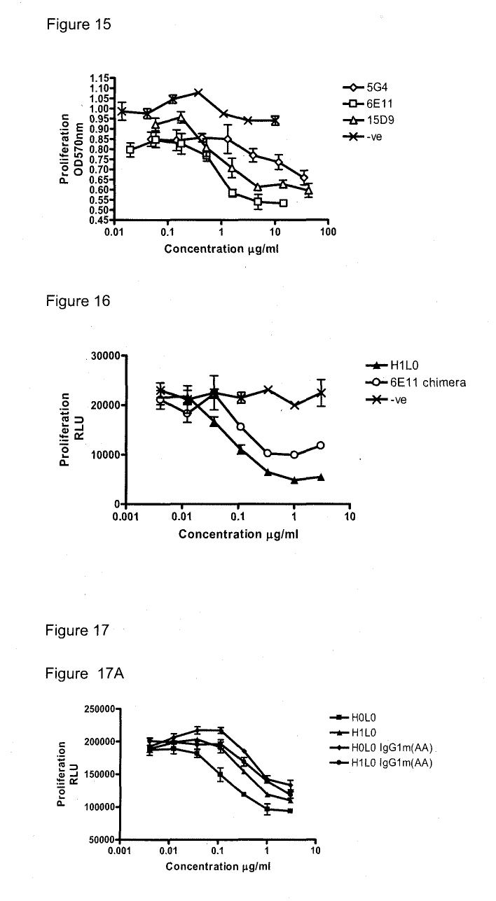

FIG. 15 shows the activity of various purified mouse monoclonal antibodies to inhibit the proliferation of tumour cells.

### Example 23

### Proliferation Assay—LISN Cells

LISN cells (3T3 hIGF-1R) were seeded into white walled 96 well plates (Corning 3610) at a density of 10 000 cells/well and grown for 1 day in complete media (DMEM-Hepes modification+10% FCS). The media was removed and cells incubated in serum free DMEM for 4 hours. Media was removed and replaced with a range of concentrations (0.0041-3 μg/ml final concentration) of purified antibodies diluted in serum free media (50 μl/well). Cells were incubated for 1 hour before the further addition of 50 μl/well IGF-1 (R&D Systems 291-G1 or IGF-I—see Examples 5 and 6) to a final concentration of 50 ng/ml. All treatments were carried out in triplicate. Cells were incubated for 3 days at 37 deg C., 5% CO2. After incubation, 100 μl of freshly prepared Promega CellTitre-Glo reagent (Promega G7571) was added to each well and the plates shaken for 2 mins. The plate was further incubated at room temperature for 10 mins to allow the signal to stabilise before measuring the luminescence signal with a Wallac Victor plate reader.

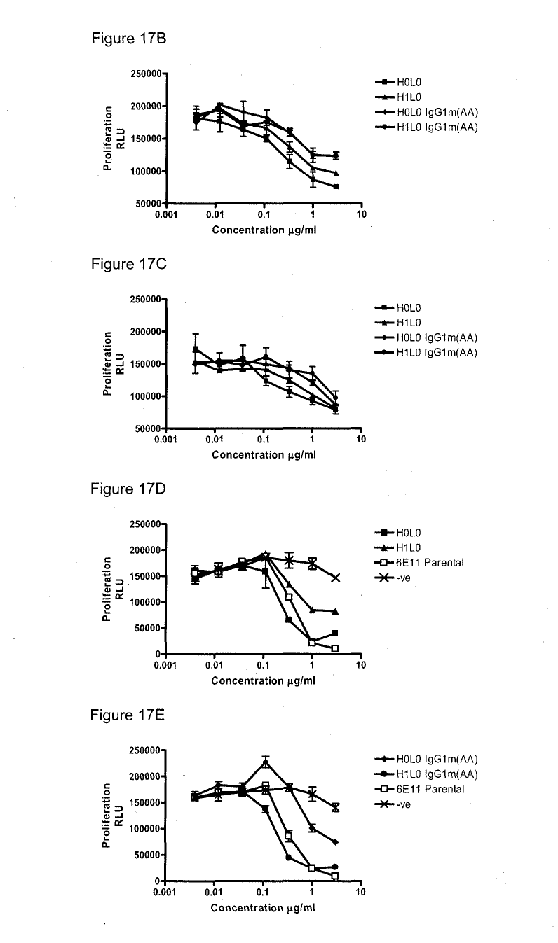

FIGS. 16 and 17 A-E show the activity of purified 6E11 murine monoclonal antibody, 6E11c H0L0 and H0L0 IgG1m(AA) and H1L0 and H1L0 IgG1m(AA). The data confirms that the H0L0 and H1L0 can inhibit tumour cell proliferation in vitro.

### Example 24

### Inhibition of Cell Cycling

NCI-H838 (ATCC CRL-5844) cells were seeded into 24 well microplates at a density of 2×10⁵ cells/well and grown overnight in 1 ml complete RPMI (RPMI+10% FCS). The following day the cells were washed with SFM (serum free RPMI media) and incubated in 1 ml of the same media for 4 hours. The media was aspirated from the cells and 500 μl of SFM containing 20 μg/ml of purified antibodies was added (10 μg/ml final concentration). Cells were incubated for 1 hour. In some wells, IGF-I (R&D Systems 291-G1) in SFM was added to a final concentration of 50 ng/ml. The treated cells were incubated overnight. The following day the cells were washed gently in PBS and then harvested by adding 200 μl of Versene solution (Invitrogen #15040). The cell suspensions were transferred to a 96 well V-bottomed plate. After pelleting the cells by centrifugation they were fixed by the addition of chilled 80% Ethanol and incubation on ice for 30 min. Cells were pelleted and re-suspended in 200 μl of 50 μg/ml Propidium Iodide, 0.1 mM EDTA, 0.1% Triton X-100, 0.05 mg/ml RNAse A. Cells were incubated on ice in the dark until being analysed by flow cytometry.

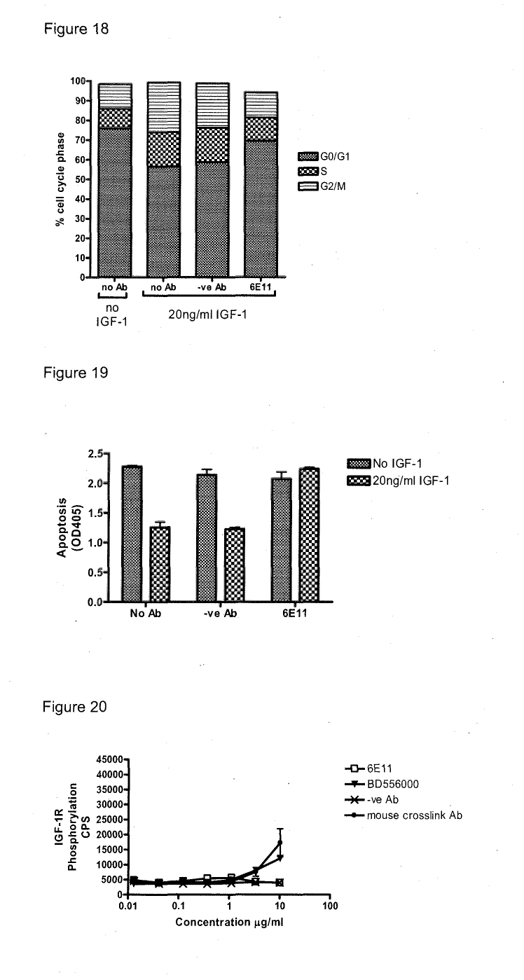

FIG. 18. shows the cell cycle status of the various treatment groups in the presence of IGF-I, the cells are induced to cycle. In the presence of 6E11 antibody, cell cycling was inhibited at levels comparable to that of cells incubated in the absence of IGF-I.

### Example 25

### Protection from Apoptosis

A 96 well microplate was seeded with NCI-H838 cells (ATCC CRL-5844) at a density of 10000 cells/well in 100 μl complete RPMI media and grown for 2 days. Cells were then washed in SFM (RPMI no serum) and incubated in 100 μl SFM for 4 hours. The media was removed prior to treatment with either no antibody, a negative control antibody or a purified anti hIGF-1R antibody at 20 μg/ml. Cells were additionally treated with either SFM alone, SFM+IGF-1 at 20 ng/ml, SFM+Camptothecin at 5 μM or SFM+Camptothecin at 5 μM+IGF-1 at 20 ng/ml. All treatments were tested in triplicate in a final volume of 100 μl. The plate was then incubated for 20 hours. The media was aspirated from the wells and the cells lysed by the addition of 200 μl of 0.5% NP-40 in PBS followed by 5 min incubation with shaking at room temperature. 20 μl of lysate was transferred to a prepared microplate from the Roche Cell Death ELISA Kit and 80 μl of incubation buffer added. The protocol described in the kit insert (Roche Cat. NO: 1 544 675) was followed and the absorbance at 405 nm measured using a microplate reader.

FIG. 19 shows that the presence of IGF-I affords NCI-H838 cells some protection from camptothecin induced apoptosis. The addition of 6E11 reversed the IGF-1 mediated protection from apoptosis

### Example 26

### Absence of Agonism in the Presence or Absence of Cross-Linking Antibodies

96 well microplates were seeded with 3T3/LISN c4 cells at a density of 10,000 cells/well in complete DMEM (DMEM Hepes modification+10% FCS) and grown for 2 days. Purified anti IGF-1R antibodies were titrated onto the cells in complete DMEM, each dilution being tested in triplicate. An antibody reported to have agonistic activity (#556000, BD Biosciences) and/or 50 ng/ml of IGF-I were included in some experiments as a positive control. Negative controls of irrelevant antibody and media alone were included. In other experiments, an anti-mouse cross-linking antibody (Sigma M8144) or an anti human cross linking antibody (Sigma 13382) were included in the antibody titration at a ratio of 2:1 [anti IGF-1 Ab]:[cross linking Ab]. Plates were incubated for 30 mins. Media was aspirated and cells were washed gently with PBS once before being lysed with RIPA lysis buffer (150 mM NaCl, 50 mM TrisHCl, 6 mM Na Deoxycholate, 1% Tween 20) plus protease inhibitor cocktail (Roche 11 697 498 001). The plate was placed at −20° C. overnight. After thawing, 100 μl samples of lysate were transferred to a 96 well ELISA plate pre-coated with an anti IGF-1R capture antibody (MAB391, R&D Systems) at 2 μg/ml and blocked with 4% BSA/TBS. The plate was incubated overnight at 4° C. The plate was washed 4 times with TBST (TBS+0.1% Tween 20) and a Europium labelled anti Phosphotyrosine antibody (DELFIA Eu-N1 PT66, PerkinElmer) diluted 1/2500 in 4% BSA/TBS was added to each well. After 1 hour incubation the plate was washed as before and DELFIA Enhancement solution (PerkinElmer 1244-105) added. After 10 min incubation the level of receptor phosphorylation was determined using a plate reader set up to measure Europium time resolved fluorescence (TRF).

FIG. 20. shows that 6E11 had no agonistic activity at concentrations up 10 μg/ml in the presence of cross-linking antibodies.

### Example 27

### Allograft Model—3T3/LISN c4

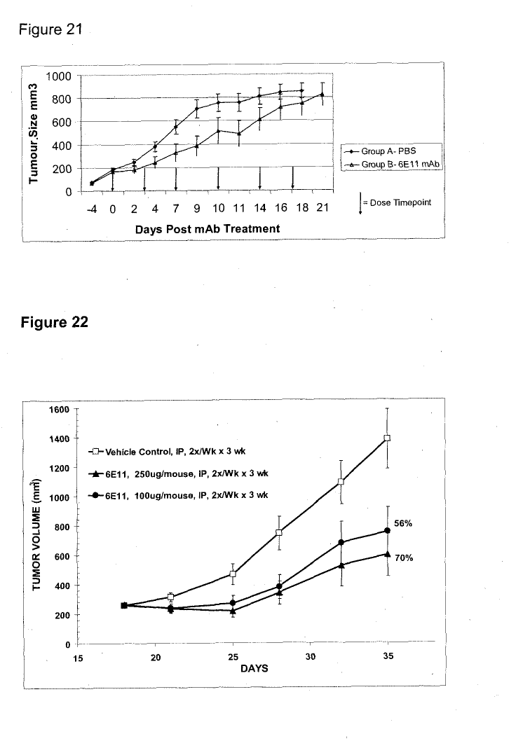

An in vivo tumour model using 3T3/LISN c4 cells was used to establish the ability of 6E11 murine monoclonal antibody to inhibit the growth of pre-established tumours in athymic nude mice. Tumours were induced by similar methods to those published in Cohen et al, Clinical Cancer Research 11:2063-2073 ((2005). In summary, 2.5×10⁶ LISN cells suspended in 0.1 ml of Matrigel™ were subcutaneously inoculated into 4-6 week old athymic CD1 nu/nu mice. Once tumours had reached approximately 150 mm³ in size, mice were treated twice weekly for three weeks with 250 μg of antibody in 0.2 ml of PBS by intraperitoneal injection. Tumours were measured by Vernier callipers across two diameters three times per week and the volume calculated using the formula (length×[width]²)/2. Data were analysed as follows: Log₁₀ transformed tumour volumes were analysed using a random coefficient regression analysis. This estimates the intercept (baseline) and slope (rate of tumour growth) for each group. Compared with the PBS treated group, there was a reduction of 31% in the growth rate in the 6E11 group (FIG. 21, p=0.0007).

In a similar experiment, nude mice were implanted sub-cutaneously with 2.5×10⁶ cells in Matrigel. Eighteen days after implantation, mice with tumor volumes of 100-200 mm³ were randomized into groups of 8 animals/treatment group. Anti-IGF-1R antibody 6E11 was administered by intraperitoneal injection at 250 μg/mouse and 100 μg/mouse dose, twice weekly for 3 weeks. Control animals received saline at the same schedule. Tumor size and mouse body weight were measured twice weekly. Compared with the saline treated group, there was a reduction of 56% and 70% in the tumour volume measured at day 35 for the 100 μg/mouse and 250 μg/mouse groups respectively (FIG. 22).

### Example 28

### Growth Inhibition of Colo205 Cell Tumours by 6E11 Mouse Parental Antibody

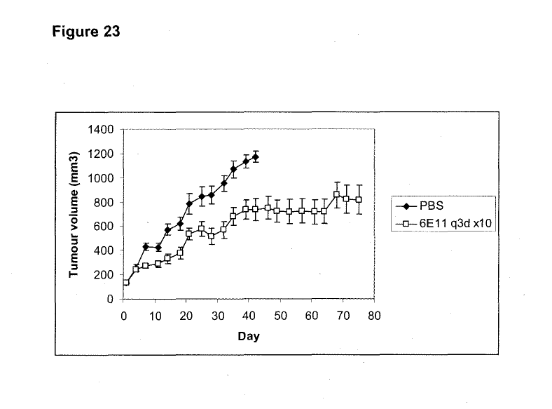

An in vivo tumour model using Colo205 cells was used to establish the ability of 6E11 murine monoclonal antibody to inhibit the growth of pre-established tumours in HRLN female nu/nu mice. 1×10⁶ Colo205 cells were suspended in 50% Matrigel and subcutaneously implanted into the flank of the nude mice. Once tumours had reached approximately 80-120 mm³ in size (equivalent to day 1 in FIG. 23), mice were treated every 3 days with 10 mg/kg of antibody by intraperitoneal injection, for a total of 10 injections. Tumours were measured by callipers and the volume calculated using the formula (length×[width]²)/2. Data were analysed as follows: Log₁₀ transformed tumour volumes were analysed using a random coefficient regression analysis. This estimates the intercept (baseline) and slope (rate of tumour growth) for each group. Compared with the vehicle control (PBS), there was a 58% reduction in the growth rate in the 6E11 antibody (FIG. 23, p=0.0019).

A similar experiment to that described above was performed on a separate occasion. However, in this second experiment using Colo205 cells no inhibition of tumour growth was observed for the 6E11 treated animals. The reasons for the absence of inhibition with 6E11 are unknown (data not shown).

A similar experiment to that described above was also performed using mice implanted with 1×10⁷ A549 cells. However, in this experiment no inhibition of tumour growth was observed for the 6E11 treated animals. The reasons for the absence of inhibition with 6E11 are unknown (data not shown).

Whilst the data from these last two experiments appears to show that the antibodies of the invention do not inhibit tumour growth in these models, it is believed that the first two tumour models (the allograft model and the first colo205 model) are more robust. A control antibody that gave a positive signal (i.e. showed inhibition of tumour growth) in these first two models showed no inhibition in the second Colo205 tumour model study or the A549 tumour model study, hence we have more confidence that the data from the first two tumour models is more indicative of activity of the test antibody than that of the second two models.

### Example 29

### Construction of Expression Vectors

Two anti-CD20 antibodies were constructed. The variable regions were obtained from the PDB protein databank (accession 2OSL). Two IgG1 constant regions were used. A wild-type IgG1 constant region and a variant Fc region which is based on a wild-type human IgG1 sequence with two substitutions (S239D/I332E, based on Kabat, EU index). The variable region and constant regions of the antibody were codon optimised and assembled by overlapping oligonucleotide PCR techniques. The variable and Fc regions were cloned using standard molecular biology techniques, into pTT5 episomal vectors and mammalian expression vectors.

The polynucleotide sequences of the anti-CD20 IgG1 heavy chain, anti-CD20 IgG1 heavy chain containing the S239D/I332E substitutions and anti-CD20 light chain Ck are given in SEQ ID: 71-73 respectively. The corresponding protein sequences are given in SEQ ID: 74-76

Anti-IGF-1R antibodies were generated using a similar approach and standard molecular biology techniques. Variable and constant regions for both heavy and light chains were assembled by overlapping oligonucleotide PCR techniques and fused together by restriction digestion and ligation into a standard mammalian expression vector.

The polynucleotide sequences of the anti-IGF-1R HO IgG1 heavy chain, anti-IGF-1R H0 IgG1 heavy chain containing the S239D/I332E substitutions and anti-IGF-1R L0 light chain Ck are given in SEQ ID: 70, 67 and 69 respectively. The corresponding protein sequences are given in SEQ ID: 37, 68 and 39 respectively.

In some cases, the polynucleotide sequence and corresponding protein sequences represent the mature antibody sequence where the signal peptide sequence has been cleaved off during post-translational processing. To direct expression of secreted proteins, it is necessary to add a signal peptide sequence to the N-terminus. One such example is given by SEQ ID: 43.

### Example 30

### Antibody Expression and Purification of Anti-CD20 and anti-IGF1R Antibodies

Adherent CHO DG44 FUT8 deleted cells were cultured until cells were in logarithmic growth phase, then rinsed and trypsinized before pelleting the cells by centrifugation. Suspension CHO-E1a cells were also cultured to logarithmic growth phase before pelleting the required cell number by centrifugation.

Both adherent and suspension CHO cells were washed in ice-cold PBS and resuspended in electroporation buffer. The cells were co-transfected by electroporation with expression plasmids containing the humanized heavy and light chain DNA sequences listed in the table below (Table 10). The electroporated cells were resuspended in a selection medium and incubated at 37° C. with 5% CO₂.

For the FUT8 deleted cells, once colonies of transfectants were observed, the cell cultures were expanded and allowed to reach confluency before harvest. For the CHO-E1 a cell lines, 10-14 day production runs were set up once viability reached 80%. Harvests were performed at the end of the production runs. Antibodies were purified from the supernatant using Hi-trap Protein-A columns.

TABLE 10
Listing of antibodies used in this study

| | | | Substitutions | |
|---|---|---|---|---|
| | | | in IgG1 | SEQ ID pair |
| | Target of | | constant | used for |
| ID | antibody | Host cell line | domain | antibody |
| CD20-A | CD20 | CHO-E1a | None | 74 + 76 |
| CD20-B | CD20 | FUT8 deleted | None | 74 + 76 |
| | | CHO DG44 | | |
| CD20-C | CD20 | CHO-E1a | S239D/I332E | 75 + 76 |
| CD20-D | CD20 | FUT8 deleted | S239D/I332E | 75 + 76 |
| | | CHO DG44 | | |
| IGF1R-E | IGF-1R | CHO-E1a | None | 37 + 39 |
| IGF1R-F | IGF-1R | FUT8 deleted | None | 37 + 39 |
| | | CHO DG44 | | |
| IGF1R-G | IGF-1R | CHO-E1a | S239D/I332E | 68 + 39 |
| IGF1R-H | IGF-1R | FUT8 deleted | S239D/I332E | 68 + 39 |
| | | CHO DG44 | | |

### Example 31

### Binding to Recombinant IGF-1R

96-well high binding plates were coated with 1 μg/ml of anti-his-tag antibody (Abcam, ab9108)) in PBS and stored overnight at 4° C. The plates were washed twice with Tris-Buffered Saline with 0.05% of Tween-20. 200 μL of blocking solution (5% BSA in DPBS buffer) was added in each well and the plates were incubated for at least 1 hour at room temperature. Another wash step was then performed. 0.4 μg/mL of recombinant human IGF-1R (R&D systems) was added to each well at 50 μL per well. The plates were incubated for an hour at room temperature and then washed. The test antibodies were successively diluted across the plate in blocking solution. After one hour incubation, the plates were washed. Goat anti-human kappa light chain specific peroxidase conjugated antibody was diluted in blocking solution to 1 μg/mL and 50 μL was added to each well. The plates were incubated for one hour. After another wash step, 50 μl of OPD SigmaFast substrate solution were added to each well and the reaction was stopped 15 minutes later by addition of 25 μL of 3M sulphuric acid. Absorbance was read at 490 nm using the VersaMax Tunable Microplate Reader (Molecular Devices) using a basic endpoint protocol.

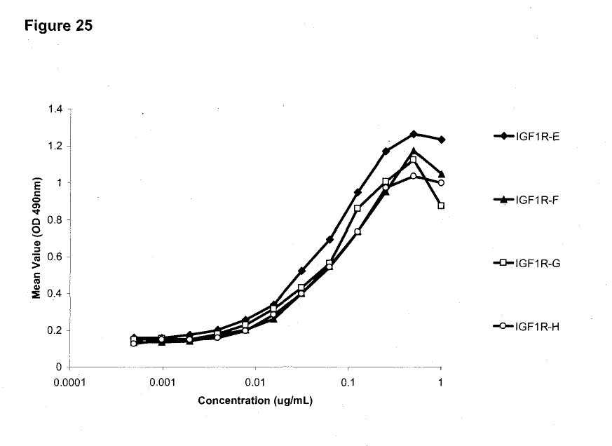

As illustrated in FIG. 25, antibody samples IGF1R-E, -F, -G and -H show comparable binding to recombinant human IGF-1R. This figure represents a composite of two separate assays, with sample IGF1R-E assessed in one assay and samples IGF1R-F, -G and -H assessed in a separate assay.

### Example 32

### Glycoprofiling of Anti IGF-1R Antibodies Expressed Using the FUT8 Deleted CHO DG44 Cell Line

Anti IGF-1R antibodies made using the FUT8 deleted CHO DG44 cell line and the CHO-E1a cell line (antibodies IGF1R-E and IGF1R-F) were reduced then de-glycosylated by digestion with PNGase-F. Liquid chromatography/mass spectrometry (LC/MS) was performed on the oligosaccharides to calculate the oligosaccharide-specific mass. The derived structures are suggestions made from interpretation of the oligosaccharide masses.

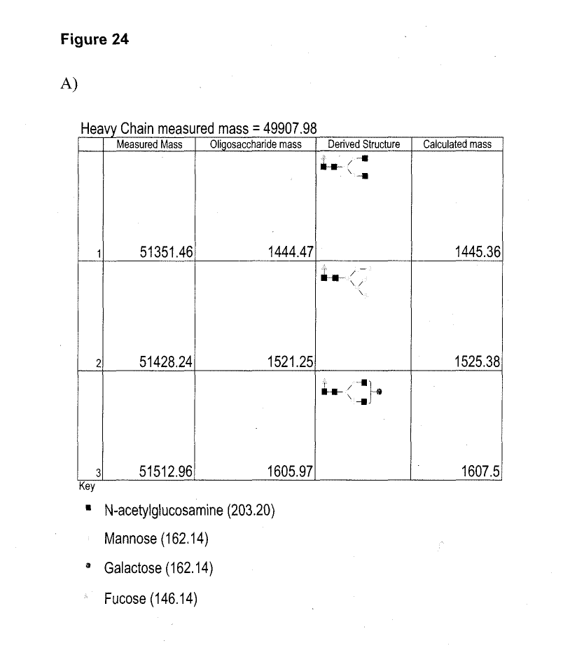

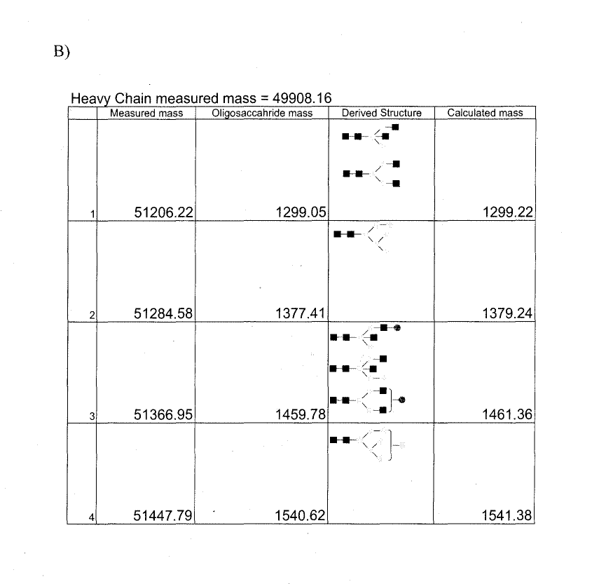

FIG. 24 summarises the oligosaccharide compositions obtained from the antibody samples IGF1R-E and IGF1R-F. It shows that all oligosaccharide species obtained from the IGF1R-F antibody sample are fucose negative.

### Example 33

### Expression/production of FcγRIIIa (V and F Variants)

The extracellular domains of the FcγRIIIa receptors were cloned with a CD33 signal sequence and a 10×His Tag into pFastBacMam-1 plasmids. SEQ ID: 77 and SEQ ID: 78 give the protein sequences of the expression cassettes for the V and F variants of FcγRIIIa respectively. 10 L scale wave-bag cultures of HEK cells were infected with Bacmam virus. Supernatant was harvested and concentrated to 1 L by tangential flow filtration (10 kmwco) and buffer exchanged into 50 mM HEPES pH 7.7, 150 mM NaCl, 50 mM Imidazole. Purification followed a two step chromatography process; Immobilized Metal Affinity Chromatography (5 ml H is Trap crude FF, GE HEalthcare) followed by Size Exclusion Chromatography on Superdex 75 (XK26/60, GE Healthcare). A HEPES buffer system was used throughout final buffer 50 mM HEPES pH 7.7, 150 mM NaCl. A single peak were observed on a size exclusion column. The final protein product gave a smeared appearance on an SDS-PAGE gel indicative of heterogeneous glycosylation.

### Example 34

### Kinetics of Binding to FcγRIIIa Receptors

Qiagen anti-polyhistidine antibody (Cat No. 34670) was immobilised on CM5 biosensor chip using standard NHS/EDC activation, the antibody was diluted to 50 ug/ml in acetate pH4.0 buffer and passed over the activated surface for 20 minutes at 5 ul/minute, the surface was then blocked with ethanolamine. Prior to use the chip was conditioned by performing several regeneration steps using 100 mM phosphoric acid.

For analysis of the interaction of the FcγRIIIa with various antibody constructs, the poly-histidine tagged receptors were captured to around 20 RU's. Antibodies were injected over the captured surface at 512, 128, 32, 8 and 2 nM with an injection of buffer over the receptor captured surface used for double referencing. Regeneration was carried out using 100 mM phosphoric acid following each antibody/buffer injection. The run was carried out using HBS-EP buffer and carried out at 25° C. on a T100 Biacore machine. The data was analysed using the analysis software inherent to the machine using the 1:1 and Bivalent models. For these experiments, generally the Bivalent model provided a better fit to the experimental data.

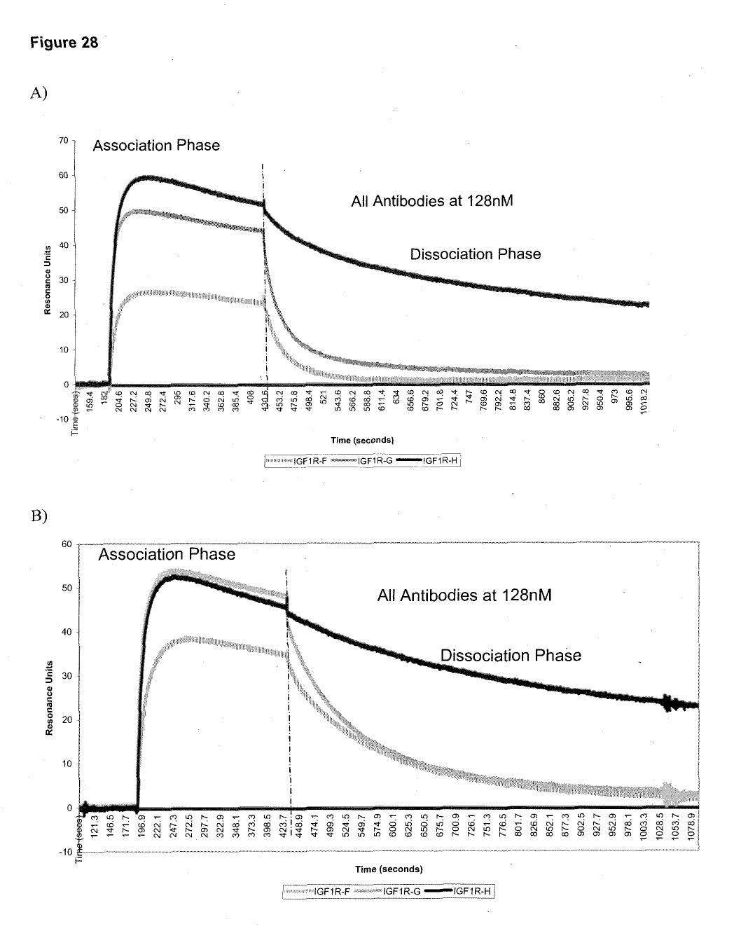

Results are shown in Table 11 and FIG. 27 (anti-CD20 antibodies) and Table 12 and FIG. 28 (anti-IGF-1R antibodies). FIGS. 27 and 28 show a representative trace at one antibody concentration for the different antibodies to illustrate the differences in the off-rate between the different constructs. The data is consistent with improved binding to FcγRIIIa for antibodies with altered Fc regions (either by substitution or glycoengineering or both). FIGS. 27 and 28 illustrate the improved off-rate with antibodies with altered Fc regions (either by substitution or glycoengineering or both) and in particular for antibodies CD20-D and IGF-1R-H.

TABLE 11
Kinetics of binding of anti-CD20 antibodies to FcγRIIIa

| Anti- | Affinity (nM) | | | |
|---|---|---|---|---|
| CD20 | FcγRIIIa | | | |
| antibody | (Phe) | FcγRIIIa (Val) | FcγRIIIa (Phe) | FcγRIIIa (Val) |
| sample | 1:1 Model | 1:1 Model | Bivalent model | Bivalent model |
| CD20-B | 20.7 | 5.9 | 75.6 | 74.1 |
| CD20-C | 5.0 | 3.7 | 34.6 | 14.2 |
| CD20-A | Off | 39.5 | 78.2 | 274 |
| | Rate | | | |
| | Too fast to | | | |
| | measure | | | |
| CD20-D | 0.449 | 0.412 | 29.8 | 1.2 |

TABLE 12
Kinetics of binding of anti-IGF-1R antibodies to FcγRIIIa

| Anti-IGF- | Affinity (nM) | | | |
|---|---|---|---|---|
| 1R | FcγRIIIa | | | |
| antibody | (Phe) | FcγRIIIa (Val) | FcγRIIIa (Phe) | FcγRIIIa (Val) |
| sample | 1:1 Model | 1:1 Model | Bivalent model | Bivalent model |
| IGF1R-F | 59.6 | 12.1 | 131.2 | 145.5 |
| IGF1R-H | 1.11 | 0.832 | 51.7 | 2.7 |
| IGF1R-G | 14.9 | 7.27 | 88.9 | 68.8 |

### Example 35

### ADCC Assays

The assay is based on the method described in J. Imm. Meth. (1995)184:29-38.

Europium labelled target cells (RAJI cells) were prepared as follows. RAJI cells were harvested and counted and prepared at final density of 10⁷ cells in a 15 ml falcon tube. Cells were washed once with HEPES buffer (50 mM HEPES, 83 mM NaCl, 5 mM KCl, 2 mM MgCl₂, pH7.4), pelleted and 1 ml of ice cold Europium labelling buffer (HEPES buffer containing 600 uM EuCl₃, 3 mM DTPA and 25 mg/l of dextran sulphate) was added. The cell suspension was flicked vigorously at the start of the labelling and then every 10 minutes during the 30 minute incubation period on ice. Following labelling, 10 ml of ice cold repair buffer (HEPES buffer containing 294 mg/l CaCl₂.2H₂O and 1.8 g/l D-Glucose, pH7.4) was added and the cells incubated on ice for a further 10 minutes. The cells were then centrifuged, the supernatant decanted and the cells washed twice further with repair buffer and then once with complete medium. The labelled cells were counted and resuspended in serum free medium at 10⁵/ml. The cells were stored on ice.

Human purified blood mononuclear cells (PBMC's or effectors) were prepared as follows. 100 ml of whole blood was centrifuged at 2000 rpm 10 mins, and the serum removed. The remaining sample was diluted to twice the original volume with PBS. The density gradient tubes were prepared by adding 15 mls lymphoprep and centrifuged for 1 min at 1500 rpm. 25 ml of blood suspension was added to the density gradient tubes and centrifuged at 2500 rpm for 20 mins with the centrifuge brake off. The top 10 ml of supernatant was discarded. The remainder (including the “buffy” layer) was poured into a clean tube, topped up with PBS and centrifuged at 1500 rpm for 5 minutes. The supernatant was discarded, the cell pellets were pooled, washed once in medium and centrifuged. The cells were counted and diluted to 5×10⁶/ml in serum free medium.

The assay plates were set up as follows using 96-well round bottom plates. The test antibodies were diluted serum free RPMI medium at 12 μg/ml (4 μg/ml final concentration) in a 1.0 ml final volume. Further 3-fold dilutions were made in serum free RPMI medium.

50 μl antibody dilution was added to the appropriate wells according to the plate layout shown below. 50 μl medium to all wells in rows A, and H. 100 μl of Europium labelled target cells were added to all wells in appropriately labelled plates. 20 μl of 10× triton was added to all wells in row H on all plates. The plates were incubated at 4° C. for minimum 30 minutes.

50 μl medium was added to all wells in columns labelled targets alone. 50 μl PBMCs was added to all wells in columns labelled effector:targets to give a final effector : target ratio of 25:1. The plates were centrifuge at 1500 rpm for 3 mins and incubate 37° C. for 3-4 hrs. 200 μl enhancement solution (Wallac/Perkin Elmer Catalogue #1244-105) was added to a 96-well Nunc immunosorbant ELISA plates (one Elisa plate for correspond with each assay plate). 20 μl of supernatant from the assay plate was transferred to the ELISA plate and incubated at room temperature on plate shaker minimum 30 minutes, or overnight at 4° C. Europium release was measured using time-delayed fluorimetry (Wallac Victor plate reader). Spontaneous lysis=measurement of Europium released from cells and medium alone. Maximum lysis=non-specific lysis of target cells by addition of Triton-X100 (non-ionic detergent).

### 96-Well Plate Layout

| | Effector: Targets | | | | Targets | | | Effector: Targets | | | | Targets | | |
|---|---|---|---|---|---|---|---|---|---|---|---|---|---|---|
| Column No. | 1 | | 2 | 3 | 4 | 5 | 6 | 7 | 8 | | 9 | 10 | 11 | 12 |
| Row A | Spontaneous | | | | | | | | | | | | | |
| | release | | | | | | | | | | | | | |
| Row B | 3 | ug/ml | | | | | | | 0.003 | ug/ml | | | | |
| Row C | 1 | ug/ml | | | | | | | 0.001 | ug/ml | | | | |
| Row D | 0.3 | ug/ml | | | | | | | 0.0003 | ug/ml | | | | |
| Row E | 0.1 | ug/ml | | | | | | | 0.0001 | ug/ml | | | | |
| Row F | 0.03 | ug/ml | | | | | | | 0.00003 | ug/ml | | | | |
| Row G | 0.01 | ug/ml | | | | | | | 0.00001 | ug/ml | | | | |

| | |
|---|---|
| Row H | Maximum release |

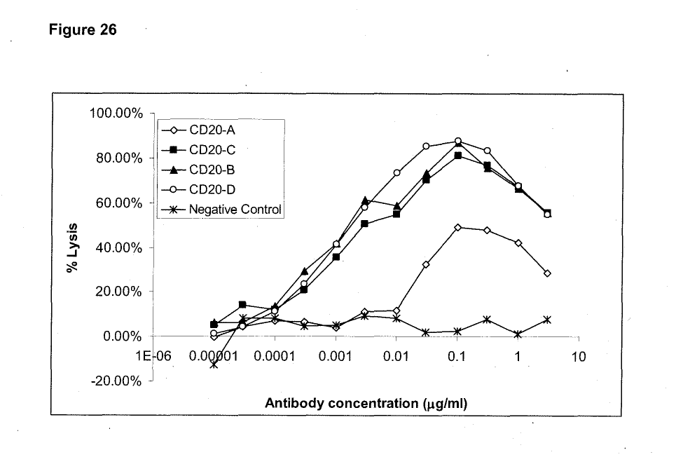

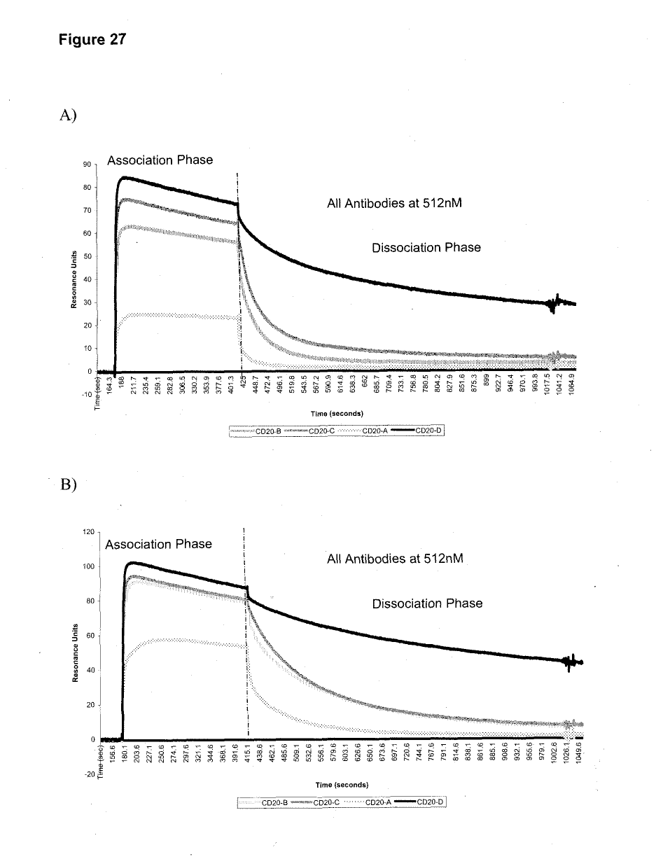

The results of one ADCC assay for the anti-CD20 antibodies are shown in FIG. 26 and confirm that antibody samples CD20-A, -B, -C and -D show specific activity against RAJI cells, with samples CD20-B, -C and -D showing comparable activity. The assay has been repeated a total of five times using PBMC effector cells from five different donors. In all cases the same trend was seen.

It would also be possible to run a similar assay using alternative target cells lines which express different levels of CD20. Examples of such cell lines include Daudi, Ramos, DOHH-2, Granta-519, FL-18. Alternatively it is possible to engineering cell lines to express different levels of CD20. An example of this methodology is given is reported by van Meerten et al. (Clinical Cancer Research Vol. 12, 4027-4035, Jul. 1, 2006). For both approaches, it will be necessary to optimise the assay for each target:effector combination by for example altering the target cell loading conditions, or the effector:target cell ratio, incubation time or by using alternative read-outs such as LDH. It is anticipated that an alternative target cell line may offer the opportunity to distinguish between the different antibody samples in terms of ADCC activity.

### Example 36

### ADCC Assays

A similar ADCC assay for IGF-1R was carried out using IGF-1R-G, IGF-1R-H and a non-antibody control using a preparation of A549 target cells and CD3/CD19 depleted PBMC effectors cells. Cell lysis was measured by Lactate dehydrogenase release according to the manufacturer's protocol (Promega). For a number of the samples the total lysis observed was in excess of the theoretical maximum and for this reason the assay was deemed a technical failure (Results not shown). Further optimisation will be required to establish a meaningful assay.

SEQUENCE LISTING

| | Sequence identifier |
|---|---|
| Polynucleotide or amino acid sequence: | (SEQ. I.D. NO) |
| 6E11 VH CDR3 | 1 |
| 6E11 VH CDR2 | 2 |
| 6E11 VH CDR1 | 3 |
| 6E11 VL CDR1 | 4 |
| 6E11 VL CDR2 | 5 |
| 6E11 VL CDR3 | 6 |
| 9C7 VL CDR2 | 7 |
| 6E11 VH | 8 |
| 6E11 VL | 9 |
| 2B9 VH | 10 |
| 2B9 VL | 11 |
| 6E11 chimera VH | 12 |
| 6E11 chimera VL | 13 |
| H0 variable | 14 |
| H1 variable | 15 |
| L0 variable | 16 |
| Biotinylated Tag sequence | 17 |
| 9C7 VH | 18 |
| 9C7 VL | 19 |
| 5G4 VH | 20 |
| 5G4 VL | 21 |
| 15D9 VH | 22 |
| 15D9 VL | 23 |
| 6E11 chimera Heavy chain | 24 |
| 6E11 chimera Light chain | 25 |
| 6E11 VH (polynucleotide sequence) | 26 |
| 6E11 VL (polynucleotide sequence) | 27 |
| 9C7 VH (polynucleotide sequence) | 28 |
| 9C7 VL (polynucleotide sequence) | 29 |
| 6E11 chimera VH (polynucleotide sequence) | 30 |
| 6E11 chimera VL (polynucleotide sequence) | 31 |
| 6E11 chimera Heavy chain (polynucleotide | 32 |
| sequence) | |
| 6E11 chimera Light chain (polynucleotide | 33 |
| sequence) | |
| H0 (polynucleotide sequence) | 34 |
| H1 (polynucleotide sequence) | 35 |
| L0 (polynucleotide sequence) | 36 |
| H0 Heavy chain | 37 |
| H1 Heavy chain | 38 |
| L0 light chain | 39 |
| H0 Heavy chain (polynucleotide sequence) | 40 |
| H1 Heavy chain (polynucleotide sequence) | 41 |
| L0 Light chain (polynucleotide sequence) | 42 |
| Campath leader | 43 |
| Human IGF-1R | 44 |
| Cynomolgus macaque IGF-1R | 45 |
| Mouse IGF-1R | 46 |
| Human IGF-1R-Fc fusion | 47 |
| Cynomolgus macaque IGF-1R-Fc fusion | 48 |
| IGF-1 | 49 |
| Tagged IGF-1 | 50 |
| IGF-2 | 51 |
| Tagged IGF-2 | 52 |
| Human Insulin receptor type B | 53 |
| H0 IgG1m(AA) Heavy chain | 54 |
| H0 IgG1m(AA) Heavy chain (polynucleotide | 55 |
| sequence) | |
| H1 IgG1m(AA) Heavy chain | 56 |
| H1 IgG1m(AA) Heavy chain (polynucleotide | 57 |
| sequence) | |
| Alternative L0 light chain (polynucleotide | 58 |
| sequence) | |
| Human Acceptor Framework Sequence-VH | 59 |
| region | |
| Human Acceptor Framework Sequence-VL | 60 |
| region | |
| H0 humanised variable domain (polynucleotide | 61 |
| sequence) | |
| L0 humanised variable domain (polynucleotide | 62 |
| sequence) | |
| Heavy chain constant region (S239D, 1332E) | 63 |
| (polynucleotide sequence) | |
| Heavy chain constant region (S239D, 1332E) | 64 |
| Heavy chain constant region (S239D, 1332E, | 65 |
| A330L) (polynucleotide sequence) | |
| Heavy chain constant region (S239D, 1332E, | 66 |
| A330L) enhanced region. | |
| H0 (S239D, 1332E) (polynucleotide sequence) | 67 |
| H0 (S239D, 1332E) | 68 |
| Alternative L0 (polynucleotide sequence) | 69 |
| Alternative H0 (polynucleotide sequence) | 70 |
| Anti-CD20 heavy chain IgG1 (Polynucleotide | 71 |
| sequence) | |
| Anti-CD20 heavy chain IgG1 with | 72 |
| S239D/I332E substitutions (Polynucleotide | |
| sequence) | |
| Anti-CD20 light chain Ck (Polynucleotide | 73 |
| sequence) | |
| Anti-CD20 heavy chain IgG1 (protein | 74 |
| sequence) | |
| Anti-CD20 heavy chain IgG1 with | 75 |
| S239D/I332E substitutions (protein | |
| sequence) | |
| Anti-CD20 light chain Ck (protein sequence) | 76 |
| His-tagged FcγRIIIa (V variant) extracellular | 77 |
| domain | |
| His-tagged FcγRIIIa (F variant) extracellular | 78 |
| domain | |

Sequence listing

SEQ ID 1:
WILYYGRSKWYFDV
SEQ ID 2:
NINPNNGGTNYNQKFKD
SEQ ID 3:
DYYMN
SEQ ID 4:
RSSQSIVQSNGDTYLE
SEQ ID 5:
RISNRFS
SEQ ID 6:
FQGSHVPYT
SEQ ID 7:
RVSNRFS
SEQ ID 8:
EVQLQQSGPELVKPGASVRISCKASGYAFTDYYMNWVKQSHGKSLEWVANINPNN
GGTNYNQKFKDKATLTVDKSSNTAYMELRSLTSEDTAVYYCARWILYYGRSKWYF
DVWGTGTTVTVSS
SEQ ID 9:
DVLMTQTPLSLPVSLGDHASISCRSSQSIVQSNGDTYLEWYLQKPGQSPKLLIYRIS
NRFSGVPDRFSGSGSGTDFTLKISRVEAEDLGVYYCFQGSHVPYTFGGGTKLEIKR
A
SEQ ID 10:
QVQLKQSGPGLVQSSQSLSITCTISGFSLTSHGIYWLRQSPGKGLEWLGVIWSGGS
ADYNAAFISRLSISKDNSKSQVFFKMNSLQADDTAIYYCARSPYYYRSSLYAMDYW
GQGTSVTVSS
SEQ ID 11:
NIVLTQSPKSMSMSIGERVTLSCKASENVGTYVSWYQQKAEQSPKLLIYGASNRHT
GVPDRFTGSGSSTDFTLTISSVQAEDLADYHCGQSYSDPLTFGAGTKLELKRA
SEQ ID 12:
EVQLQQSGPELVKPGASVRISCKASGYAFTDYYMNWVKQSHGKSLEWVANINPNN
GGTNYNQKFKDKATLTVDKSSNTAYMELRSLTSEDTAVYYCARWILYYGRSKWYF
DVWGTGTLVTVSS
SEQ ID 13:
DVLMTQTPLSLPVSLGDHASISCRSSQSIVQSNGDTYLEWYLQKPGQSPKLLIYRIS
NRFSGVPDRFSGSGSGTDFTLKISRVEAEDLGVYYCFQGSHVPYTFGGGTKLEIKR
T
SEQ ID 14:
QVQLVQSGAEVKKPGASVKVSCKASGYTFTDYYMNWVRQAPGQGLEWMGNINPN
NGGTNYNQKFKDRVTMTTDTSTSTAYMELRSLRSDDTAVYYCARWILYYGRSKWY
FDVWGRGTLVTVSS
SEQ ID 15:
QVQLVQSGAEVKKPGASVKVSCKASGYAFTDYYMNWVRQAPGQGLEWMGNINP
NNGGTNYNQKFKDRVTMTTDTSTSTAYMELRSLRSDDTAVYYCARWILYYGRSKW
YFDVWGRGTLVTVSS
SEQ ID 16:
DIVMTQSPLSLPVTPGEPASISCRSSQSIVQSNGDTYLEWYLQKPGQSPQLLIYRVS
NRFSGVPDRFSGSGSGTDFTLKISRVEAEDVGVYYCFQGSHVPYTFGQGTKLEIKR
T
SEQ ID 17:
GLNDIFEAQKIEWHE
SEQ ID 18:
EVQLQQSGPELVKPGASVRISCKASGYAFTDYYMNWVKQSHGKSLEWMANINPNN
GGTNYNQKFKDKATLTVDKSSNTAYMELRSLTSEDSAVYYCARWILYYGRSKWYF
DVWGPGTTVTVSS
SEQ ID 19:
DVLMTQSPLSLPVSLGDHASISCRSSQSIVQSNGDTYLEWYLQKPGQSPKLLIYRVS
NRFSGVPDRFSGSGSGTDFTLKISRVEAEDLGVYYCFQGSHVPYTFGGGTKLEIKR
A
SEQ ID 20:
EVQLQQSGPELVKPGASVKISCKASGYAFTDYYMNWVKQTHGRSLEWMANINPNT
GGTNYNQKFRGKATLTVDKSSTTAYMELRSLTSEDSAVYYCARWILYYGSSRWYF
DVWGTGTTVTVSS
SEQ ID 21:
DVLMTQTPLSLPVSLGDQASISCRSSQTIVHSNGNTYLEWYLQKPGQSPKLLIYKVS
NRFSGVPDRFSGSGSGTDFTLKISRVEAEDLGIYYCFQGSHVPYTFGGGTKLEIKRA
SEQ ID 22:
EVQLQQSGPELVKPGASVKISCKASGYAFTDYYMNWVKQSHGKSLEWMANINPNT
GGTNYNQKFTGKATLTVDKSSTTAYMELRSLTSEDSAVYYCTRWILYYGSSKWYFD
VWGTGTTVTVSS
SEQ ID 23:
DVLMTQTPLSLPVSLGDQASISCRSSQTIVHSNGNTYLEWYLQKPGQSPKLLIYRVS
YRFSGVPDRFSGSGSGTDFTLKISRLEAEDLGIYYCFQGSHVPYTFGGGTKLEIKRA
SEQ ID 24:
MGWSWIFFFLLSETAGVLSEVQLQQSGPELVKPGASVRISCKASGYAFTDYYMNW
VKQSHGKSLEWVANINPNNGGTNYNQKFKDKATLTVDKSSNTAYMELRSLTSEDT
AVYYCARWILYYGRSKWYFDVWGTGTLVTVSSASTKGPSVFPLAPSSKSTSGGTA
ALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMI
SRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQ
GNVFSCSVMHEALHNHYTQKSLSLSPGK
SEQ ID 25:
MKLPVRLVVLMFWIPASSSDVLMTQTPLSLPVSLGDHASISCRSSQSIVQSNGDTYL
EWYLQKPGQSPKLLIYRISNRFSGVPDRFSGSGSGTDFTLKISRVEAEDLGVYYCFQ
GSHVPYTFGGGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLS
SPVTKSFNRGEC
SEQ ID 26:
GAGGTCCAGCTGCAACAATCTGGACCTGAGCTGGTGAAGCCTGGGGCTTCAGT
GAGGATATCCTGTAAGGCTTCTGGATACGCGTTCACTGACTACTACATGAACTG
GGTGAAGCAGAGCCATGGAAAGAGCCTTGAGTGGGTGGCAAATATTAATCCCA
ACAATGGTGGTACTAACTACAACCAGAAGTTCAAGGACAAGGCCACATTGACTG
TAGACAAGTCCTCCAACACAGCCTACATGGAGCTCCGCAGTCTGACATCTGAGG
ACACTGCAGTCTATTACTGTGCAAGATGGATTCTTTACTACGGTCGTAGCAAATG
GTACTTCGATGTCTGGGGCACAGGGACCACGGTCACCGTCTCCTCG
SEQ ID 27:
GATGTTTTGATGACCCAAACTCCACTCTCCCTGCCTGTCAGTCTTGGAGATCAC
GCCTCCATCTCTTGCAGATCTAGTCAGAGTATTGTTCAAAGTAATGGAGACACCT
ATTTAGAATGGTACCTGCAGAAACCAGGCCAGTCTCCAAAGCTCCTGATCTACA
GAATTTCCAACCGATTTTCTGGGGTCCCAGACAGGTTCAGTGGCAGTGGATCAG
GGACAGATTTCACACTCAAGATCAGTAGAGTGGAGGCTGAGGATCTGGGAGTTT
ATTACTGCTTTCAGGGTTCACATGTTCCGTACACGTTCGGAGGGGGGACCAAGC
TGGAAATAAAACGGGCT
SEQ ID 28:
GAGGTCCAGCTGCAACAATCTGGACCTGAGCTGGTGAAGCCTGGGGCTTCAGT
GAGGATATCCTGTAAGGCTTCTGGATACGCGTTCACTGACTACTACATGAACTG
GGTGAAACAGAGCCATGGAAAGAGCCTTGAGTGGATGGCAAATATTAATCCCAA
CAATGGTGGTACTAACTACAACCAGAAGTTCAAGGACAAGGCCACATTGACTGT
AGACAAGTCCTCCAACACAGCCTACATGGAGCTCCGCAGTCTGACATCTGAGGA
CTCTGCAGTCTATTACTGTGCAAGATGGATTCTTTACTACGGTCGTAGCAAGTG
GTACTTCGATGTCTGGGGCCCAGGGACCACGGTCACCGTCTCCTCG
SEQ ID 29:
GATGTTTTGATGACCCAAAGTCCACTCTCCCTGCCTGTCAGTCTTGGAGATCAC
GCCTCCATCTCTTGCAGATCTAGTCAGAGCATTGTTCAAAGTAATGGAGACACC
TATTTAGAATGGTACCTGCAGAAACCAGGCCAGTCTCCAAAGCTCCTGATCTATA
GAGTTTCCAACCGATTTTCTGGGGTCCCAGACAGGTTCAGTGGCAGTGGATCAG
GGACAGATTTCACACTCAAGATCAGTAGAGTGGAGGCTGAGGATCTGGGAGTTT
ATTACTGCTTTCAGGGTTCACATGTTCCGTACACGTTCGGAGGGGGGACCAAGC
TGGAAATAAAACGGGCT
SEQ ID 30:
GAGGTCCAGCTGCAACAATCTGGACCTGAGCTGGTGAAGCCTGGGGCTTCAGT
GAGGATATCCTGTAAGGCTTCTGGATACGCGTTCACTGACTACTACATGAACTG
GGTGAAGCAGAGCCATGGAAAGAGCCTTGAGTGGGTGGCAAATATTAATCCCA
ACAATGGTGGTACTAACTACAACCAGAAGTTCAAGGACAAGGCCACATTGACTG
TAGACAAGTCCTCCAACACAGCCTACATGGAGCTCCGCAGTCTGACATCTGAGG
ACACTGCAGTCTATTACTGTGCAAGATGGATTCTTTACTACGGTCGTAGCAAATG
GTACTTCGATGTCTGGGGCACAGGGACACTAGTCACAGTCTCCTCA
SEQ ID 31:
GATGTTTTGATGACCCAAACTCCACTCTCCCTGCCTGTCAGTCTTGGAGATCAC
GCCTCCATCTCTTGCAGATCTAGTCAGAGTATTGTTCAAAGTAATGGAGACACCT
ATTTAGAATGGTACCTGCAGAAACCAGGCCAGTCTCCAAAGCTCCTGATCTACA
GAATTTCCAACCGATTTTCTGGGGTCCCAGACAGGTTCAGTGGCAGTGGATCAG
GGACAGATTTCACACTCAAGATCAGTAGAGTGGAGGCTGAGGATCTGGGAGTTT
ATTACTGCTTTCAGGGTTCACATGTTCCGTACACGTTCGGAGGGGGGACCAAGC
TGGAAATAAAACGTACG
SEQ ID 32:
ATGGGATGGAGCTGGATCTTTTTCTTCCTCCTGTCAGAAACTGCAGGTGTCCTC
TCTGAGGTCCAGCTGCAACAATCTGGACCTGAGCTGGTGAAGCCTGGGGCTTC
AGTGAGGATATCCTGTAAGGCTTCTGGATACGCGTTCACTGACTACTACATGAA
CTGGGTGAAGCAGAGCCATGGAAAGAGCCTTGAGTGGGTGGCAAATATTAATC
CCAACAATGGTGGTACTAACTACAACCAGAAGTTCAAGGACAAGGCCACATTGA
CTGTAGACAAGTCCTCCAACACAGCCTACATGGAGCTCCGCAGTCTGACATCTG
AGGACACTGCAGTCTATTACTGTGCAAGATGGATTCTTTACTACGGTCGTAGCA
AATGGTACTTCGATGTCTGGGGCACAGGGACACTAGTCACAGTCTCCTCAGCCT
CCACCAAGGGCCCATCGGTCTTCCCCCTGGCACCCTCCTCCAAGAGCACCTCT
GGGGGCACAGCGGCCCTGGGCTGCCTGGTCAAGGACTACTTCCCCGAACCGG
TGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCC
CTCCAGCAGCTTGGGCACCCAGACCTACATCTGCAACGTGAATCACAAGCCCA
GCAACACCAAGGTGGACAAGAAAGTTGAGCCCAAATCTTGTGACAAAACTCACA
CATGCCCACCGTGCCCAGCACCTGAACTCCTGGGGGGACCGTCAGTCTTCCTC
TTCCCCCCAAAACCCAAGGACACCCTCATGATCTCCCGGACCCCTGAGGTCACA
TGCGTGGTGGTGGACGTGAGCCACGAAGACCCTGAGGTCAAGTTCAACTGGTA
CGTGGACGGCGTGGAGGTGCATAATGCCAAGACAAAGCCGCGGGAGGAGCAG
TACAACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAGCCC
CCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGAC
CTGCCTGGTCAAAGGCTTCTATCCCAGCGACATCGCCGTGGAGTGGGAGAGCA
ATGGGCAGCCGGAGAACAACTACAAGACCACGCCTCCCGTGCTGGACTCCGAC
GGCTCCTTCTTCCTCTACAGCAAGCTCACCGTGGACAAGAGCAGGTGGCAGCA
GGGGAACGTCTTCTCATGCTCCGTGATGCATGAGGCTCTGCACAACCACTACAC
GCAGAAGAGCCTCTCCCTGTCTCCGGGTAAATGA
SEQ ID 33:
ATGAAGTTGCCTGTTCGGCTCGTGGTGCTGATGTTCTGGATTCCTGCTTCCAGC
AGTGATGTTTTGATGACCCAAACTCCACTCTCCCTGCCTGTCAGTCTTGGAGAT
CACGCCTCCATCTCTTGCAGATCTAGTCAGAGTATTGTTCAAAGTAATGGAGACA
CCTATTTAGAATGGTACCTGCAGAAACCAGGCCAGTCTCCAAAGCTCCTGATCT
ACAGAATTTCCAACCGATTTTCTGGGGTCCCAGACAGGTTCAGTGGCAGTGGAT
CAGGGACAGATTTCACACTCAAGATCAGTAGAGTGGAGGCTGAGGATCTGGGA
GTTTATTACTGCTTTCAGGGTTCACATGTTCCGTACACGTTCGGAGGGGGGACC
AAGCTGGAAATAAAACGTACGGTGGCTGCACCATCTGTCTTCATCTTCCCGCCA
TCTGATGAGCAGTTGAAATCTGGAACTGCCTCTGTTGTGTGCCTGCTGAATAAC
TTCTATCCCAGAGAGGCCAAAGTACAGTGGAAGGTGGACAACGCCCTCCAATC
GGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTC
TACGCCTGCGAAGTCACCCATCAGGGCCTGAGCTCGCCCGTCACAAAGAGCTT
CAACAGGGGAGAGTGTTAG
SEQ ID 34:
CAGGTCCAGCTGGTGCAGAGCGGCGCAGAGGTGAAGAAGCCCGGAGCTAGCG
TCAAGGTCTCCTGCAAGGCTTCAGGCTACACATTCACCGACTACTACATGAACT
GGGTGAGACAGGCTCCAGGACAGGGCCTCGAGTGGATGGGCAACATCAACCC
CAACAATGGCGGGACAAACTACAACCAGAAGTTCAAGGATCGCGTGACCATGA
CCACCGACACTAGCACCTCAACAGCCTACATGGAGCTGAGGTCTCTGCGGAGC
GATGACACTGCCGTGTACTACTGTGCCAGGTGGATTCTGTACTACGGGAGGAG
CAAGTGGTACTTCGACGTCTGGGGAAGAGGGACACTAGTGACCGTGAGCAGC
SEQ ID 35:
CAGGTCCAGCTGGTGCAGAGCGGCGCAGAGGTGAAGAAGCCCGGAGCTAGCG
TCAAGGTCTCCTGCAAGGCTTCAGGCTACGCCTTCACCGACTACTACATGAACT
GGGTGAGACAGGCTCCAGGACAGGGCCTCGAGTGGATGGGCAACATCAACCC
CAACAATGGCGGGACAAACTACAACCAGAAGTTCAAGGATCGCGTGACCATGA
CCACCGACACTAGCACCTCAACAGCCTACATGGAGCTGAGGTCTCTGCGGAGC
GATGACACTGCCGTGTACTACTGTGCCAGGTGGATTCTGTACTACGGGAGGAG
CAAGTGGTACTTCGACGTCTGGGGAAGAGGGACACTAGTGACCGTGAGCAGC
SEQ ID 36:
GACATCGTCATGACCCAGAGCCCACTGTCACTCCCCGTGACACCCGGAGAGCC
CGCTAGCATCAGCTGTAGAAGCTCCCAGAGCATCGTGCAGTCTAACGGCGATA
CCTACCTCGAGTGGTACCTGCAGAAGCCCGGACAGTCTCCTCAGCTCCTGATTT
ACCGCGTCAGCAATCGCTTTTCCGGGGTGCCTGATCGGTTTAGCGGCTCAGGA
AGCGGAACCGACTTCACCCTGAAGATCTCAAGGGTGGAGGCTGAGGATGTGGG
CGTGTACTACTGCTTCCAGGGATCTCACGTGCCTTACACCTTCGGACAGGGCAC
AAAGCTCGAGATTAAGCGTACG
SEQ ID 37:
MGWSCIILFLVATATGVHSQVQLVQSGAEVKKPGASVKVSCKASGYTFTDYYMNW
VRQAPGQGLEWMGNINPNNGGTNYNQKFKDRVTMTTDTSTSTAYMELRSLRSDD
TAVYYCARWILYYGRSKWYFDVWGRGTLVTVSSASTKGPSVFPLAPSSKSTSGGT
AALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQ
QGNVFSCSVMHEALHNHYTQKSLSLSPGK
SEQ ID 38:
MGWSCIILFLVATATGVHSQVQLVQSGAEVKKPGASVKVSCKASGYAFTDYYMNW
VRQAPGQGLEWMGNINPNNGGTNYNQKFKDRVTMTTDTSTSTAYMELRSLRSDD
TAVYYCARWILYYGRSKWYFDVWGRGTLVTVSSASTKGPSVFPLAPSSKSTSGGT
AALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQ
QGNVFSCSVMHEALHNHYTQKSLSLSPGK
SEQ ID 39:
MGWSCIILFLVATATGVHSDIVMTQSPLSLPVTPGEPASISCRSSQSIVQSNGDTYLE
WYLQKPGQSPQLLIYRVSNRFSGVPDRFSGSGSGTDFTLKISRVEAEDVGVYYCFQ
GSHVPYTFGQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLS
SPVTKSFNRGEC
SEQ ID 40:
ATGGGATGGTCCTGTATCATCCTGTTTCTGGTGGCCACAGCAACTGGCGTGCAC
TCTCAGGTCCAGCTGGTGCAGAGCGGCGCAGAGGTGAAGAAGCCCGGAGCTA
GCGTCAAGGTCTCCTGCAAGGCTTCAGGCTACACATTCACCGACTACTACATGA
ACTGGGTGAGACAGGCTCCAGGACAGGGCCTCGAGTGGATGGGCAACATCAAC
CCCAACAATGGCGGGACAAACTACAACCAGAAGTTCAAGGATCGCGTGACCAT
GACCACCGACACTAGCACCTCAACAGCCTACATGGAGCTGAGGTCTCTGCGGA
GCGATGACACTGCCGTGTACTACTGTGCCAGGTGGATTCTGTACTACGGGAGG
AGCAAGTGGTACTTCGACGTCTGGGGAAGAGGGACACTAGTGACCGTGTCCAG
CGCCAGCACCAAGGGCCCCAGCGTGTTCCCCCTGGCCCCCAGCAGCAAGAGC
ACCAGCGGCGGCACAGCCGCCCTGGGCTGCCTGGTGAAGGACTACTTCCCCG
AACCGGTGACCGTGTCCTGGAACAGCGGAGCCCTGACCAGCGGCGTGCACAC
CTTCCCCGCCGTGCTGCAGAGCAGCGGCCTGTACAGCCTGAGCAGCGTGGTG
ACCGTGCCCAGCAGCAGCCTGGGCACCCAGACCTACATCTGTAACGTGAACCA
CAAGCCCAGCAACACCAAGGTGGACAAGAAGGTGGAGCCCAAGAGCTGTGACA
AGACCCACACCTGCCCCCCCTGCCCTGCCCCCGAGCTGCTGGGAGGCCCCAG
CGTGTTCCTGTTCCCCCCCAAGCCTAAGGACACCCTGATGATCAGCAGAACCCC
CGAGGTGACCTGTGTGGTGGTGGATGTGAGCCACGAGGACCCTGAGGTGAAGT
TCAACTGGTACGTGGACGGCGTGGAGGTGCACAATGCCAAGACCAAGCCCAGG
GAGGAGCAGTACAACAGCACCTACCGGGTGGTGTCCGTGCTGACCGTGCTGCA
CCAGGATTGGCTGAACGGCAAGGAGTACAAGTGTAAGGTGTCCAACAAGGCCC
TGCCTGCCCCTATCGAGAAAACCATCAGCAAGGCCAAGGGCCAGCCCAGAGAG
CCCCAGGTGTACACCCTGCCCCCTAGCAGAGATGAGCTGACCAAGAACCAGGT
GTCCCTGACCTGCCTGGTGAAGGGCTTCTACCCCAGCGACATCGCCGTGGAGT
GGGAGAGCAACGGCCAGCCCGAGAACAACTACAAGACCACCCCCCCTGTGCTG
GACAGCGATGGCAGCTTCTTCCTGTACAGCAAGCTGACCGTGGACAAGAGCAG
ATGGCAGCAGGGCAACGTGTTCAGCTGCTCCGTGATGCACGAGGCCCTGCACA
ATCACTACACCCAGAAGAGCCTGAGCCTGTCCCCTGGCAAGTGA
SEQ ID 41:
ATGGGATGGTCCTGTATCATCCTGTTTCTGGTGGCCACAGCAACTGGCGTGCAC
TCTCAGGTCCAGCTGGTGCAGAGCGGCGCAGAGGTGAAGAAGCCCGGAGCTA
GCGTCAAGGTCTCCTGCAAGGCTTCAGGCTACGCCTTCACCGACTACTACATGA
ACTGGGTGAGACAGGCTCCAGGACAGGGCCTCGAGTGGATGGGCAACATCAAC
CCCAACAATGGCGGGACAAACTACAACCAGAAGTTCAAGGATCGCGTGACCAT
GACCACCGACACTAGCACCTCAACAGCCTACATGGAGCTGAGGTCTCTGCGGA
GCGATGACACTGCCGTGTACTACTGTGCCAGGTGGATTCTGTACTACGGGAGG
AGCAAGTGGTACTTCGACGTCTGGGGAAGAGGGACACTAGTGACCGTGTCCAG
CGCCAGCACCAAGGGCCCCAGCGTGTTCCCCCTGGCCCCCAGCAGCAAGAGC
ACCAGCGGCGGCACAGCCGCCCTGGGCTGCCTGGTGAAGGACTACTTCCCCG
AACCGGTGACCGTGTCCTGGAACAGCGGAGCCCTGACCAGCGGCGTGCACAC
CTTCCCCGCCGTGCTGCAGAGCAGCGGCCTGTACAGCCTGAGCAGCGTGGTG
ACCGTGCCCAGCAGCAGCCTGGGCACCCAGACCTACATCTGTAACGTGAACCA
CAAGCCCAGCAACACCAAGGTGGACAAGAAGGTGGAGCCCAAGAGCTGTGACA
AGACCCACACCTGCCCCCCCTGCCCTGCCCCCGAGCTGCTGGGAGGCCCCAG
CGTGTTCCTGTTCCCCCCCAAGCCTAAGGACACCCTGATGATCAGCAGAACCCC
CGAGGTGACCTGTGTGGTGGTGGATGTGAGCCACGAGGACCCTGAGGTGAAGT
TCAACTGGTACGTGGACGGCGTGGAGGTGCACAATGCCAAGACCAAGCCCAGG
GAGGAGCAGTACAACAGCACCTACCGGGTGGTGTCCGTGCTGACCGTGCTGCA
CCAGGATTGGCTGAACGGCAAGGAGTACAAGTGTAAGGTGTCCAACAAGGCCC
TGCCTGCCCCTATCGAGAAAACCATCAGCAAGGCCAAGGGCCAGCCCAGAGAG
CCCCAGGTGTACACCCTGCCCCCTAGCAGAGATGAGCTGACCAAGAACCAGGT
GTCCCTGACCTGCCTGGTGAAGGGCTTCTACCCCAGCGACATCGCCGTGGAGT
GGGAGAGCAACGGCCAGCCCGAGAACAACTACAAGACCACCCCCCCTGTGCTG
GACAGCGATGGCAGCTTCTTCCTGTACAGCAAGCTGACCGTGGACAAGAGCAG
ATGGCAGCAGGGCAACGTGTTCAGCTGCTCCGTGATGCACGAGGCCCTGCACA
ATCACTACACCCAGAAGAGCCTGAGCCTGTCCCCTGGCAAGTGA
SEQ ID 42:
ATGGGATGGTCCTGCATCATCCTGTTCCTGGTGGCAACTGCCACTGGAGTCCAC
TCCGACATCGTCATGACCCAGAGCCCACTGTCACTCCCCGTGACACCCGGAGA
GCCCGCTAGCATCAGCTGTAGAAGCTCCCAGAGCATCGTGCAGTCTAACGGCG
ATACCTACCTCGAGTGGTACCTGCAGAAGCCCGGACAGTCTCCTCAGCTCCTGA
TTTACCGCGTCAGCAATCGCTTTTCCGGGGTGCCTGATCGGTTTAGCGGCTCAG
GAAGCGGAACCGACTTCACCCTGAAGATCTCAAGGGTGGAGGCTGAGGATGTG
GGCGTGTACTACTGCTTCCAGGGATCTCACGTGCCTTACACCTTCGGACAGGG
CACAAAGCTCGAGATTAAGCGTACGGTGGCCGCCCCCAGCGTGTTCATCTTCC
CCCCCAGCGATGAGCAGCTGAAGAGCGGCACCGCCAGCGTGGTGTGTCTGCT
GAACAACTTCTACCCCCGGGAGGCCAAGGTGCAGTGGAAGGTGGACAATGCCC
TGCAGAGCGGCAACAGCCAGGAGAGCGTGACCGAGCAGGACAGCAAGGACTC
CACCTACAGCCTGAGCAGCACCCTGACCCTGAGCAAGGCCGACTACGAGAAGC
ACAAGGTGTACGCCTGTGAGGTGACCCACCAGGGCCTGTCCAGCCCCGTGACC
AAGAGCTTCAACCGGGGCGAGTGCTGA
SEQ ID 43:
MGWSCIILFLVATATGVHS
SEQ ID 44:
MKSGSGGGSPTSLWGLLFLSAALSLWPTSGEICGPGIDIRNDYQQLKRLENCTVIEG
YLHILLISKAEDYRSYRFPKLTVITEYLLLFRVAGLESLGDLFPNLTVIRGWKLFYNYAL
VIFEMTNLKDIGLYNLRNITRGAIRIEKNADLCYLSTVDWSLILDAVSNNYIVGNKPPK
ECGDLCPGTMEEKPMCEKTTINNEYNYRCWTTNRCQKMCPSTCGKRACTENNEC
CHPECLGSCSAPDNDTACVACRHYYYAGVCVPACPPNTYRFEGWRCVDRDFCAN
ILSAESSDSEGFVIHDGECMQECPSGFIRNGSQSMYCIPCEGPCPKVCEEEKKTKTI
DSVTSAQMLQGCTIFKGNLLINIRRGNNIASELENFMGLIEVVTGYVKIRHSHALVSLS
FLKNLRLILGEEQLEGNYSFYVLDNQNLQQLWDWDHRNLTIKAGKMYFAFNPKLCV
SEIYRMEEVTGTKGRQSKGDINTRNNGERASCESDVLHFTSTTTSKNRIIITWHRYR
PPDYRDLISFTVYYKEAPFKNVTEYDGQDACGSNSWNMVDVDLPPNKDVEPGILLH
GLKPWTQYAVYVKAVTLTMVENDHIRGAKSEILYIRTNASVPSIPLDVLSASNSSSQL
IVKWNPPSLPNGNLSYYIVRWQRQPQDGYLYRHNYCSKDKIPIRKYADGTIDIEEVT
ENPKTEVCGGEKGPCCACPKTEAEKQAEKEEAEYRKVFENFLHNSIFVPRPERKRR
DVMQVANTTMSSRSRNTTAADTYNITDPEELETEYPFFESRVDNKERTVISNLRPFT
LYRIDIHSCNHEAEKLGCSASNFVFARTMPAEGADDIPGPVTWEPRPENSIFLKWPE
PENPNGLILMYEIKYGSQVEDQRECVSRQEYRKYGGAKLNRLNPGNYTARIQATSL
SGNGSWTDPVFFYVQAKTGYENFIHLIIALPVAVLLIVGGLVIMLYVFHRKRNNSRLG
NGVLYASVNPEYFSAADVYVPDEWEVAREKITMSRELGQGSFGMVYEGVAKGVVK
DEPETRVAIKTVNEAASMRERIEFLNEASVMKEFNCHHVVRLLGVVSQGQPTLVIME
LMTRGDLKSYLRSLRPEMENNPVLAPPSLSKMIQMAGEIADGMAYLNANKFVHRDL
AARNCMVAEDFTVKIGDFGMTRDIYETDYYRKGGKGLLPVRWMSPESLKDGVFTT
YSDVWSFGVVLWEIATLAEQPYQGLSNEQVLRFVMEGGLLDKPDNCPDMLFELMR
MCWQYNPKMRPSFLEIISSIKEEMEPGFREVSFYYSEENKLPEPEELDLEPENMES
VPLDPSASSSSLPLPDRHSGHKAENGPGPGVLVLRASFDERQPYAHMNGGRKNE
RALPLPQSSTC
SEQ ID 45:
MKSGSGGGSPTSLWGLLFLSAALSLWPTSGEICGPGIDIRNDYQQLKRLENCTVIEG
YLHILLISKAEDYRSYRFPKLTVITEYLLLFRVAGLESLGDLFPNLTVIRGWKLFYNYAL
VIFEMTNLKDIGLYNLRNITRGAIRIEKNADLCYLSTVDWSLILDAVSNNYIVGNKPPK
ECGDLCPGTMEEKPMCEKTTINNEYNYRCWTTNRCQKMCPSACGKRACTENNEC
CHPECLGSCSAPDNDTACVACRHYYYAGVCVPACPPNTYRFEGWRCVDRDFCAN
ILSAESSDSEGFVIHDGECMQECPSGFIRNGSQSMYCIPCEGPCPKVCEEEKKTKTI
DSVTSAQMLQGCTIFKGNLLINIRRGNNIASELENFMGLIEVVTGYVKIRHSHALVSLS
FLKNLRLILGEEQLEGNYSFYVLDNQNLQQLWDWDHRNLTIKAGKMYFAFNPKLCV
SEIYRMEEVTGTKGRQSKGDINTRNNGERASCESDVLHFTSTTTWKNRIIITWHRYR
PPDYRDLISFTVYYKEAPFKNVTEYDGQDACGSNSWNMVDVDLPPNKDVEPGILLH
GLKPWTQYAVYVKAVTLTMVENDHIRGAKSEILYIRTNASVPSIPLDVLSASNSSSQL
IVKWNPPSLPNGNLSYYIVRWQRQPQDGYLYRHNYCSKDKIPIRKYADGTIDIEEVT
ENPKTEVCGGEKGPCCACPKTEAEKQAEKEEAEYRKVFENFLHNSIFVPRPERKRR
DVMQVANTTMSSRSRNTTAADTYNITDLEELETEYPFFESRVDNKERTVISNLRPFT
LYRIDIHSCNHEAEKLGCSASNFVFARTMPAEGADDIPGPVTWEPRPENSIFLKWPE
PENPNGLILMYEIKYGSQVEDQRECVSRQEYRKYGGAKLNRLNPGNYTARIQATSL
SGNGSWTDPVFFYVQAKTGYENFIHLIIALPVAVLLIVGGLVIMLYVFHRKRNNSRLG
NGVLYASVNPEYFSAADVYVPDEWEVAREKITMSRELGQGSFGMVYEGVAKGVVK
DEPETRVAIKTVNEAASMRERIEFLNEASVMKEFNCHHVVRLLGVVSQGQPTLVIME
LMTRGDLKSYLRSLRPEMENNPVLAPPSLSKMIQMAGEIADGMAYLNANKFVHRDL
AARNCMVAEDFTVKIGDFGMTRDIYETDYYRKGGKGLLPVRWMSPESLKDGVFTT
YSDVWSFGVVLWEIATLAEQPYQGLSNEQVLRFVMEGGLLDKPDNCPDMLFELMR
MCWQYNPKMRPSFLEIISSIKDEMEPGFREVSFYYSEENKLPEPEELDLEPENMES
VPLDPSASSSSLPLPDRHSGHKAENGPGPGVLVLRASFDERQPYAHMNGGRKNE
RALPLPQSSTC
SEQ ID 46:
MKSGSGGGSPTSLWGLVFLSAALSLWPTSGEICGPGIDIRNDYQQLKRLENCTVIE
GFLHILLISKAEDYRSYRFPKLTVITEYLLLFRVAGLESLGDLFPNLTVIRGWKLFYNY
ALVIFEMTNLKDIGLYNLRNITRGAIRIEKNADLCYLSTIDWSLILDAVSNNYIVGNKPP
KECGDLCPGTLEEKPMCEKTTINNEYNYRCWTTNRCQKMCPSVCGKRACTENNE
CCHPECLGSCHTPDDNTTCVACRHYYYKGVCVPACPPGTYRFEGWRCVDRDFCA
NIPNAESSDSDGFVIHDDECMQECPSGFIRNSTQSMYCIPCEGPCPKVCGDEEKKT
KTIDSVTSAQMLQGCTILKGNLLINIRRGNNIASELENFMGLIEVVTGYVKIRHSHALV
SLSFLKNLRLILGEEQLEGNYSFYVLDNQNLQQLWDWNHRNLTVRSGKMYFAFNP
KLCVSEIYRMEEVTGTKGRQSKGDINTRNNGERASCESDVLRFTSTTTWKNRIIITW
HRYRPPDYRDLISFTVYYKEAPFKNVTEYDGQDACGSNSWNMVDVDLPPNKEGEP
GILLHGLKPWTQYAVYVKAVTLTMVENDHIRGAKSEILYIRTNASVPSIPLDVLSASN
SSSQLIVKWNPPTLPNGNLSYYIVRWQRQPQDGYLYRHNYCSKDKIPIRKYADGTID
VEEVTENPKTEVCGGDKGPCCACPKTEAEKQAEKEEAEYRKVFENFLHNSIFVPRP
ERRRRDVMQVANTTMSSRSRNTTVADTYNITDPEEFETEYPFFESRVDNKERTVIS
NLRPFTLYRIDIHSCNHEAEKLGCSASNFVFARTMPAEGADDIPGPVTWEPRPENSI
FLKWPEPENPNGLILMYEIKYGSQVEDQRECVSRQEYRKYGGAKLNRLNPGNYTA
RIQATSLSGNGSWTDPVFFYVPAKTTYENFMHLIIALPVAILLIVGGLVIMLYVFHRKR
NNSRLGNGVLYASVNPEYFSAADVYVPDEWEVAREKITMNRELGQGSFGMVYEGV
AKGVVKDEPETRVAIKTVNEAASMRERIEFLNEASVMKEFNCHHVVRLLGVVSQGQ
PTLVIMELMTRGDLKSYLRSLRPEVEQNNLVLIPPSLSKMIQMAGEIADGMAYLNAN
KFVHRDLAARNCMVAEDFTVKIGDFGMTRDIYETDYYRKGGKGLLPVRWMSPESL
KDGVFTTHSDVWSFGVVLWEIATLAEQPYQGLSNEQVLRFVMEGGLLDKPDNCPD
MLFELMRMCWQYNPKMRPSFLEIIGSIKDEMEPSFQEVSFYYSEENKPPEPEELEM
ELEMEPENMESVPLDPSASSASLPLPERHSGHKAENGPGPGVLVLRASFDERQPY
AHMNGGRANERALPLPQSSTC
SEQ ID 47:
MKSGSGGGSPTSLWGLLFLSAALSLWPTSGEICGPGIDIRNDYQQLKRLENCTVIEG
YLHILLISKAEDYRSYRFPKLTVITEYLLLFRVAGLESLGDLFPNLTVIRGWKLFYNYAL
VIFEMTNLKDIGLYNLRNITRGAIRIEKNADLCYLSTVDWSLILDAVSNNYIVGNKPPK
ECGDLCPGTMEEKPMCEKTTINNEYNYRCWTTNRCQKMCPSTCGKRACTENNEC
CHPECLGSCSAPDNDTACVACRHYYYAGVCVPACPPNTYRFEGWRCVDRDFCAN
ILSAESSDSEGFVIHDGECMQECPSGFIRNGSQSMYCIPCEGPCPKVCEEEKKTKTI
DSVTSAQMLQGCTIFKGNLLINIRRGNNIASELENFMGLIEVVTGYVKIRHSHALVSLS
FLKNLRLILGEEQLEGNYSFYVLDNQNLQQLWDWDHRNLTIKAGKMYFAFNPKLCV
SEIYRMEEVTGTKGRQSKGDINTRNNGERASCESDVLHFTSTTTSKNRIIITWHRYR
PPDYRDLISFTVYYKEAPFKNVTEYDGQDACGSNSWNMVDVDLPPNKDVEPGILLH
GLKPWTQYAVYVKAVTLTMVENDHIRGAKSEILYIRTNASVPSIPLDVLSASNSSSQL
IVKWNPPSLPNGNLSYYIVRWQRQPQDGYLYRHNYCSKDKIPIRKYADGTIDIEEVT
ENPKTEVCGGEKGPCCACPKTEAEKQAEKEEAEYRKVFENFLHNSIFVPRPERKRR
DVMQVANTTMSSRSRNTTAADTYNITDPEELETEYPFFESRVDNKERTVISNLRPFT
LYRIDIHSCNHEAEKLGCSASNFVFARTMPAEGADDIPGPVTWEPRPENSIFLKWPE
PENPNGLILMYEIKYGSQVEDQRECVSRQEYRKYGGAKLNRLNPGNYTARIQATSL
SGNGSWTDPVFFYVQAKTGYENFIHAAAIEGRSGSCDKTHTCPPCPAPELLGGPSV
FLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSK
LTVDKSRWQQGNVFSCSVMHEALHNHYTQKSLSLSPGKLRRASLG
SEQ ID 48:
MKSGSGGGSPTSLWGLLFLSAALSLWPTSGEICGPGIDIRNDYQQLKRLENCTVIEG
YLHILLISKAEDYRSYRFPKLTVITEYLLLFRVAGLESLGDLFPNLTVIRGWKLFYNYAL
VIFEMTNLKDIGLYNLRNITRGAIRIEKNADLCYLSTVDWSLILDAVSNNYIVGNKPPK
ECGDLCPGTMEEKPMCEKTTINNEYNYRCWTTNRCQKMCPSACGKRACTENNEC
CHPECLGSCSAPDNDTACVACRHYYYAGVCVPACPPNTYRFEGWRCVDRDFCAN
ILSAESSDSEGFVIHDGECMQECPSGFIRNGSQSMYCIPCEGPCPKVCEEEKKTKTI
DSVTSAQMLQGCTIFKGNLLINIRRGNNIASELENFMGLIEVVTGYVKIRHSHALVSLS
FLKNLRLILGEEQLEGNYSFYVLDNQNLQQLWDWDHRNLTIKAGKMYFAFNPKLCV
SEIYRMEEVTGTKGRQSKGDINTRNNGERASCESDVLHFTSTTTWKNRIIITWHRYR
PPDYRDLISFTVYYKEAPFKNVTEYDGQDACGSNSWNMVDVDLPPNKDVEPGILLH
GLKPWTQYAVYVKAVTLTMVENDHIRGAKSEILYIRTNASVPSIPLDVLSASNSSSQL
IVKWNPPSLPNGNLSYYIVRWQRQPQDGYLYRHNYCSKDKIPIRKYADGTIDIEEVT
ENPKTEVCGGEKGPCCACPKTEAEKQAEKEEAEYRKVFENFLHNSIFVPRPERKRR
DVMQVANTTMSSRSRNTTAADTYNITDLEELETEYPFFESRVDNKERTVISNLRPFT
LYRIDIHSCNHEAEKLGCSASNFVFARTMPAEGADDIPGPVTWEPRPENSIFLKWPE
PENPNGLILMYEIKYGSQVEDQRECVSRQEYRKYGGAKLNRLNPGNYTARIQATSL
SGNGSWTDPVFFYVQAKTGYENFIHAAAIEGRSGSCDKTHTCPPCPAPELLGGPSV
FLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSK
LTVDKSRWQQGNVFSCSVMHEALHNHYTQKSLSLSPGKLRRASLG
SEQ ID 49:
MGPETLCGAELVDALQFVCGDRGFYFNKPTGYGSSSRRAPQTGIVDECCFRSCDL
RRLEMYCAPLKPAKSA
SEQ ID 50:
MGPETLCGAELVDALQFVCGDRGFYFNKPTGYGSSSRRAPQTGIVDECCFRSCDL
RRLEMYCAPLKPAKSAGLNDIFEAQKIEWHE
SEQ ID 51:
MAYRPSETLCGGELVDTLQFVCGDRGFYFSRPASRVSRRSRGIVEECCFRSCDLA
LLETYCATPAKSE
SEQ ID 52:
MAYRPSETLCGGELVDTLQFVCGDRGFYFSRPASRVSRRSRGIVEECCFRSCDLA
LLETYCATPAKSEGLNDIFEAQKIEWHE
SEQ ID 53:
MGTGGRRGAAAAPLLVAVAALLLGAAGHLYPGEVCPGMDIRNNLTRLHELENCSVI
EGHLQILLMFKTRPEDFRDLSFPKLIMITDYLLLFRVYGLESLKDLFPNLTVIRGSRLF
FNYALVIFEMVHLKELGLYNLMNITRGSVRIEKNNELCYLATIDWSRILDSVEDNYIVL
NKDDNEECGDICPGTAKGKTNCPATVINGQFVERCWTHSHCQKVCPTICKSHGCT
AEGLCCHSECLGNCSQPDDPTKCVACRNFYLDGRCVETCPPPYYHFQDWRCVNF
SFCQDLHHKCKNSRRQGCHQYVIHNNKCIPECPSGYTMNSSNLLCTPCLGPCPKV
CHLLEGEKTIDSVTSAQELRGCTVINGSLIINIRGGNNLAAELEANLGLIEEISGYLKIR
RSYALVSLSFFRKLRLIRGETLEIGNYSFYALDNQNLRQLWDWSKHNLTITQGKLFF
HYNPKLCLSEIHKMEEVSGTKGRQERNDIALKTNGDQASCENELLKFSYIRTSFDKIL
LRWEPYWPPDFRDLLGFMLFYKEAPYQNVTEFDGQDACGSNSWTVVDIDPPLRSN
DPKSQNHPGWLMRGLKPWTQYAIFVKTLVTFSDERRTYGAKSDIIYVQTDATNPSV
PLDPISVSNSSSQIILKWKPPSDPNGNITHYLVFWERQAEDSELFELDYCLKGLKLPS
RTWSPPFESEDSQKHNQSEYEDSAGECCSCPKTDSQILKELEESSFRKTFEDYLHN
VVFVPRKTSSGTGAEDPRPSRKRRSLGDVGNVTVAVPTVAAFPNTSSTSVPTSPEE
HRPFEKVVNKESLVISGLRHFTGYRIELQACNQDTPEERCSVAAYVSARTMPEAKA
DDIVGPVTHEIFENNVVHLMWQEPKEPNGLIVLYEVSYRRYGDEELHLCVSRKHFAL
ERGCRLRGLSPGNYSVRIRATSLAGNGSWTEPTYFYVTDYLDVPSNIAKIIIGPLIFVF
LFSVVIGSIYLFLRKRQPDGPLGPLYASSNPEYLSASDVFPCSVYVPDEWEVSREKI
TLLRELGQGSFGMVYEGNARDIIKGEAETRVAVKTVNESASLRERIEFLNEASVMKG
FTCHHVVRLLGVVSKGQPTLVVMELMAHGDLKSYLRSLRPEAENNPGRPPPTLQE
MIQMAAEIADGMAYLNAKKFVHRDLAARNCMVAHDFTVKIGDFGMTRDIYETDYYR
KGGKGLLPVRWMAPESLKDGVFTTSSDMWSFGVVLWEITSLAEQPYQGLSNEQVL
KFVMDGGYLDQPDNCPERVTDLMRMCWQFNPNMRPTFLEIVNLLKDDLHPSFPEV
SFFHSEENKAPESEELEMEFEDMENVPLDRSSHCQREEAGGRDGGSSLGFKRSY
EEHIPYTHMNGGKKNGRILTLPRSNPS
SEQ ID 54:
MGWSCIILFLVATATGVHSQVQLVQSGAEVKKPGASVKVSCKASGYTFTDYYMNW
VRQAPGQGLEWMGNINPNNGGTNYNQKFKDRVTMTTDTSTSTAYMELRSLRSDD
TAVYYCARWILYYGRSKWYFDVWGRGTLVTVSSASTKGPSVFPLAPSSKSTSGGT
AALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELAGAPSVFLFPPKPKDTL
MISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQ
QGNVFSCSVMHEALHNHYTQKSLSLSPGK
SEQ ID 55:
ATGGGATGGTCCTGTATCATCCTGTTTCTGGTGGCCACAGCAACTGGCGTGCAC
TCTCAGGTCCAGCTGGTGCAGAGCGGCGCAGAGGTGAAGAAGCCCGGAGCTA
GCGTCAAGGTCTCCTGCAAGGCTTCAGGCTACACATTCACCGACTACTACATGA
ACTGGGTGAGACAGGCTCCAGGACAGGGCCTCGAGTGGATGGGCAACATCAAC
CCCAACAATGGCGGGACAAACTACAACCAGAAGTTCAAGGATCGCGTGACCAT
GACCACCGACACTAGCACCTCAACAGCCTACATGGAGCTGAGGTCTCTGCGGA
GCGATGACACTGCCGTGTACTACTGTGCCAGGTGGATTCTGTACTACGGGAGG
AGCAAGTGGTACTTCGACGTCTGGGGAAGAGGGACACTAGTGACCGTGAGCAG
CGCCAGCACCAAGGGCCCCAGCGTGTTCCCCCTGGCCCCCAGCAGCAAGAGC
ACCAGCGGCGGCACAGCCGCCCTGGGCTGCCTGGTGAAGGACTACTTCCCCG
AGCCCGTGACCGTGTCCTGGAACAGCGGAGCCCTGACAAGCGGGGTGCACAC
CTTCCCCGCCGTGCTGCAGAGCAGCGGCCTGTACAGCCTGAGCAGCGTGGTG
ACAGTGCCCAGCAGCAGCCTGGGCACCCAGACCTACATCTGCAACGTGAACCA
CAAGCCCAGCAACACCAAGGTGGACAAGAAGGTGGAGCCCAAGAGCTGCGAC
AAGACCCACACCTGCCCCCCCTGCCCTGCCCCTGAACTGGCCGGAGCCCCCTC
CGTGTTCCTGTTCCCCCCCAAGCCCAAGGACACCCTGATGATCAGCCGGACCC
CCGAGGTGACCTGCGTGGTGGTGGACGTGAGCCACGAGGACCCTGAGGTGAA
GTTCAATTGGTACGTGGACGGCGTGGAGGTGCACAACGCCAAGACCAAGCCCC
GGGAGGAACAGTACAACAGCACCTACCGGGTGGTGTCCGTGCTGACCGTGCTG
CACCAGGACTGGCTGAACGGCAAAGAATACAAGTGCAAGGTGTCCAACAAGGC
CCTGCCTGCCCCCATCGAGAAAACCATCAGCAAGGCCAAGGGCCAGCCCAGG
GAACCCCAGGTGTACACCCTGCCCCCCTCCCGGGACGAGCTGACCAAGAACCA
GGTGTCCCTGACCTGTCTGGTGAAGGGCTTCTACCCCAGCGACATCGCCGTGG
AGTGGGAGAGCAACGGCCAGCCCGAGAACAACTACAAGACCACCCCCCCTGTG
CTGGACAGCGACGGCAGCTTCTTCCTGTACAGCAAGCTGACCGTGGACAAGAG
CCGGTGGCAGCAGGGCAACGTGTTCAGCTGCAGCGTGATGCACGAGGCCCTG
CACAACCACTACACCCAGAAGAGCCTGAGCCTGTCCCCCGGCAAGTGA
SEQ ID 56:
MGWSCIILFLVATATGVHSQVQLVQSGAEVKKPGASVKVSCKASGYAFTDYYMNW
VRQAPGQGLEWMGNINPNNGGTNYNQKFKDRVTMTTDTSTSTAYMELRSLRSDD
TAVYYCARWILYYGRSKWYFDVWGRGTLVTVSSASTKGPSVFPLAPSSKSTSGGT
AALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELAGAPSVFLFPPKPKDTL
MISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQ
QGNVFSCSVMHEALHNHYTQKSLSLSPGK.
SEQ ID 57:
ATGGGATGGTCCTGTATCATCCTGTTTCTGGTGGCCACAGCAACTGGCGTGCAC
TCTCAGGTCCAGCTGGTGCAGAGCGGCGCAGAGGTGAAGAAGCCCGGAGCTA
GCGTCAAGGTCTCCTGCAAGGCTTCAGGCTACGCCTTCACCGACTACTACATGA
ACTGGGTGAGACAGGCTCCAGGACAGGGCCTCGAGTGGATGGGCAACATCAAC
CCCAACAATGGCGGGACAAACTACAACCAGAAGTTCAAGGATCGCGTGACCAT
GACCACCGACACTAGCACCTCAACAGCCTACATGGAGCTGAGGTCTCTGCGGA
GCGATGACACTGCCGTGTACTACTGTGCCAGGTGGATTCTGTACTACGGGAGG
AGCAAGTGGTACTTCGACGTCTGGGGAAGAGGGACACTAGTGACCGTGAGCAG
CGCCAGCACCAAGGGCCCCAGCGTGTTCCCCCTGGCCCCCAGCAGCAAGAGC
ACCAGCGGCGGCACAGCCGCCCTGGGCTGCCTGGTGAAGGACTACTTCCCCG
AGCCCGTGACCGTGTCCTGGAACAGCGGAGCCCTGACAAGCGGGGTGCACAC
CTTCCCCGCCGTGCTGCAGAGCAGCGGCCTGTACAGCCTGAGCAGCGTGGTG
ACAGTGCCCAGCAGCAGCCTGGGCACCCAGACCTACATCTGCAACGTGAACCA
CAAGCCCAGCAACACCAAGGTGGACAAGAAGGTGGAGCCCAAGAGCTGCGAC
AAGACCCACACCTGCCCCCCCTGCCCTGCCCCTGAACTGGCCGGAGCCCCCTC
CGTGTTCCTGTTCCCCCCCAAGCCCAAGGACACCCTGATGATCAGCCGGACCC
CCGAGGTGACCTGCGTGGTGGTGGACGTGAGCCACGAGGACCCTGAGGTGAA
GTTCAATTGGTACGTGGACGGCGTGGAGGTGCACAACGCCAAGACCAAGCCCC
GGGAGGAACAGTACAACAGCACCTACCGGGTGGTGTCCGTGCTGACCGTGCTG
CACCAGGACTGGCTGAACGGCAAAGAATACAAGTGCAAGGTGTCCAACAAGGC
CCTGCCTGCCCCCATCGAGAAAACCATCAGCAAGGCCAAGGGCCAGCCCAGG
GAACCCCAGGTGTACACCCTGCCCCCCTCCCGGGACGAGCTGACCAAGAACCA
GGTGTCCCTGACCTGTCTGGTGAAGGGCTTCTACCCCAGCGACATCGCCGTGG
AGTGGGAGAGCAACGGCCAGCCCGAGAACAACTACAAGACCACCCCCCCTGTG
CTGGACAGCGACGGCAGCTTCTTCCTGTACAGCAAGCTGACCGTGGACAAGAG
CCGGTGGCAGCAGGGCAACGTGTTCAGCTGCAGCGTGATGCACGAGGCCCTG
CACAACCACTACACCCAGAAGAGCCTGAGCCTGTCCCCCGGCAAGTGA
SEQ ID 58:
ATGGGATGGTCCTGCATCATCCTGTTCCTGGTGGCAACTGCCACTGGAGTCCAC
TCCGACATCGTCATGACCCAGAGCCCACTGTCACTCCCCGTGACACCCGGAGA
GCCCGCTAGCATCAGCTGTAGAAGCTCCCAGAGCATCGTGCAGTCTAACGGCG
ATACCTACCTCGAGTGGTACCTGCAGAAGCCCGGACAGTCTCCTCAGCTCCTGA
TTTACCGCGTCAGCAATCGCTTTTCCGGGGTGCCTGATCGGTTTAGCGGCTCAG
GAAGCGGAACCGACTTCACCCTGAAGATCTCAAGGGTGGAGGCTGAGGATGTG
GGCGTGTACTACTGCTTCCAGGGATCTCACGTGCCTTACACCTTCGGACAGGG
CACAAAGCTCGAGATTAAGCGTACGGTGGCCGCTCCCAGCGTGTTCATCTTCCC
CCCCAGCGACGAGCAGCTGAAGAGCGGCACCGCCAGCGTGGTGTGCCTGCTG
AACAACTTCTACCCCCGGGAGGCCAAGGTGCAGTGGAAGGTGGACAACGCCCT
GCAGAGCGGCAACAGCCAGGAAAGCGTCACCGAGCAGGACAGCAAGGACTCC
ACCTACAGCCTGAGCAGCACCCTGACACTGAGCAAGGCCGACTACGAGAAGCA
CAAGGTGTACGCCTGCGAGGTGACCCACCAGGGCCTGTCCAGCCCCGTGACC
AAGAGCTTCAACCGGGGCGAGTGCTAG
SEQ ID 59:
QVQLVQSGAEVKKPGASVKVSCKASGYTFTXaaXaaXaaXaaXaaWVRQAPGQGLE
WMGXaaXaaXaaXaaXaaXaaXaaXaaXaaXaaXaaXaaXaaXaaXaaXaaXaaRVTMTTD
TSTSTAYMELRSLRSDDTAVYYCARXaaXaaXaaXaaXaaXaaXaaXaaXaaXaaXaaXa
aXaaXaaWGRGTLVTVSS
SEQ ID 60:
DIVMTQSPLSLPVTPGEPASISCXaaXaaXaaXaaXaaXaaXaaXaaXaaXaaXaaXaaXa
aXaaXaaXaaWYLQKPGQSPQLLIYXaaXaaXaaXaaXaaXaaXaaGVPDRFSGSGSGT
DFTLKISRVEAEDVGVYYCXaaXaaXaaXaaXaaXaaXaaXaaXaaFGQGTKLEIKRT
SEQ ID 61:
CAGGTGCAGCTGGTGCAGAGCGGAGCCGAGGTGAAGAAGCCTGGCGCCAGCG
TCAAGGTGTCCTGCAAGGCCAGCGGCTACACCTTCACCGACTACTACATGAACT
GGGTGCGGCAGGCCCCAGGCCAGGGACTGGAATGGATGGGCAACATCAACCC
CAACAACGGCGGCACCAACTACAACCAGAAGTTCAAGGACCGGGTCACCATGA
CCACCGACACCAGCACCAGCACCGCCTACATGGAACTGCGGAGCCTGAGAAGC
GACGACACCGCCGTGTACTACTGCGCCCGGTGGATCCTGTACTACGGCCGGTC
CAAGTGGTACTTCGACGTGTGGGGCAGGGGCACACTAGT
SEQ ID NO 62:
GACATCGTGATGACCCAGAGCCCCCTGAGCCTGCCCGTGACCCCTGGCGAGC
CCGCCAGCATCAGCTGCAGAAGCAGCCAGAGCATCGTCCAGAGCAACGGCGA
CACCTACCTGGAATGGTATCTGCAGAAGCCCGGCCAGTCCCCCCAGCTGCTGA
TCTACAGAGTGAGCAACCGGTTCAGCGGCGTGCCCGACAGATTCAGCGGCAGC
GGCTCCGGCACCGACTTCACCCTGAAGATCAGCCGGGTGGAGGCCGAGGACG
TGGGCGTGTACTACTGCTTTCAAGGCAGCCACGTGCCCTACACCTTCGGCCAG
GGCACCAAGCTGGAAATCAAGCGTACG
SEQ ID NO: 63
ACTAGTCACCGTGAGCAGCGCCAGCACCAAGGGCCCCAGCGTGTTCCCCCTGG
CCCCCAGCAGCAAGAGCACCAGCGGCGGCACAGCCGCCCTGGGCTGCCTGGT
GAAGGACTACTTCCCCGAGCCCGTGACCGTGAGCTGGAACAGCGGAGCCCTGA
CCTCCGGCGTGCACACCTTCCCCGCCGTGCTGCAGAGCAGCGGCCTGTACAG
CCTGAGCAGCGTGGTGACCGTGCCCAGCAGCAGCCTGGGCACCCAGACCTAC
ATCTGCAACGTGAACCACAAGCCCAGCAACACCAAGGTGGACAAGAAGGTGGA
GCCCAAGAGCTGCGACAAGACCCACACCTGCCCCCCCTGCCCTGCCCCTGAGC
TGCTGGGCGGACCCGACGTGTTCCTGTTCCCCCCCAAGCCCAAGGACACCCTG
ATGATCAGCCGGACCCCCGAGGTGACCTGCGTGGTGGTGGACGTGAGCCACG
AGGACCCTGAGGTGAAGTTCAATTGGTACGTGGACGGCGTGGAGGTGCACAAC
GCCAAGACCAAGCCCCGGGAGGAACAGTACAACAGCACCTACCGGGTGGTGTC
CGTGCTGACCGTGCTGCACCAGGACTGGCTGAACGGCAAAGAATACAAGTGCA
AGGTGTCCAACAAGGCCCTGCCTGCCCCCGAGGAAAAGACCATCAGCAAGGCC
AAGGGCCAGCCCAGGGAACCCCAGGTGTACACCCTGCCCCCCTCCCGGGACG
AGCTGACCAAGAACCAGGTGTCCCTGACCTGTCTGGTGAAGGGCTTCTACCCC
AGCGACATCGCCGTGGAGTGGGAGAGCAACGGCCAGCCCGAGAACAACTACA
AGACCACCCCCCCTGTGCTGGACAGCGACGGCAGCTTCTTCCTGTACAGCAAG
CTGACCGTGGACAAGAGCCGGTGGCAGCAGGGCAACGTGTTCAGCTGCAGCG
TGATGCACGAGGCCCTGCACAACCACTACACCCAGAAGAGCCTGAGCCTGTCC
CCCGGCAAGTGA
SEQ ID NO: 64
LVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKT
HTCPPCPAPELLGGPDVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPEEK
TISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLSP
GK
SEQ ID NO: 65
ACTAGTCACCGTGAGCAGCGCCAGCACCAAGGGCCCCAGCGTGTTCCCCCTGG
CCCCCAGCAGCAAGAGCACCAGCGGCGGCACAGCCGCCCTGGGCTGCCTGGT
GAAGGACTACTTCCCCGAGCCCGTGACCGTGAGCTGGAACAGCGGAGCCCTGA
CCTCCGGCGTGCACACCTTCCCCGCCGTGCTGCAGAGCAGCGGCCTGTACAG
CCTGAGCAGCGTGGTGACCGTGCCCAGCAGCAGCCTGGGCACCCAGACCTAC
ATCTGCAACGTGAACCACAAGCCCAGCAACACCAAGGTGGACAAGAAGGTGGA
GCCCAAGAGCTGCGACAAGACCCACACCTGCCCCCCCTGCCCTGCCCCTGAGC
TGCTGGGCGGACCCGACGTGTTCCTGTTCCCCCCCAAGCCCAAGGACACCCTG
ATGATCAGCCGGACCCCCGAGGTGACCTGCGTGGTGGTGGACGTGAGCCACG
AGGACCCTGAGGTGAAGTTCAATTGGTACGTGGACGGCGTGGAGGTGCACAAC
GCCAAGACCAAGCCCCGGGAGGAACAGTACAACAGCACCTACCGGGTGGTGTC
CGTGCTGACCGTGCTGCACCAGGACTGGCTGAACGGCAAAGAATACAAGTGCA
AGGTGTCCAACAAGGCCCTGCCTCTGCCCGAGGAAAAGACCATCAGCAAGGCC
AAGGGCCAGCCCAGGGAACCCCAGGTGTACACCCTGCCCCCCTCCCGGGACG
AGCTGACCAAGAACCAGGTGTCCCTGACCTGTCTGGTGAAGGGCTTCTACCCC
AGCGACATCGCCGTGGAGTGGGAGAGCAACGGCCAGCCCGAGAACAACTACA
AGACCACCCCCCCTGTGCTGGACAGCGACGGCAGCTTCTTCCTGTACAGCAAG
CTGACCGTGGACAAGAGCCGGTGGCAGCAGGGCAACGTGTTCAGCTGCAGCG
TGATGCACGAGGCCCTGCACAACCACTACACCCAGAAGAGCCTGAGCCTGTCC
CCCGGCAAGTGA
SEQ ID NO: 66
LVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKT
HTCPPCPAPELLGGPDVFLFPPKPKDTLMISRTPEVTCVVVDVSHEDPEVKFNWYV
DGVEVHNAKTKPREEQYNSTYRVVSVLTVLHQDWLNGKEYKCKVSNKALPLPEEK
TISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLSP
GK
SEQ ID NO: 67
ATGGGCTGGTCCTGCATCATCCTGTTTCTGGTGGCCACCGCCACCGGCGTGCA
CAGCCAGGTGCAGCTGGTGCAGAGCGGAGCCGAGGTGAAGAAGCCTGGCGCC
AGCGTCAAGGTGTCCTGCAAGGCCAGCGGCTACACCTTCACCGACTACTACAT
GAACTGGGTGCGGCAGGCCCCAGGCCAGGGACTGGAATGGATGGGCAACATC
AACCCCAACAACGGCGGCACCAACTACAACCAGAAGTTCAAGGACCGGGTCAC
CATGACCACCGACACCAGCACCAGCACCGCCTACATGGAACTGCGGAGCCTGA
GAAGCGACGACACCGCCGTGTACTACTGCGCCCGGTGGATCCTGTACTACGGC
CGGTCCAAGTGGTACTTCGACGTGTGGGGCAGGGGCACACTAGTCACCGTGAG
CAGCGCCAGCACCAAGGGCCCCAGCGTGTTCCCCCTGGCCCCCAGCAGCAAG
AGCACCAGCGGCGGCACAGCCGCCCTGGGCTGCCTGGTGAAGGACTACTTCC
CCGAGCCCGTGACCGTGAGCTGGAACAGCGGAGCCCTGACCTCCGGCGTGCA
CACCTTCCCCGCCGTGCTGCAGAGCAGCGGCCTGTACAGCCTGAGCAGCGTG
GTGACCGTGCCCAGCAGCAGCCTGGGCACCCAGACCTACATCTGCAACGTGAA
CCACAAGCCCAGCAACACCAAGGTGGACAAGAAGGTGGAGCCCAAGAGCTGC
GACAAGACCCACACCTGCCCCCCCTGCCCTGCCCCTGAGCTGCTGGGCGGAC
CCGACGTGTTCCTGTTCCCCCCCAAGCCCAAGGACACCCTGATGATCAGCCGG
ACCCCCGAGGTGACCTGCGTGGTGGTGGACGTGAGCCACGAGGACCCTGAGG
TGAAGTTCAATTGGTACGTGGACGGCGTGGAGGTGCACAACGCCAAGACCAAG
CCCCGGGAGGAACAGTACAACAGCACCTACCGGGTGGTGTCCGTGCTGACCGT
GCTGCACCAGGACTGGCTGAACGGCAAAGAATACAAGTGCAAGGTGTCCAACA
AGGCCCTGCCTGCCCCCGAGGAAAAGACCATCAGCAAGGCCAAGGGCCAGCC
CAGGGAACCCCAGGTGTACACCCTGCCCCCCTCCCGGGACGAGCTGACCAAG
AACCAGGTGTCCCTGACCTGTCTGGTGAAGGGCTTCTACCCCAGCGACATCGC
CGTGGAGTGGGAGAGCAACGGCCAGCCCGAGAACAACTACAAGACCACCCCC
CCTGTGCTGGACAGCGACGGCAGCTTCTTCCTGTACAGCAAGCTGACCGTGGA
CAAGAGCCGGTGGCAGCAGGGCAACGTGTTCAGCTGCAGCGTGATGCACGAG
GCCCTGCACAACCACTACACCCAGAAGAGCCTGAGCCTGTCCCCCGGCAAGTG
A
SEQ ID NO: 68
MGWSCIILFLVATATGVHSQVQLVQSGAEVKKPGASVKVSCKASGYTFTDYYMNW
VRQAPGQGLEWMGNINPNNGGTNYNQKFKDRVTMTTDTSTSTAYMELRSLRSDD
TAVYYCARWILYYGRSKWYFDVWGRGTLVTVSSASTKGPSVFPLAPSSKSTSGGT
AALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPDVFLFPPKPKDTL
MISRTPEVTCVVVDVSHEDPEVKFNWYVDGVEVHNAKTKPREEQYNSTYRVVSVL
TVLHQDWLNGKEYKCKVSNKALPAPEEKTISKAKGQPREPQVYTLPPSRDELTKNQ
VSLTCLVKGFYPSDIAVEWESNGQPENNYKTTPPVLDSDGSFFLYSKLTVDKSRWQ
QGNVFSCSVMHEALHNHYTQKSLSLSPGK
SEQ ID NO: 69
ATGGGCTGGTCCTGCATCATCCTGTTTCTGGTGGCCACCGCCACCGGCGTGCA
CAGCGACATCGTGATGACCCAGAGCCCCCTGAGCCTGCCCGTGACCCCTGGC
GAGCCCGCCAGCATCAGCTGCAGAAGCAGCCAGAGCATCGTCCAGAGCAACG
GCGACACCTACCTGGAATGGTATCTGCAGAAGCCCGGCCAGTCCCCCCAGCTG
CTGATCTACAGAGTGAGCAACCGGTTCAGCGGCGTGCCCGACAGATTCAGCGG
CAGCGGCTCCGGCACCGACTTCACCCTGAAGATCAGCCGGGTGGAGGCCGAG
GACGTGGGCGTGTACTACTGCTTTCAAGGCAGCCACGTGCCCTACACCTTCGG
CCAGGGCACCAAGCTGGAAATCAAGCGTACGGTGGCCGCCCCCAGCGTGTTCA
TCTTCCCCCCCAGCGATGAGCAGCTGAAGAGCGGCACCGCCAGCGTGGTGTGT
CTGCTGAACAACTTCTACCCCCGGGAGGCCAAGGTGCAGTGGAAGGTGGACAA
TGCCCTGCAGAGCGGCAACAGCCAGGAGAGCGTGACCGAGCAGGACAGCAAG
GACTCCACCTACAGCCTGAGCAGCACCCTGACCCTGAGCAAGGCCGACTACGA
GAAGCACAAGGTGTACGCCTGTGAGGTGACCCACCAGGGCCTGTCCAGCCCC
GTGACCAAGAGCTTCAACCGGGGCGAGTGCTGA
Seq ID NO: 70
ATGGGCTGGTCCTGCATCATCCTGTTTCTGGTGGCCACCGCCACCGGCGTGCA
CAGCCAGGTGCAGCTGGTGCAGAGCGGAGCCGAGGTGAAGAAGCCTGGCGCC
AGCGTCAAGGTGTCCTGCAAGGCCAGCGGCTACACCTTCACCGACTACTACAT
GAACTGGGTGCGGCAGGCCCCAGGCCAGGGACTGGAATGGATGGGCAACATC
AACCCCAACAACGGCGGCACCAACTACAACCAGAAGTTCAAGGACCGGGTCAC
CATGACCACCGACACCAGCACCAGCACCGCCTACATGGAACTGCGGAGCCTGA
GAAGCGACGACACCGCCGTGTACTACTGCGCCCGGTGGATCCTGTACTACGGC
CGGTCCAAGTGGTACTTCGACGTGTGGGGCAGGGGCACACTAGTGACCGTGTC
CAGCGCCAGCACCAAGGGCCCCAGCGTGTTCCCCCTGGCCCCCAGCAGCAAG
AGCACCAGCGGCGGCACAGCCGCCCTGGGCTGCCTGGTGAAGGACTACTTCC
CCGAACCGGTGACCGTGTCCTGGAACAGCGGAGCCCTGACCAGCGGCGTGCA
CACCTTCCCCGCCGTGCTGCAGAGCAGCGGCCTGTACAGCCTGAGCAGCGTG
GTGACCGTGCCCAGCAGCAGCCTGGGCACCCAGACCTACATCTGTAACGTGAA
CCACAAGCCCAGCAACACCAAGGTGGACAAGAAGGTGGAGCCCAAGAGCTGTG
ACAAGACCCACACCTGCCCCCCCTGCCCTGCCCCCGAGCTGCTGGGAGGCCC
CAGCGTGTTCCTGTTCCCCCCCAAGCCTAAGGACACCCTGATGATCAGCAGAAC
CCCCGAGGTGACCTGTGTGGTGGTGGATGTGAGCCACGAGGACCCTGAGGTG
AAGTTCAACTGGTACGTGGACGGCGTGGAGGTGCACAATGCCAAGACCAAGCC
CAGGGAGGAGCAGTACAACAGCACCTACCGGGTGGTGTCCGTGCTGACCGTG
CTGCACCAGGATTGGCTGAACGGCAAGGAGTACAAGTGTAAGGTGTCCAACAA
GGCCCTGCCTGCCCCTATCGAGAAAACCATCAGCAAGGCCAAGGGCCAGCCCA
GAGAGCCCCAGGTGTACACCCTGCCCCCTAGCAGAGATGAGCTGACCAAGAAC
CAGGTGTCCCTGACCTGCCTGGTGAAGGGCTTCTACCCCAGCGACATCGCCGT
GGAGTGGGAGAGCAACGGCCAGCCCGAGAACAACTACAAGACCACCCCCCCT
GTGCTGGACAGCGATGGCAGCTTCTTCCTGTACAGCAAGCTGACCGTGGACAA
GAGCAGATGGCAGCAGGGCAACGTGTTCAGCTGCTCCGTGATGCACGAGGCC
CTGCACAATCACTACACCCAGAAGAGCCTGAGCCTGTCCCCTGGCAAGTGATG
A
SEQ ID NO: 71
CAGGTGCAGCTGCAGCAGCCTGGAGCCGAGCTGGTGAAGCCCGGCGCCAGCG
TGAAAATGTCCTGCAAGGCCAGCGGCTACACCTTCACCAGCTACAACATGCACT
GGGTGAAGCAGACCCCCGGCAGGGGCCTCGAGTGGATCGGAGCTATCTACCC
CGGCAACGGCGACACTAGCTACAACCAGAAGTTCAAGGGCAAGGCCACCCTGA
CCGCCGACAAGAGCAGCAGCACCGCCTACATGCAGCTGAGCAGCCTGACCAG
CGAGGACAGCGCCGTGTATTACTGCGCCAGGAGCACCTACTACGGCGGCGACT
GGTACTTCAACGTCTGGGGCGCCGGCACACTAGTGACCGTGTCCAGCGCCAGC
ACCAAGGGCCCCAGCGTGTTCCCCCTGGCCCCCAGCAGCAAGAGCACCAGCG
GCGGCACAGCCGCCCTGGGCTGCCTGGTGAAGGACTACTTCCCCGAACCGGT
GACCGTGTCCTGGAACAGCGGAGCCCTGACCAGCGGCGTGCACACCTTCCCC
GCCGTGCTGCAGAGCAGCGGCCTGTACAGCCTGAGCAGCGTGGTGACCGTGC
CCAGCAGCAGCCTGGGCACCCAGACCTACATCTGTAACGTGAACCACAAGCCC
AGCAACACCAAGGTGGACAAGAAGGTGGAGCCCAAGAGCTGTGACAAGACCCA
CACCTGCCCCCCCTGCCCTGCCCCCGAGCTGCTGGGAGGCCCCAGCGTGTTC
CTGTTCCCCCCCAAGCCTAAGGACACCCTGATGATCAGCAGAACCCCCGAGGT
GACCTGTGTGGTGGTGGATGTGAGCCACGAGGACCCTGAGGTGAAGTTCAACT
GGTACGTGGACGGCGTGGAGGTGCACAATGCCAAGACCAAGCCCAGGGAGGA
GCAGTACAACAGCACCTACCGGGTGGTGTCCGTGCTGACCGTGCTGCACCAGG
ATTGGCTGAACGGCAAGGAGTACAAGTGTAAGGTGTCCAACAAGGCCCTGCCT
GCCCCTATCGAGAAAACCATCAGCAAGGCCAAGGGCCAGCCCAGAGAGCCCCA
GGTGTACACCCTGCCCCCTAGCAGAGATGAGCTGACCAAGAACCAGGTGTCCC
TGACCTGCCTGGTGAAGGGCTTCTACCCCAGCGACATCGCCGTGGAGTGGGAG
AGCAACGGCCAGCCCGAGAACAACTACAAGACCACCCCCCCTGTGCTGGACAG
CGATGGCAGCTTCTTCCTGTACAGCAAGCTGACCGTGGACAAGAGCAGATGGC
AGCAGGGCAACGTGTTCAGCTGCTCCGTGATGCACGAGGCCCTGCACAATCAC
TACACCCAGAAGAGCCTGAGCCTGTCCCCTGGCAAGTGA
SEQ ID NO: 72
CAGGTACAACTGCAGCAGCCTGGGGCTGAGCTGGTGAAGCCTGGGGCCTCAG
TGAAGATGTCCTGCAAGGCTTCTGGCTACACATTTACCAGTTACAATATGCACTG
GGTAAAACAGACACCTGGTCGGGGCCTGGAATGGATTGGAGCTATTTATCCCG
GAAATGGTGATACTTCCTACAATCAGAAGTTCAAAGGCAAGGCCACATTGACTG
CAGACAAATCCTCCAGCACAGCCTACATGCAGCTCAGCAGCCTGACATCTGAG
GACTCTGCGGTCTATTACTGTGCAAGATCGACTTACTACGGCGGTGACTGGTAC
TTCAATGTCTGGGGCGCAGGGACACTAGTCACCGTGAGCAGCGCCAGCACCAA
GGGCCCCAGCGTGTTCCCCCTGGCCCCCAGCAGCAAGAGCACCAGCGGCGGC
ACAGCCGCCCTGGGCTGCCTGGTGAAGGACTACTTCCCCGAGCCCGTGACCGT
GAGCTGGAACAGCGGAGCCCTGACCTCCGGCGTGCACACCTTCCCCGCCGTG
CTGCAGAGCAGCGGCCTGTACAGCCTGAGCAGCGTGGTGACCGTGCCCAGCA
GCAGCCTGGGCACCCAGACCTACATCTGCAACGTGAACCACAAGCCCAGCAAC
ACCAAGGTGGACAAGAAGGTGGAGCCCAAGAGCTGCGACAAGACCCACACCTG
CCCCCCCTGCCCTGCCCCTGAGCTGCTGGGCGGACCCGACGTGTTCCTGTTCC
CCCCCAAGCCCAAGGACACCCTGATGATCAGCCGGACCCCCGAGGTGACCTGC
GTGGTGGTGGACGTGAGCCACGAGGACCCTGAGGTGAAGTTCAATTGGTACGT
GGACGGCGTGGAGGTGCACAACGCCAAGACCAAGCCCCGGGAGGAACAGTAC
AACAGCACCTACCGGGTGGTGTCCGTGCTGACCGTGCTGCACCAGGACTGGCT
GAACGGCAAAGAATACAAGTGCAAGGTGTCCAACAAGGCCCTGCCTGCCCCCG
AGGAAAAGACCATCAGCAAGGCCAAGGGCCAGCCCAGGGAACCCCAGGTGTA
CACCCTGCCCCCCTCCCGGGACGAGCTGACCAAGAACCAGGTGTCCCTGACCT
GTCTGGTGAAGGGCTTCTACCCCAGCGACATCGCCGTGGAGTGGGAGAGCAAC
GGCCAGCCCGAGAACAACTACAAGACCACCCCCCCTGTGCTGGACAGCGACG
GCAGCTTCTTCCTGTACAGCAAGCTGACCGTGGACAAGAGCCGGTGGCAGCAG
GGCAACGTGTTCAGCTGCAGCGTGATGCACGAGGCCCTGCACAACCACTACAC
CCAGAAGAGCCTGAGCCTGTCCCCCGGCAAGTGA
SEQ ID NO: 73
CAGATCGTCCTGAGCCAGAGCCCCGCCATTCTGAGCGCCAGCCCCGGCGAGA
AAGTGACCATGACCTGCAGGGCCTCCAGCAGCGTGAGCTACATCCACTGGTTC
CAGCAGAAGCCCGGCAGCTCACCCAAGCCCTGGATCTACGCCACCAGCAACCT
CGCCTCTGGCGTGCCCGTGAGGTTCAGCGGAAGCGGCAGCGGCACCAGCTAC
TCCCTGACCATCAGCAGGGTGGAGGCAGAGGACGCCGCCACCTACTACTGCCA
GCAGTGGACCAGCAACCCCCCAACCTTCGGCGGCGGCACAAAGCTGGAGATCA
AGCGTACGGTGGCCGCCCCCAGCGTGTTCATCTTCCCCCCCAGCGATGAGCAG
CTGAAGAGCGGCACCGCCAGCGTGGTGTGTCTGCTGAACAACTTCTACCCCCG
GGAGGCCAAGGTGCAGTGGAAGGTGGACAATGCCCTGCAGAGCGGCAACAGC
CAGGAGAGCGTGACCGAGCAGGACAGCAAGGACTCCACCTACAGCCTGAGCA
GCACCCTGACCCTGAGCAAGGCCGACTACGAGAAGCACAAGGTGTACGCCTGT
GAGGTGACCCACCAGGGCCTGTCCAGCCCCGTGACCAAGAGCTTCAACCGGG
GCGAGTGCTGA
SEQ ID NO: 74
QVQLQQPGAELVKPGASVKMSCKASGYTFTSYNMHWVKQTPGRGLEWIGAIYPG
NGDTSYNQKFKGKATLTADKSSSTAYMQLSSLTSEDSAVYYCARSTYYGGDWYFN
VWGAGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKS
LSLSPGK
SEQ ID NO: 75
QVQLQQPGAELVKPGASVKMSCKASGYTFTSYNMHWVKQTPGRGLEWIGAIYPG
NGDTSYNQKFKGKATLTADKSSSTAYMQLSSLTSEDSAVYYCARSTYYGGDWYFN
VWGAGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKVEP
KSCDKTHTCPPCPAPELLGGPDVFLFPPKPKDTLMISRTPEVTCVVVDVSHEDPEV
KFNWYVDGVEVHNAKTKPREEQYNSTYRVVSVLTVLHQDWLNGKEYKCKVSNKAL
PAPEEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQK
SLSLSPGK.
SEQ ID NO: 76
QIVLSQSPAILSASPGEKVTMTCRASSSVSYIHWFQQKPGSSPKPWIYATSNLASGV
PVRFSGSGSGTSYSLTISRVEAEDAATYYCQQWTSNPPTFGGGTKLEIKRTVAAPS
VFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC
SEQ ID: 77
MPLLLLLPLLWAGALAGMRTEDLPKAVVFLEPQWYRVLEKDSVTLKCQGAYSPEDN
STQWFHNESLISSQASSYFIDAATVDDSGEYRCQTNLSTLSDPVQLEVHIGWLLLQA
PRWVFKEEDPIHLRCHSWKNTALHKVTYLQNGKGRKYFHHNSDFYIPKATLKDSGS
YFCRGLVGSKNVSSETVNITITQGLAVSTISSFFPPGYQHHHHHHHHHH.
SEQ ID: 78
MPLLLLLPLLWAGALAGMRTEDLPKAVVFLEPQWYRVLEKDSVTLKCQGAYSPEDN
STQWFHNESLISSQASSYFIDAATVDDSGEYRCQTNLSTLSDPVQLEVHIGWLLLQA
PRWVFKEEDPIHLRCHSWKNTALHKVTYLQNGKGRKYFHHNSDFYIPKATLKDSGS
YFCRGLFGSKNVSSETVNITITQGLAVSTISSFFPPGYQHHHHHHHHHH.

We claim:

1. An antibody preparation comprising antibodies which comprise an immunoglobulin heavy chain constant region, or antigen binding fragments thereof which are linked to an immunoglobulin heavy chain constant region wherein said immunoglobulin heavy chain constant region confers an effector function to the antibody or antigen binding fragment, and wherein said antibody or antigen binding fragment specifically binds to a growth factor receptor and wherein said immunoglobulin heavy chain constant region is mutated in at least 2 positions and has an altered glycosylation profile such that the ratio of fucose to mannose is 0.8:3 or less so that said antibody or antigen binding fragment has an enhanced effector function in comparison with an equivalent antibody or antigen-binding fragment with an immunoglobulin heavy chain constant region lacking said mutations and altered glycosylation profile.

2. The antibody preparation of claim 1, wherein the growth factor receptor is selected from IGF-1R, EGFR, HER-2 or HER-3.

3. The antibody preparation of claim 2 wherein the growth factor receptor is human IGF-1R.

4. The antibody preparation of claim 1, wherein the heavy chain constant region is derived from the IgG isotype.

5. The antibody preparation of claim 4, wherein the heavy chain constant region is derived from IgG1.

6. The antibody preparation of claim 4, wherein the heavy chain constant region comprises at least one CH2 domain from IgG3 and at least one constant heavy chain domain from IgG1.

7. The antibody preparation of claim 5, wherein at least one of the CH2 domains is from IgGI and wherein said mutations are in positions 239 and 332 of IgG1.

8. The antibody preparation of claim 7, wherein the mutations are S239D and I332E.

9. The antibody preparation of claim 7, wherein the heavy chain constant region of the antibody or antigen binding fragment thereof has a further mutation in position 330.

10. The antibody preparation of claim 9, wherein the 330 mutation is A330L.

11. The antibody preparation of claim 1, wherein the heavy chain constant region of the antibody or antigen binding fragment thereof, comprises an N-glycoside linked sugar chain, which has reduced fucose levels when compared to the levels of fucose found in the equivalent wild type heavy chain constant region.

12. The antibody preparation of claim 11, wherein the ratio of fucose to mannose in the total N-glycoside linked sugar chain is at least 0.5:3.

13. The antibody preparation of claim 11, wherein the N-glycoside linked sugar chain does not contain bound fucose.

14. The antibody preparation according to claim 1, wherein the antibody or antigen binding fragment thereof is humanised or chimaeric.

15. The antibody preparation according to claim 1, wherein the antibody or antigen binding fragment thereof additionally binds primate IGF-1R.

16. The antibody preparation according to claim 1, wherein the antibody is monoclonal.

17. A method of producing the antibody preparation according to claim 1, comprising expressing in a cell line an antibody or antigen binding fragment thereof which has been adapted to regulate the presence or absence of binding of fucose to an N-glycoside linked sugar chain which binds to the immunologically functional molecule.

18. The method of claim 16, wherein the cell line is a mammalian cell line.

19. The method of claim 17, wherein the cell line is a CHO cell line.

20. A method according to claim 17, wherein said antibody or antigen binding fragment thereof is secreted by said host cell into a culture media.

21. A method according to claim 20, wherein said antibody or antigen binding fragment thereof is further purified to at least 95% or greater with respect to said antibody or antigen binding fragment containing serum-free culture media.

22. A pharmaceutical composition comprising an antibody preparation according to claim 1, and a pharmaceutically acceptable carrier.

23. A kit-of-parts comprising the composition according to claim 22 together with instructions for use.

24. A method of treating a human patient afflicted with cancer which method comprises the step of administering a therapeutically effective amount of the antibody preparation of claim 1.

25. A method according to claim 24, wherein the patient is afflicted with breast cancer.

26. A method according to claim 24, wherein the patient is afflicted with prostate cancer.

27. Use of an antibody preparation according to claim 1 in the manufacture of a medicament for the treatment of a disease or disorder selected from the group consisting of; rheumatoid arthritis, breast cancer, prostate cancer, lung cancer or myeloma.

28. An antibody preparation according to claim 1, wherein the antibody or antigen binding fragment thereof neutralises the activity of IGF-1R.


SEQUENCE LISTING (70 sequences)

SEQ ID NO: 1 - protein, 14 aa, Mus Musculus
WILYYGRSKWYFDV

SEQ ID NO: 2 - protein, 17 aa, Mus Musculus
NINPNNGGTNYNQKFKD

SEQ ID NO: 3 - protein, 5 aa, Mus Musculus
DYYMN

SEQ ID NO: 4 - protein, 16 aa, Mus Musculus
RSSQSIVQSNGDTYLE

SEQ ID NO: 5 - protein, 7 aa, Mus Musculus
RISNRFS

SEQ ID NO: 6 - protein, 9 aa, Mus Musculus
FQGSHVPYT

SEQ ID NO: 7 - protein, 7 aa, Mus Musculus
RVSNRFS

SEQ ID NO: 8 - protein, 123 aa, Mus Musculus
EVQLQQSGPELVKPGASVRISCKASGYAFTDYYMNWVKQSHGKSLEWVANINPNNGGTNYNQKFKDKATLTVDKSSNTAYMELRSLTSEDTAVYYCARWILYYGRSKWYFDVWGTGTTVTVSS

SEQ ID NO: 9 - protein, 114 aa, Mus Musculus
DVLMTQTPLSLPVSLGDHASISCRSSQSIVQSNGDTYLEWYLQKPGQSPKLLIYRISNRFSGVPDRFSGSGSGTDFTLKISRVEAEDLGVYYCFQGSHVPYTFGGGTKLEIKRA

SEQ ID NO: 10 - protein, 122 aa, Mus Musculus
QVQLKQSGPGLVQSSQSLSITCTISGFSLTSHGIYWLRQSPGKGLEWLGVIWSGGSADYNAAFISRLSISKDNSKSQVFFKMNSLQADDTAIYYCARSPYYYRSSLYAMDYWGQGTSVTVSS

SEQ ID NO: 11 - protein, 109 aa, Mus Musculus
NIVLTQSPKSMSMSIGERVTLSCKASENVGTYVSWYQQKAEQSPKLLIYGASNRHTGVPDRFTGSGSSTDFTLTISSVQAEDLADYHCGQSYSDPLTFGAGTKLELKRA

SEQ ID NO: 12 - protein, 123 aa, Artificial Sequence
  Chimeric variable heavy chain comprising sequences from mus musculus and homo sapiens
EVQLQQSGPELVKPGASVRISCKASGYAFTDYYMNWVKQSHGKSLEWVANINPNNGGTNYNQKFKDKATLTVDKSSNTAYMELRSLTSEDTAVYYCARWILYYGRSKWYFDVWGTGTLVTVSS

SEQ ID NO: 13 - protein, 114 aa, Artificial Sequence
  Chimeric variable heavy chain comprising sequences from mus musculus and homo sapiens
DVLMTQTPLSLPVSLGDHASISCRSSQSIVQSNGDTYLEWYLQKPGQSPKLLIYRISNRFSGVPDRFSGSGSGTDFTLKISRVEAEDLGVYYCFQGSHVPYTFGGGTKLEIKRT

SEQ ID NO: 14 - protein, 123 aa, Homo sapiens
QVQLVQSGAEVKKPGASVKVSCKASGYTFTDYYMNWVRQAPGQGLEWMGNINPNNGGTNYNQKFKDRVTMTTDTSTSTAYMELRSLRSDDTAVYYCARWILYYGRSKWYFDVWGRGTLVTVSS

SEQ ID NO: 15 - protein, 123 aa, Homo Sapiens
QVQLVQSGAEVKKPGASVKVSCKASGYAFTDYYMNWVRQAPGQGLEWMGNINPNNGGTNYNQKFKDRVTMTTDTSTSTAYMELRSLRSDDTAVYYCARWILYYGRSKWYFDVWGRGTLVTVSS

SEQ ID NO: 16 - protein, 114 aa, Homo Sapiens
DIVMTQSPLSLPVTPGEPASISCRSSQSIVQSNGDTYLEWYLQKPGQSPQLLIYRVSNRFSGVPDRFSGSGSGTDFTLKISRVEAEDVGVYYCFQGSHVPYTFGQGTKLEIKRT

SEQ ID NO: 17 - protein, 15 aa, Artificial Sequence
  Biotinylated tag sequence
GLNDIFEAQKIEWHE

SEQ ID NO: 18 - protein, 123 aa, Mus Musculus
EVQLQQSGPELVKPGASVRISCKASGYAFTDYYMNWVKQSHGKSLEWMANINPNNGGTNYNQKFKDKATLTVDKSSNTAYMELRSLTSEDSAVYYCARWILYYGRSKWYFDVWGPGTTVTVSS

SEQ ID NO: 19 - protein, 114 aa, Mus Musculus
DVLMTQSPLSLPVSLGDHASISCRSSQSIVQSNGDTYLEWYLQKPGQSPKLLIYRVSNRFSGVPDRFSGSGSGTDFTLKISRVEAEDLGVYYCFQGSHVPYTFGGGTKLEIKRA

SEQ ID NO: 20 - protein, 123 aa, Mus Musculus
EVQLQQSGPELVKPGASVKISCKASGYAFTDYYMNWVKQTHGRSLEWMANINPNTGGTNYNQKFRGKATLTVDKSSTTAYMELRSLTSEDSAVYYCARWILYYGSSRWYFDVWGTGTTVTVSS

SEQ ID NO: 21 - protein, 114 aa, Mus Musculus
DVLMTQTPLSLPVSLGDQASISCRSSQTIVHSNGNTYLEWYLQKPGQSPKLLIYKVSNRFSGVPDRFSGSGSGTDFTLKISRVEAEDLGIYYCFQGSHVPYTFGGGTKLEIKRA

SEQ ID NO: 22 - protein, 123 aa, Mus Musculus
EVQLQQSGPELVKPGASVKISCKASGYAFTDYYMNWVKQSHGKSLEWMANINPNTGGTNYNQKFTGKATLTVDKSSTTAYMELRSLTSEDSAVYYCTRWILYYGSSKWYFDVWGTGTTVTVSS

SEQ ID NO: 23 - protein, 114 aa, Mus Musculus
DVLMTQTPLSLPVSLGDQASISCRSSQTIVHSNGNTYLEWYLQKPGQSPKLLIYRVSYRFSGVPDRFSGSGSGTDFTLKISRLEAEDLGIYYCFQGSHVPYTFGGGTKLEIKRA

SEQ ID NO: 24 - protein, 472 aa, Artificial Sequence
  Chimeric variable heavy chain comprising sequences from mus musculus and homo sapiens
MGWSWIFFFLLSETAGVLSEVQLQQSGPELVKPGASVRISCKASGYAFTDYYMNWVKQSHGKSLEWVANINPNNGGTNYNQKFKDKATLTVDKSSNTAYMELRSLTSEDTAVYYCARWILYYGRSKWYFDVWGTGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 238 aa, Artificial Sequence
  Chimeric variable heavy chain comprising sequences from mus musculus and homo sapiens
MKLPVRLVVLMFWIPASSSDVLMTQTPLSLPVSLGDHASISCRSSQSIVQSNGDTYLEWYLQKPGQSPKLLIYRISNRFSGVPDRFSGSGSGTDFTLKISRVEAEDLGVYYCFQGSHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 369 bp, Mus Musculus
gaggtccagctgcaacaatctggacctgagctggtgaagcctggggcttcagtgaggatatcctgtaaggcttctggatacgcgttcactgactactacatgaactgggtgaagcagagccatggaaagagccttgagtgggtggcaaatattaatcccaacaatggtggtactaactacaaccagaagttcaaggacaaggccacattgactgtagacaagtcctccaacacagcctacatggagctccgcagtctgacatctgaggacactgcagtctattactgtgcaagatggattctttactacggtcgtagcaaatggtacttcgatgtctggggcacagggaccacggtcaccgtctcctcg

SEQ ID NO: 27 - DNA, 342 bp, Mus Musculus
gatgttttgatgacccaaactccactctccctgcctgtcagtcttggagatcacgcctccatctcttgcagatctagtcagagtattgttcaaagtaatggagacacctatttagaatggtacctgcagaaaccaggccagtctccaaagctcctgatctacagaatttccaaccgattttctggggtcccagacaggttcagtggcagtggatcagggacagatttcacactcaagatcagtagagtggaggctgaggatctgggagtttattactgctttcagggttcacatgttccgtacacgttcggaggggggaccaagctggaaataaaacgggct

SEQ ID NO: 28 - DNA, 369 bp, Mus Musculus
gaggtccagctgcaacaatctggacctgagctggtgaagcctggggcttcagtgaggatatcctgtaaggcttctggatacgcgttcactgactactacatgaactgggtgaaacagagccatggaaagagccttgagtggatggcaaatattaatcccaacaatggtggtactaactacaaccagaagttcaaggacaaggccacattgactgtagacaagtcctccaacacagcctacatggagctccgcagtctgacatctgaggactctgcagtctattactgtgcaagatggattctttactacggtcgtagcaagtggtacttcgatgtctggggcccagggaccacggtcaccgtctcctcg

SEQ ID NO: 29 - DNA, 342 bp, Mus Musculus
gatgttttgatgacccaaagtccactctccctgcctgtcagtcttggagatcacgcctccatctcttgcagatctagtcagagcattgttcaaagtaatggagacacctatttagaatggtacctgcagaaaccaggccagtctccaaagctcctgatctatagagtttccaaccgattttctggggtcccagacaggttcagtggcagtggatcagggacagatttcacactcaagatcagtagagtggaggctgaggatctgggagtttattactgctttcagggttcacatgttccgtacacgttcggaggggggaccaagctggaaataaaacgggct

SEQ ID NO: 30 - DNA, 369 bp, Artificial Sequence
  Chimeric variable heavy chain comprising sequences from mus musculus and homo sapiens
gaggtccagctgcaacaatctggacctgagctggtgaagcctggggcttcagtgaggatatcctgtaaggcttctggatacgcgttcactgactactacatgaactgggtgaagcagagccatggaaagagccttgagtgggtggcaaatattaatcccaacaatggtggtactaactacaaccagaagttcaaggacaaggccacattgactgtagacaagtcctccaacacagcctacatggagctccgcagtctgacatctgaggacactgcagtctattactgtgcaagatggattctttactacggtcgtagcaaatggtacttcgatgtctggggcacagggacactagtcacagtctcctca

SEQ ID NO: 31 - DNA, 342 bp, Artificial Sequence
  Chimeric variable heavy chain comprising sequences from mus musculus and homo sapiens
gatgttttgatgacccaaactccactctccctgcctgtcagtcttggagatcacgcctccatctcttgcagatctagtcagagtattgttcaaagtaatggagacacctatttagaatggtacctgcagaaaccaggccagtctccaaagctcctgatctacagaatttccaaccgattttctggggtcccagacaggttcagtggcagtggatcagggacagatttcacactcaagatcagtagagtggaggctgaggatctgggagtttattactgctttcagggttcacatgttccgtacacgttcggaggggggaccaagctggaaataaaacgtacg

SEQ ID NO: 32 - DNA, 1419 bp, Artificial Sequence
  Chimeric variable heavy chain comprising sequences from mus musculus and homo sapiens
atgggatggagctggatctttttcttcctcctgtcagaaactgcaggtgtcctctctgaggtccagctgcaacaatctggacctgagctggtgaagcctggggcttcagtgaggatatcctgtaaggcttctggatacgcgttcactgactactacatgaactgggtgaagcagagccatggaaagagccttgagtgggtggcaaatattaatcccaacaatggtggtactaactacaaccagaagttcaaggacaaggccacattgactgtagacaagtcctccaacacagcctacatggagctccgcagtctgacatctgaggacactgcagtctattactgtgcaagatggattctttactacggtcgtagcaaatggtacttcgatgtctggggcacagggacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 33 - DNA, 717 bp, Artificial Sequence
  Chimeric variable heavy chain comprising sequences from mus musculus and homo sapiens
atgaagttgcctgttcggctcgtggtgctgatgttctggattcctgcttccagcagtgatgttttgatgacccaaactccactctccctgcctgtcagtcttggagatcacgcctccatctcttgcagatctagtcagagtattgttcaaagtaatggagacacctatttagaatggtacctgcagaaaccaggccagtctccaaagctcctgatctacagaatttccaaccgattttctggggtcccagacaggttcagtggcagtggatcagggacagatttcacactcaagatcagtagagtggaggctgaggatctgggagtttattactgctttcagggttcacatgttccgtacacgttcggaggggggaccaagctggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggacaacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 34 - DNA, 369 bp, Homo sapiens
caggtccagctggtgcagagcggcgcagaggtgaagaagcccggagctagcgtcaaggtctcctgcaaggcttcaggctacacattcaccgactactacatgaactgggtgagacaggctccaggacagggcctcgagtggatgggcaacatcaaccccaacaatggcgggacaaactacaaccagaagttcaaggatcgcgtgaccatgaccaccgacactagcacctcaacagcctacatggagctgaggtctctgcggagcgatgacactgccgtgtactactgtgccaggtggattctgtactacgggaggagcaagtggtacttcgacgtctggggaagagggacactagtgaccgtgagcagc

SEQ ID NO: 35 - DNA, 369 bp, Homo sapiens
caggtccagctggtgcagagcggcgcagaggtgaagaagcccggagctagcgtcaaggtctcctgcaaggcttcaggctacgccttcaccgactactacatgaactgggtgagacaggctccaggacagggcctcgagtggatgggcaacatcaaccccaacaatggcgggacaaactacaaccagaagttcaaggatcgcgtgaccatgaccaccgacactagcacctcaacagcctacatggagctgaggtctctgcggagcgatgacactgccgtgtactactgtgccaggtggattctgtactacgggaggagcaagtggtacttcgacgtctggggaagagggacactagtgaccgtgagcagc

SEQ ID NO: 36 - DNA, 342 bp, Homo sapiens
gacatcgtcatgacccagagcccactgtcactccccgtgacacccggagagcccgctagcatcagctgtagaagctcccagagcatcgtgcagtctaacggcgatacctacctcgagtggtacctgcagaagcccggacagtctcctcagctcctgatttaccgcgtcagcaatcgcttttccggggtgcctgatcggtttagcggctcaggaagcggaaccgacttcaccctgaagatctcaagggtggaggctgaggatgtgggcgtgtactactgcttccagggatctcacgtgccttacaccttcggacagggcacaaagctcgagattaagcgtacg

SEQ ID NO: 37 - protein, 472 aa, Homo sapiens
MGWSCIILFLVATATGVHSQVQLVQSGAEVKKPGASVKVSCKASGYTFTDYYMNWVRQAPGQGLEWMGNINPNNGGTNYNQKFKDRVTMTTDTSTSTAYMELRSLRSDDTAVYYCARWILYYGRSKWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 472 aa, Homo sapiens
MGWSCIILFLVATATGVHSQVQLVQSGAEVKKPGASVKVSCKASGYAFTDYYMNWVRQAPGQGLEWMGNINPNNGGTNYNQKFKDRVTMTTDTSTSTAYMELRSLRSDDTAVYYCARWILYYGRSKWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 238 aa, Homo sapiens
MGWSCIILFLVATATGVHSDIVMTQSPLSLPVTPGEPASISCRSSQSIVQSNGDTYLEWYLQKPGQSPQLLIYRVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 1419 bp, Homo sapiens
atgggatggtcctgtatcatcctgtttctggtggccacagcaactggcgtgcactctcaggtccagctggtgcagagcggcgcagaggtgaagaagcccggagctagcgtcaaggtctcctgcaaggcttcaggctacacattcaccgactactacatgaactgggtgagacaggctccaggacagggcctcgagtggatgggcaacatcaaccccaacaatggcgggacaaactacaaccagaagttcaaggatcgcgtgaccatgaccaccgacactagcacctcaacagcctacatggagctgaggtctctgcggagcgatgacactgccgtgtactactgtgccaggtggattctgtactacgggaggagcaagtggtacttcgacgtctggggaagagggacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaagtga

SEQ ID NO: 41 - DNA, 1419 bp, Homo sapiens
atgggatggtcctgtatcatcctgtttctggtggccacagcaactggcgtgcactctcaggtccagctggtgcagagcggcgcagaggtgaagaagcccggagctagcgtcaaggtctcctgcaaggcttcaggctacgccttcaccgactactacatgaactgggtgagacaggctccaggacagggcctcgagtggatgggcaacatcaaccccaacaatggcgggacaaactacaaccagaagttcaaggatcgcgtgaccatgaccaccgacactagcacctcaacagcctacatggagctgaggtctctgcggagcgatgacactgccgtgtactactgtgccaggtggattctgtactacgggaggagcaagtggtacttcgacgtctggggaagagggacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaagtga

SEQ ID NO: 42 - DNA, 717 bp, Homo sapiens
atgggatggtcctgcatcatcctgttcctggtggcaactgccactggagtccactccgacatcgtcatgacccagagcccactgtcactccccgtgacacccggagagcccgctagcatcagctgtagaagctcccagagcatcgtgcagtctaacggcgatacctacctcgagtggtacctgcagaagcccggacagtctcctcagctcctgatttaccgcgtcagcaatcgcttttccggggtgcctgatcggtttagcggctcaggaagcggaaccgacttcaccctgaagatctcaagggtggaggctgaggatgtgggcgtgtactactgcttccagggatctcacgtgccttacaccttcggacagggcacaaagctcgagattaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgctga

SEQ ID NO: 43 - protein, 19 aa, Artificial Sequence
  Campath leader sequence
MGWSCIILFLVATATGVHS

SEQ ID NO: 44 - protein, 1367 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 45 - protein, 1367 aa, cynomolgus macaque
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSACGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTWKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDLEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKDEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 46 - protein, 1373 aa, Mus Musculus
MKSGSGGGSPTSLWGLVFLSAALSLWPTSGEICGPGIDIRNDYQQLKRLENCTVIEGFLHILLISKAEDYRSYRFPKLTVITEYLLLFRVAGLESLGDLFPNLTVIRGWKLFYNYALVIFEMTNLKDIGLYNLRNITRGAIRIEKNADLCYLSTIDWSLILDAVSNNYIVGNKPPKECGDLCPGTLEEKPMCEKTTINNEYNYRCWTTNRCQKMCPSVCGKRACTENNECCHPECLGSCHTPDDNTTCVACRHYYYKGVCVPACPPGTYRFEGWRCVDRDFCANIPNAESSDSDGFVIHDDECMQECPSGFIRNSTQSMYCIPCEGPCPKVCGDEEKKTKTIDSVTSAQMLQGCTILKGNLLINIRRGNNIASELENFMGLIEVVTGYVKIRHSHALVSLSFLKNLRLILGEEQLEGNYSFYVLDNQNLQQLWDWNHRNLTVRSGKMYFAFNPKLCVSEIYRMEEVTGTKGRQSKGDINTRNNGERASCESDVLRFTSTTTWKNRIIITWHRYRPPDYRDLISFTVYYKEAPFKNVTEYDGQDACGSNSWNMVDVDLPPNKEGEPGILLHGLKPWTQYAVYVKAVTLTMVENDHIRGAKSEILYIRTNASVPSIPLDVLSASNSSSQLIVKWNPPTLPNGNLSYYIVRWQRQPQDGYLYRHNYCSKDKIPIRKYADGTIDVEEVTENPKTEVCGGDKGPCCACPKTEAEKQAEKEEAEYRKVFENFLHNSIFVPRPERRRRDVMQVANTTMSSRSRNTTVADTYNITDPEEFETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVPAKTTYENFMHLIIALPVAILLIVGGLVIMLYVFHRKRNNSRLGNGVLYASVNPEYFSAADVYVPDEWEVAREKITMNRELGQGSFGMVYEGVAKGVVKDEPETRVAIKTVNEAASMRERIEFLNEASVMKEFNCHHVVRLLGVVSQGQPTLVIMELMTRGDLKSYLRSLRPEVEQNNLVLIPPSLSKMIQMAGEIADGMAYLNANKFVHRDLAARNCMVAEDFTVKIGDFGMTRDIYETDYYRKGGKGLLPVRWMSPESLKDGVFTTHSDVWSFGVVLWEIATLAEQPYQGLSNEQVLRFVMEGGLLDKPDNCPDMLFELMRMCWQYNPKMRPSFLEIIGSIKDEMEPSFQEVSFYYSEENKPPEPEELEMELEMEPENMESVPLDPSASSASLPLPERHSGHKAENGPGPGVLVLRASFDERQPYAHMNGGRANERALPLPQSSTC

SEQ ID NO: 47 - protein, 1180 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHAAAIEGRSGSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLRRASLG

SEQ ID NO: 48 - protein, 1180 aa, Cynomolgus macaque
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSACGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTWKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDLEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHAAAIEGRSGSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLRRASLG

SEQ ID NO: 49 - protein, 71 aa, Homo sapiens
MGPETLCGAELVDALQFVCGDRGFYFNKPTGYGSSSRRAPQTGIVDECCFRSCDLRRLEMYCAPLKPAKSA

SEQ ID NO: 50 - protein, 86 aa, Homo sapiens
MGPETLCGAELVDALQFVCGDRGFYFNKPTGYGSSSRRAPQTGIVDECCFRSCDLRRLEMYCAPLKPAKSAGLNDIFEAQKIEWHE

SEQ ID NO: 51 - protein, 68 aa, Homo sapiens
MAYRPSETLCGGELVDTLQFVCGDRGFYFSRPASRVSRRSRGIVEECCFRSCDLALLETYCATPAKSE

SEQ ID NO: 52 - protein, 83 aa, Homo sapiens
MAYRPSETLCGGELVDTLQFVCGDRGFYFSRPASRVSRRSRGIVEECCFRSCDLALLETYCATPAKSEGLNDIFEAQKIEWHE

SEQ ID NO: 53 - protein, 1382 aa, Homo sapiens
MGTGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKIIIGPLIFVFLFSVVIGSIYLFLRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPNMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPS

SEQ ID NO: 54 - protein, 472 aa, Homo sapiens
MGWSCIILFLVATATGVHSQVQLVQSGAEVKKPGASVKVSCKASGYTFTDYYMNWVRQAPGQGLEWMGNINPNNGGTNYNQKFKDRVTMTTDTSTSTAYMELRSLRSDDTAVYYCARWILYYGRSKWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - DNA, 1419 bp, Homo sapiens
atgggatggtcctgtatcatcctgtttctggtggccacagcaactggcgtgcactctcaggtccagctggtgcagagcggcgcagaggtgaagaagcccggagctagcgtcaaggtctcctgcaaggcttcaggctacacattcaccgactactacatgaactgggtgagacaggctccaggacagggcctcgagtggatgggcaacatcaaccccaacaatggcgggacaaactacaaccagaagttcaaggatcgcgtgaccatgaccaccgacactagcacctcaacagcctacatggagctgaggtctctgcggagcgatgacactgccgtgtactactgtgccaggtggattctgtactacgggaggagcaagtggtacttcgacgtctggggaagagggacactagtgaccgtgagcagcgccagcaccaagggccccagcgtgttccccctggcccccagcagcaagagcaccagcggcggcacagccgccctgggctgcctggtgaaggactacttccccgagcccgtgaccgtgtcctggaacagcggagccctgacaagcggggtgcacaccttccccgccgtgctgcagagcagcggcctgtacagcctgagcagcgtggtgacagtgcccagcagcagcctgggcacccagacctacatctgcaacgtgaaccacaagcccagcaacaccaaggtggacaagaaggtggagcccaagagctgcgacaagacccacacctgccccccctgccctgcccctgaactggccggagccccctccgtgttcctgttcccccccaagcccaaggacaccctgatgatcagccggacccccgaggtgacctgcgtggtggtggacgtgagccacgaggaccctgaggtgaagttcaattggtacgtggacggcgtggaggtgcacaacgccaagaccaagccccgggaggaacagtacaacagcacctaccgggtggtgtccgtgctgaccgtgctgcaccaggactggctgaacggcaaagaatacaagtgcaaggtgtccaacaaggccctgcctgcccccatcgagaaaaccatcagcaaggccaagggccagcccagggaaccccaggtgtacaccctgcccccctcccgggacgagctgaccaagaaccaggtgtccctgacctgtctggtgaagggcttctaccccagcgacatcgccgtggagtgggagagcaacggccagcccgagaacaactacaagaccaccccccctgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgtcccccggcaagtga

SEQ ID NO: 56 - protein, 472 aa, Homo sapiens
MGWSCIILFLVATATGVHSQVQLVQSGAEVKKPGASVKVSCKASGYAFTDYYMNWVRQAPGQGLEWMGNINPNNGGTNYNQKFKDRVTMTTDTSTSTAYMELRSLRSDDTAVYYCARWILYYGRSKWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - DNA, 1419 bp, Homo sapiens
atgggatggtcctgtatcatcctgtttctggtggccacagcaactggcgtgcactctcaggtccagctggtgcagagcggcgcagaggtgaagaagcccggagctagcgtcaaggtctcctgcaaggcttcaggctacgccttcaccgactactacatgaactgggtgagacaggctccaggacagggcctcgagtggatgggcaacatcaaccccaacaatggcgggacaaactacaaccagaagttcaaggatcgcgtgaccatgaccaccgacactagcacctcaacagcctacatggagctgaggtctctgcggagcgatgacactgccgtgtactactgtgccaggtggattctgtactacgggaggagcaagtggtacttcgacgtctggggaagagggacactagtgaccgtgagcagcgccagcaccaagggccccagcgtgttccccctggcccccagcagcaagagcaccagcggcggcacagccgccctgggctgcctggtgaaggactacttccccgagcccgtgaccgtgtcctggaacagcggagccctgacaagcggggtgcacaccttccccgccgtgctgcagagcagcggcctgtacagcctgagcagcgtggtgacagtgcccagcagcagcctgggcacccagacctacatctgcaacgtgaaccacaagcccagcaacaccaaggtggacaagaaggtggagcccaagagctgcgacaagacccacacctgccccccctgccctgcccctgaactggccggagccccctccgtgttcctgttcccccccaagcccaaggacaccctgatgatcagccggacccccgaggtgacctgcgtggtggtggacgtgagccacgaggaccctgaggtgaagttcaattggtacgtggacggcgtggaggtgcacaacgccaagaccaagccccgggaggaacagtacaacagcacctaccgggtggtgtccgtgctgaccgtgctgcaccaggactggctgaacggcaaagaatacaagtgcaaggtgtccaacaaggccctgcctgcccccatcgagaaaaccatcagcaaggccaagggccagcccagggaaccccaggtgtacaccctgcccccctcccgggacgagctgaccaagaaccaggtgtccctgacctgtctggtgaagggcttctaccccagcgacatcgccgtggagtgggagagcaacggccagcccgagaacaactacaagaccaccccccctgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgtcccccggcaagtga

SEQ ID NO: 58 - DNA, 717 bp, Homo sapiens
atgggatggtcctgcatcatcctgttcctggtggcaactgccactggagtccactccgacatcgtcatgacccagagcccactgtcactccccgtgacacccggagagcccgctagcatcagctgtagaagctcccagagcatcgtgcagtctaacggcgatacctacctcgagtggtacctgcagaagcccggacagtctcctcagctcctgatttaccgcgtcagcaatcgcttttccggggtgcctgatcggtttagcggctcaggaagcggaaccgacttcaccctgaagatctcaagggtggaggctgaggatgtgggcgtgtactactgcttccagggatctcacgtgccttacaccttcggacagggcacaaagctcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgacactgagcaaggccgactacgagaagcacaaggtgtacgcctgcgaggtgacccaccagggcctgtccagccccgtgaccaagagcttcaaccggggcgagtgctag

SEQ ID NO: 59 - protein, 123 aa, Homo sapiens
  VARIANT 31, 32, 33, 34, 35, 50, 51, 52, 53, 54, 55, 56, 57, 58,59, 60, 61, 62, 63, 64, 65, 66, 99, 100, 101, 102, 103, 104,105, 106, 107, 108, 109, 110, 111, 112: Xaa = Any Amino Acid
QVQLVQSGAEVKKPGASVKVSCKASGYTFTXXXXXWVRQAPGQGLEWMGXXXXXXXXXXXXXXXXXRVTMTTDTSTSTAYMELRSLRSDDTAVYYCARXXXXXXXXXXXXXXWGRGTLVTVSS

SEQ ID NO: 60 - protein, 114 aa, Homo sapiens
  VARIANT 24, 25, 26, 27, 28, 29, 30, 31, 32, 33, 34, 35, 36, 37,38, 39, 55, 56, 57, 58, 59, 60, 61, 94, 95, 96, 97, 98, 99,100, 101, 102: Xaa = Any Amino Acid
  VARIANT 24, 25, 26, 27, 28, 29, 30, 31, 32, 33, 34, 35, 36, 37,38, 39, 55, 56, 57, 58, 59, 60, 61, 94, 95, 96, 97, 98, 99,100, 101, 102: Xaa = Any Amino Acid
DIVMTQSPLSLPVTPGEPASISCXXXXXXXXXXXXXXXXWYLQKPGQSPQLLIYXXXXXXXGVPDRFSGSGSGTDFTLKISRVEAEDVGVYYCXXXXXXXXXFGQGTKLEIKRT

SEQ ID NO: 61 - DNA, 356 bp, Homo sapiens
caggtgcagctggtgcagagcggagccgaggtgaagaagcctggcgccagcgtcaaggtgtcctgcaaggccagcggctacaccttcaccgactactacatgaactgggtgcggcaggccccaggccagggactggaatggatgggcaacatcaaccccaacaacggcggcaccaactacaaccagaagttcaaggaccgggtcaccatgaccaccgacaccagcaccagcaccgcctacatggaactgcggagcctgagaagcgacgacaccgccgtgtactactgcgcccggtggatcctgtactacggccggtccaagtggtacttcgacgtgtggggcaggggcacactagt

SEQ ID NO: 62 - DNA, 342 bp, Homo sapiens
gacatcgtgatgacccagagccccctgagcctgcccgtgacccctggcgagcccgccagcatcagctgcagaagcagccagagcatcgtccagagcaacggcgacacctacctggaatggtatctgcagaagcccggccagtccccccagctgctgatctacagagtgagcaaccggttcagcggcgtgcccgacagattcagcggcagcggctccggcaccgacttcaccctgaagatcagccgggtggaggccgaggacgtgggcgtgtactactgctttcaaggcagccacgtgccctacaccttcggccagggcaccaagctggaaatcaagcgtacg

SEQ ID NO: 63 - DNA, 1012 bp, Homo sapiens
actagtcaccgtgagcagcgccagcaccaagggccccagcgtgttccccctggcccccagcagcaagagcaccagcggcggcacagccgccctgggctgcctggtgaaggactacttccccgagcccgtgaccgtgagctggaacagcggagccctgacctccggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccctgcccctgagctgctgggcggacccgacgtgttcctgttcccccccaagcccaaggacaccctgatgatcagccggacccccgaggtgacctgcgtggtggtggacgtgagccacgaggaccctgaggtgaagttcaattggtacgtggacggcgtggaggtgcacaacgccaagaccaagccccgggaggaacagtacaacagcacctaccgggtggtgtccgtgctgaccgtgctgcaccaggactggctgaacggcaaagaatacaagtgcaaggtgtccaacaaggccctgcctgcccccgaggaaaagaccatcagcaaggccaagggccagcccagggaaccccaggtgtacaccctgcccccctcccgggacgagctgaccaagaaccaggtgtccctgacctgtctggtgaagggcttctaccccagcgacatcgccgtggagtgggagagcaacggccagcccgagaacaactacaagaccaccccccctgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgtcccccggcaagtga

SEQ ID NO: 64 - protein, 336 aa, Homo sapiens
LVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 1012 bp, Homo sapiens
actagtcaccgtgagcagcgccagcaccaagggccccagcgtgttccccctggcccccagcagcaagagcaccagcggcggcacagccgccctgggctgcctggtgaaggactacttccccgagcccgtgaccgtgagctggaacagcggagccctgacctccggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccctgcccctgagctgctgggcggacccgacgtgttcctgttcccccccaagcccaaggacaccctgatgatcagccggacccccgaggtgacctgcgtggtggtggacgtgagccacgaggaccctgaggtgaagttcaattggtacgtggacggcgtggaggtgcacaacgccaagaccaagccccgggaggaacagtacaacagcacctaccgggtggtgtccgtgctgaccgtgctgcaccaggactggctgaacggcaaagaatacaagtgcaaggtgtccaacaaggccctgcctctgcccgaggaaaagaccatcagcaaggccaagggccagcccagggaaccccaggtgtacaccctgcccccctcccgggacgagctgaccaagaaccaggtgtccctgacctgtctggtgaagggcttctaccccagcgacatcgccgtggagtgggagagcaacggccagcccgagaacaactacaagaccaccccccctgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgtcccccggcaagtga

SEQ ID NO: 66 - protein, 336 aa, Homo sapiens
LVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - DNA, 1419 bp, Homo sapiens
atgggctggtcctgcatcatcctgtttctggtggccaccgccaccggcgtgcacagccaggtgcagctggtgcagagcggagccgaggtgaagaagcctggcgccagcgtcaaggtgtcctgcaaggccagcggctacaccttcaccgactactacatgaactgggtgcggcaggccccaggccagggactggaatggatgggcaacatcaaccccaacaacggcggcaccaactacaaccagaagttcaaggaccgggtcaccatgaccaccgacaccagcaccagcaccgcctacatggaactgcggagcctgagaagcgacgacaccgccgtgtactactgcgcccggtggatcctgtactacggccggtccaagtggtacttcgacgtgtggggcaggggcacactagtcaccgtgagcagcgccagcaccaagggccccagcgtgttccccctggcccccagcagcaagagcaccagcggcggcacagccgccctgggctgcctggtgaaggactacttccccgagcccgtgaccgtgagctggaacagcggagccctgacctccggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccctgcccctgagctgctgggcggacccgacgtgttcctgttcccccccaagcccaaggacaccctgatgatcagccggacccccgaggtgacctgcgtggtggtggacgtgagccacgaggaccctgaggtgaagttcaattggtacgtggacggcgtggaggtgcacaacgccaagaccaagccccgggaggaacagtacaacagcacctaccgggtggtgtccgtgctgaccgtgctgcaccaggactggctgaacggcaaagaatacaagtgcaaggtgtccaacaaggccctgcctgcccccgaggaaaagaccatcagcaaggccaagggccagcccagggaaccccaggtgtacaccctgcccccctcccgggacgagctgaccaagaaccaggtgtccctgacctgtctggtgaagggcttctaccccagcgacatcgccgtggagtgggagagcaacggccagcccgagaacaactacaagaccaccccccctgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgtcccccggcaagtga

SEQ ID NO: 68 - protein, 472 aa, Homo sapiens
MGWSCIILFLVATATGVHSQVQLVQSGAEVKKPGASVKVSCKASGYTFTDYYMNWVRQAPGQGLEWMGNINPNNGGTNYNQKFKDRVTMTTDTSTSTAYMELRSLRSDDTAVYYCARWILYYGRSKWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - DNA, 717 bp, Homo sapiens
atgggctggtcctgcatcatcctgtttctggtggccaccgccaccggcgtgcacagcgacatcgtgatgacccagagccccctgagcctgcccgtgacccctggcgagcccgccagcatcagctgcagaagcagccagagcatcgtccagagcaacggcgacacctacctggaatggtatctgcagaagcccggccagtccccccagctgctgatctacagagtgagcaaccggttcagcggcgtgcccgacagattcagcggcagcggctccggcaccgacttcaccctgaagatcagccgggtggaggccgaggacgtgggcgtgtactactgctttcaaggcagccacgtgccctacaccttcggccagggcaccaagctggaaatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgctga

SEQ ID NO: 70 - DNA, 1422 bp, Homo sapiens
atgggctggtcctgcatcatcctgtttctggtggccaccgccaccggcgtgcacagccaggtgcagctggtgcagagcggagccgaggtgaagaagcctggcgccagcgtcaaggtgtcctgcaaggccagcggctacaccttcaccgactactacatgaactgggtgcggcaggccccaggccagggactggaatggatgggcaacatcaaccccaacaacggcggcaccaactacaaccagaagttcaaggaccgggtcaccatgaccaccgacaccagcaccagcaccgcctacatggaactgcggagcctgagaagcgacgacaccgccgtgtactactgcgcccggtggatcctgtactacggccggtccaagtggtacttcgacgtgtggggcaggggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaagtgatga